(19) United States
(12) Patent Application Publication
(10) Pub. No.: US 2019/0071502 A1
(43) Pub. Date: Mar. 7, 2019

(54) METHODS AND COMPOSITIONS FOR TARGETING A COMPLEX COMPRISING NON-CLASSICAL HLA-I AND NEOANTIGEN IN CANCER

(71) Applicant: AbeXXa Biologics, Inc., Arlington, TX (US)

(72) Inventor: Jon WEIDANZ, Arlington, TX (US)

(21) Appl. No.: 16/103,764

(22) Filed: Aug. 14, 2018

Related U.S. Application Data
(63) Continuation of application No. PCT/US2018/015086, filed on Jan. 24, 2018.
(60) Provisional application No. 62/460,585, filed on Feb. 17, 2017.
(60) Provisional application No. 62/449,954, filed on Jan. 24, 2017.

(51) Int. Cl. C07K 16/28; A61P 35/00; C07K 16/30

(52) U.S. Cl. CPC C07K 16/2833; A61P 35/00; C07K 2317/31; C07K 16/2815; C07K 16/30

(57) ABSTRACT

Disclosed herein are methods and compositions for targeting a complex comprising a non-classical HLA-I and a neoantigen in cancer. Further disclosed herein are antibodies that selectively bind to a complex comprising a non-classical HLA-I and a neoantigen, as well as methods of use thereof.

### CROSS-REFERENCE

This application is a continuation of International Application No. PCT/US2018/015086, filed on Jan. 24, 2018, which claims the benefit of U.S. Provisional Application No. 62/449,954 filed Jan. 24, 2017; and U.S. Provisional Application No. 62/460,585 filed Feb. 17, 2017, which applications are incorporated by reference herein in their entirety.

### SEQUENCE LISTING

The instant application contains a Sequence Listing which has been submitted electronically in ASCII format and is hereby incorporated by reference in its entirety. Said ASCII copy, created on Jan. 23, 2018, is named 50626701301_SL.txt and is 15,190 bytes in size.

### SUMMARY OF THE DISCLOSURE

Disclosed herein, in some embodiments, are methods and compositions for targeting a complex comprising a non-classical HLA-I and a neoantigen in cancer. In some embodiments, methods and compositions comprise antibodies that selectively bind to a complex comprising a non-classical HLA-I and a neoantigen, thereby modulating an immune response against cancer cells.

Disclosed herein, in certain embodiments, are antibodies that selectively bind to a complex comprising a non-classical HLA-I and a peptide. In some instances, the antibody does not have a binding affinity to (i) the non-classical HLA-I alone; or (ii) the peptide alone. In some instances, the peptide is expressed by an antigen processing machinery (APM)-proficient cell. In some instances, the peptide is expressed by a TAP1/2-proficient cell. In some instances, the peptide is expressed by an antigen processing machinery (APM)-deficient cell. In some instances, the peptide is expressed by a TAP1/2-deficient cell.

In some instances, the peptide comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), SEQ ID NO: 31 (VMAPRTLVL), SEQ ID NO: 19 (YLLPRRGPRL), SEQ ID NO: 20 (AISPRTLNA), SEQ ID NO: 21 (SQAPLPCVL), SEQ ID NO: 15 (YLLEMLWRL), SEQ ID NO: 16 (YMLDLQPETT), SEQ ID NO: 22 (QMRPVSRVL), SEQ ID NO: 23 (ALALVRMLI), SEQ ID NO: 24 (SQQPYLQLQ), SEQ ID NO: 25 (AMAPIKTHL), SEQ ID NO: 26 (AMAPIKVRL), SEQ ID NO: 17 (YLLPAIVHI), SEQ ID NO: 27 (ILDQKINEV), SEQ ID NO: 28 (GVYDGEEHSV), SEQ ID NO: 29 (KVLEYVIKV), SEQ ID NO: 18 (SLLMWITQV), SEQ ID NO: 30 (YLEPGPVTV), SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the peptide comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), or SEQ ID NO: 31 (VMAPRTLVL). In some instances, the peptide comprises, consisting essentially of, or consisting of a sequence according to SEQ ID NO. 3 (VMAPRTLFL).

In some instances, the peptide comprises, consisting essentially of, or consisting of a sequence according to SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI). In some instances, the peptide comprises, consisting essentially of, or consisting of a sequence according to SEQ ID NO: 35 (MLALLTQVA), SEQ ID NO: 1 (GLADKVYFL), or SEQ ID NO: 2 (ILSPTVVSI). In some instances, the peptide comprises, consisting essentially of, or consisting of a sequence according to SEQ ID NO. 35 (MLALLTQVA). In some instances, the peptide comprises, consisting essentially of, or consisting of a sequence according to SEQ ID NO. 1 (GLADKVYFL). In some instances, the peptide comprises, consisting essentially of, or consisting of a sequence according to SEQ ID NO. 2 (ILSPTVVSI).

In some instances, the non-classical HLA-I is HLA- E, HLA-F, HLA-G, or HLA-H. In some instances, the non-classical HLA-I is HLA-E. In some instances, the HLA-E is HLA-E*0101 or HLA-E*0103. In some instances, the antibody selectively binds to the complex comprising the HLA-E and the peptide. In some instances, the antibody selectively binds to the complex comprising: (a) the HLA-E*0101 and the peptide; (b) the HLA-E*0103 and the peptide; or (c) the HLA-E*0101 and the peptide, and the HLA-E*0103 and the peptide.

In some instances, the complex comprises the HLA-E and VMAPRTLFL (SEQ ID NO: 3), the HLA-E and VMAPRTLIL (SEQ ID NO: 13), the HLA-E and VMPPRTLLL (SEQ ID NO: 14), the HLA-E and YLLPRRGPRL (SEQ ID NO: 19), the HLA-E and AISPRTLNA (SEQ ID NO: 20), the HLA-E and SQAPLPCVL (SEQ ID NO: 21), the HLA-E and YLLEMLWRL (SEQ ID NO: 15), the HLA-E and YMLDLQPETT (SEQ ID NO: 16), the HLA-E and QMRPVSRVL (SEQ ID NO: 22), the HLA-E and ALALVRMLI (SEQ ID NO: 23), the HLA-E and SQQPYLQLQ (SEQ ID NO: 24), the HLA-E and AMAPIKTHL (SEQ ID NO: 25), the HLA-E and AMAPIKVRL (SEQ ID NO: 26), the HLA-E and YLLPAIVHI (SEQ ID NO: 17), the HLA-E and ILDQKINEV (SEQ ID NO: 27), the HLA-E and GVYDGEEHSV (SEQ ID NO: 28), the HLA-E and KVLEYVIKV (SEQ ID NO: 29), the HLA-E and SLLMWITQV (SEQ ID NO: 18), the HLA-E and YLEPGPVTV (SEQ ID NO: 30), the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO: 3), the HLA-E and VMAPRTLIL (SEQ ID NO: 13), or the HLA-E and VMPPRTLLL (SEQ ID NO: 14). In some instances, the complex comprises the HLA-E and VMAPRTLFL (SEQ ID NO. 3).

In some instances, the complex comprises the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2). In some instances, the complex comprises the HLA-E and MLALLTQVA (SEQ ID NO: 35), the HLA-E and GLADKVYFL (SEQ ID NO: 1), or the HLA-E and ILSPTVVSI (SEQ ID NO: 2). In some instances, the complex comprises the HLA-E and MLALLTQVA (SEQ ID NO. 35). In some instances, the complex comprises the HLA-E and GLADKVYFL (SEQ ID NO. 1). In some instances, the complex comprises the HLA-E and ILSPTVVSI (SEQ ID NO. 2).

In some instances, the antibody is a murine antibody, a chimeric antibody, a camelid antibody, a humanized antibody, or a human antibody. In some instances, the antibody is a T-cell receptor-like (TCR-like) antibody. In some instances, the antibody is a single domain antibody. In some instances, the single domain antibody is a camelid single domain antibody. In some instances, the antibody is a multispecific antibody. In some instances, the antibody is a multifunctional antibody. In some instances, the antibody further comprises a conjugated therapeutic moiety.

In some instances, the selective binding of the antibody to the complex comprising the non-classical HLA-I and the peptide induces an immune response in a cell. In some instances, the immune response comprises activation of T cells. In some instances, the T cell is a CD8+ T cell. In some instances, the immune response comprises activation of cytotoxic T cells (CTL_{S}). In some instances, the cell is a cancer cell.

Disclosed herein, in certain embodiments, are methods of treating cancer in an individual in need thereof, comprising administering to the individual an antibody that selectively binds to a complex comprising a non-classical HLA-I and a neoantigen. In some instances, the antibody does not have a binding affinity to (i) the non-classical HLA-I alone; or (ii) the neoantigen alone. In some instances, the neoantigen is expressed by an antigen processing machinery (APM)-proficient cell. In some instances, the neoantigen is expressed by a TAP1/2-proficient cell. In some instances, the neoantigen is expressed by an antigen processing machinery (APM)-deficient cell. In some instances, the neoantigen is expressed by a TAP1/2-deficient cell.

In some instances, the neoantigen comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), SEQ ID NO: 31 (VMAPRTLVL), SEQ ID NO: 19 (YLLPRRGPRL), SEQ ID NO: 20 (AISPRTLNA), SEQ ID NO: 21 (SQAPLPCVL), SEQ ID NO: 15 (YLLEMLWRL), SEQ ID NO: 16 (YMLDLQPETT), SEQ ID NO: 22 (QMRPVSRVL), SEQ ID NO: 23 (ALALVRMLI), SEQ ID NO: 24 (SQQPYLQLQ), SEQ ID NO: 25 (AMAPIKTHL), SEQ ID NO: 26 (AMAPIKVRL), SEQ ID NO: 17 (YLLPAIVHI), SEQ ID NO: 27 (ILDQKINEV), SEQ ID NO: 28 (GVYDGEEHSV), SEQ ID NO: 29 (KVLEYVIKV), SEQ ID NO: 18 (SLLMWITQV), SEQ ID NO: 30 (YLEPGPVTV), SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the neoantigen comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), or SEQ ID NO: 31 (VMAPRTLVL). In some instances, the neoantigen comprises, consisting essential of, or consisting of a sequence according to SEQ ID NO. 3 (VMAPRTLFL).

In some instances, the neoantigen comprises, consisting essentially of, or consisting of a sequence according to SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI). In some instances, the neoantigen comprises, consisting essentially of, or consisting of a sequence according to SEQ ID NO: 35 (MLALLTQVA), SEQ ID NO: 1 (GLADKVYFL), or SEQ ID NO: 2 (ILSPTVVSI). In some instances, the neoantigen comprises, consisting essentially of, or consisting of a sequence according to SEQ ID NO. 35 (MLALLTQVA). In some instances, the neoantigen comprises, consisting essentially of, or consisting of a sequence according to SEQ ID NO. 1 (GLADKVYFL). In some instances, the neoantigen comprises, consisting essentially of, or consisting of a sequence according to SEQ ID NO. 2 (ILSPTVVSI).

In some instances, the non-classical HLA-I is HLA-E, HLA-F, HLA-G, or HLA-H. In some instances, the non-classical HLA-I is HLA-E. In some instances, the HLA-E is HLA-E*0101 or HLA-E*0103. In some instances, the antibody selectively binds to the complex comprising the HLA-E and the neoantigen. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen.

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), HLA-E and VMAPRTLVL (SEQ ID NO: 31), HLA-E and YLLPRRGPRL (SEQ ID NO: 19), the HLA-E and AISPRTLNA (SEQ ID NO: 20), the HLA-E and SQAPLPCVL (SEQ ID NO: 21), the HLA-E and YLLEMLWRL (SEQ ID NO: 15), the HLA-E and YMLDLQPETT (SEQ ID NO: 16), the HLA-E and QMRPVSRVL (SEQ ID NO: 22), the HLA-E and ALALVRMLI (SEQ ID NO: 23), the HLA-E and SQQPYLQLQ (SEQ ID NO: 24), the HLA-E and AMAPIKTHL (SEQ ID NO: 25), the HLA-E and AMAPIKVRL (SEQ ID NO: 26), the HLA-E and YLLPAIVHI (SEQ ID NO: 17), the HLA-E and ILDQKINEV (SEQ ID NO: 27), the HLA-E and GVYDGEEHSV (SEQ ID NO: 28), the HLA-E and KVLEYVIKV (SEQ ID NO: 29), the HLA-E and SLLMWITQV (SEQ ID NO: 18), the HLA-E and YLEPGPVTV (SEQ ID NO: 30), the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), or HLA-E and VMAPRTLVL (SEQ ID NO: 31). In some instances, the complex comprises the HLA-E and VMAPRTLFL (SEQ ID NO. 3).

In some instances, the complex comprises the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2). In some instances, the complex comprises the HLA-E and MLALLTQVA (SEQ ID NO: 35), the HLA-E and GLADKVYFL (SEQ ID NO: 1), or the HLA-E and ILSPTVVSI (SEQ ID NO: 2). In some instances, the complex comprises the HLA-E and MLALLTQVA (SEQ ID NO. 35). In some instances, the complex comprises the HLA-E and GLADKVYFL (SEQ ID NO. 1). In some instances, the complex comprises the HLA-E and ILSPTVVSI (SEQ ID NO. 2).

In some instances, the antibody is a murine antibody, a chimeric antibody, a camelid antibody, a humanized antibody, or a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody. In some instances, the antibody further comprises a conjugated therapeutic moiety.

In some instances, the selective binding of the antibody to the complex comprising the non-classical HLA-I and the neoantigen induces an immune response. In some instances, the immune response comprises activation of T cells. In some instances, the T cell is a CD8+ T cell. In some instances, the immune response comprises activation of cytotoxic T cells (CTLs).

In some instances, the antibody is administered continuously for 1, 2, 3, 4, 5, 6, 7, 8, 9, 10, 14, 15, 28, 30 or more days. In some instances, the antibody is administered at predetermined time intervals for 1, 2, 3, 4, 5, 6, 7, 8, 9, 10, 14, 15, 28, 30 or more days. In some instances, the antibody is administered is administered intermittently for 1, 2, 3, 4, 5, 6, 7, 8, 9, 10, 14, 15, 28, 30 or more days. In some instances, the antibody is administered in 1 dose, 2 doses, 3 doses, 4 doses, 5 doses, 6 doses or more. In some instances, the antibody is administered at a therapeutically effective amount.

In some instances, the cancer is breast cancer, kidney cancer, lung cancer, ovarian cancer, or colorectal cancer. In some instances, the cancer is a B-cell malignancy.

Disclosed herein, in certain embodiments, are methods of treating cancer in an individual in need thereof, comprising administering to the individual an antibody that selectively binds to a complex comprising an HLA-E and a neoantigen. In some instances, the antibody does not have a binding affinity to (i) the HLA-E alone; or (ii) the neoantigen alone. In some instances, the neoantigen is expressed by an antigen processing machinery (APM)-proficient cell. In some instances, the neoantigen is expressed by a TAP1/2-proficient cell. In some instances, the neoantigen is expressed by an antigen processing machinery (APM)-deficient cell. In some instances, the neoantigen is expressed by a TAP1/2-deficient cell.

In some instances, the neoantigen comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), SEQ ID NO: 31 (VMAPRTLVL), SEQ ID NO: 19 (YLLPRRGPRL), SEQ ID NO: 20 (AISPRTLNA), SEQ ID NO: 21 (SQAPLPCVL), SEQ ID NO: 15 (YLLEMLWRL), SEQ ID NO: 16 (YMLDLQPETT), SEQ ID NO: 22 (QMRPVSRVL), SEQ ID NO: 23 (ALALVRMLI), SEQ ID NO: 24 (SQQPYLQLQ), SEQ ID NO: 25 (AMAPIKTHL), SEQ ID NO: 26 (AMAPIKVRL), SEQ ID NO: 17 (YLLPAIVHI), SEQ ID NO: 27 (ILDQKINEV), SEQ ID NO: 28 (GVYDGEEHSV), SEQ ID NO: 29 (KVLEYVIKV), SEQ ID NO: 18 (SLLMWITQV), SEQ ID NO: 30 (YLEPGPVTV), SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the neoantigen comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), or SEQ ID NO: 31 (VMAPRTLVL). In some instances, the neoantigen comprises, consisting essentially of, or consisting of a sequence according to SEQ ID NO. 3 (VMAPRTLFL).

In some instances, the neoantigen comprises, consisting essentially of, or consisting of a sequence according to SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI). In some instances, the neoantigen comprises, consisting essentially of, or consisting of a sequence according to SEQ ID NO: 35 (MLALLTQVA), SEQ ID NO: 1 (GLADKVYFL), or SEQ ID NO: 2 (ILSPTVVSI). In some instances, the neoantigen comprises, consisting essentially of, or consisting of a sequence according to SEQ ID NO. 35 (MLALLTQVA). In some instances, the neoantigen comprises, consisting essentially of, or consisting of a sequence according to SEQ ID NO. 1 (GLADKVYFL). In some instances, the neoantigen comprises, consisting essentially of, or consisting of a sequence according to SEQ ID NO. 2 (ILSPTVVSI).

In some instances, the HLA-E is HLA-E*0101 or HLA-E*0103. In some instances, the antibody selectively binds to the complex comprising: (a) the HLA-E*0101 and the neoantigen; (b) the HLA-E*1030 and the neoantigen; or (c) the HLA-E*0101 and the neoantigen, and the HLA-E*0103 and the neoantigen.

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), HLA-E and VMAPRTLVL (SEQ ID NO: 31), HLA-E and YLLPRRGPRL (SEQ ID NO: 19), the HLA-E and AISPRTLNA (SEQ ID NO: 20), the HLA-E and SQAPLPCVL (SEQ ID NO: 21), the HLA-E and YLLEMLWRL (SEQ ID NO: 15), the HLA-E and YMLDLQPETT (SEQ ID NO: 16), the HLA-E and QMRPVSRVL (SEQ ID NO: 22), the HLA-E and ALALVRMLI (SEQ ID NO: 23), the HLA-E and SQQPYLQLQ (SEQ ID NO: 24), the HLA-E and AMAPIKTHL (SEQ ID NO: 25), the HLA-E and AMAPIKVRL (SEQ ID NO: 26), the HLA-E and YLLPAIVHI (SEQ ID NO: 17), the HLA-E and ILDQKINEV (SEQ ID NO: 27), the HLA-E and GVYDGEEHSV (SEQ ID NO: 28), the HLA-E and KVLEYVIKV (SEQ ID NO: 29), the HLA-E and SLLMWITQV (SEQ ID NO: 18), the HLA-E and YLEPGPVTV (SEQ ID NO: 30), the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), or HLA-E and VMAPRTLVL (SEQ ID NO: 31). In some instances, the complex comprises the HLA-E and VMAPRTLFL (SEQ ID NO. 3).

In some instances, the complex comprises the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2). In some instances, the complex comprises the HLA-E and MLALLTQVA (SEQ ID NO: 35), the HLA-E and GLADKVYFL (SEQ ID NO: 1), or the HLA-E and ILSPTVVSI (SEQ ID NO: 2). In some instances, the complex comprises the HLA-E and MLALLTQVA (SEQ ID NO. 35). In some instances, the complex comprises the HLA-E and GLADKVYFL (SEQ ID NO. 1). In some instances, the complex comprises the HLA-E and ILSPTVVSI (SEQ ID NO. 2).

In some instances, the antibody is a murine antibody, a chimeric antibody, a camelid antibody, a humanized antibody, or a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody. In some instances, the antibody further comprises a conjugated therapeutic moiety.

In some instances, the selective binding of the antibody to the complex comprising the HLA-E and the neoantigen induces an immune response. In some instances, the immune response comprises activation of T cells. In some instances, the T cell is a CD8+ T cell. In some instances, the immune response comprises activation of cytotoxic T cells (CTL_{S}).

In some instances, the antibody is administered continuously for 1, 2, 3, 4, 5, 6, 7, 8, 9, 10, 14, 15, 28, 30 or more days. In some instances, the antibody is administered at predetermined time intervals for 1, 2, 3, 4, 5, 6, 7, 8, 9, 10, 14, 15, 28, 30 or more days. In some instances, the antibody is administered is administered intermittently for 1, 2, 3, 4, 5, 6, 7, 8, 9, 10, 14, 15, 28, 30 or more days. In some instances, the antibody is administered in 1 dose, 2 doses, 3 doses, 4 doses, 5 doses, 6 doses or more. In some instances, the antibody is administered at a therapeutically effective amount.

In some instances, the cancer is breast cancer, kidney cancer, lung cancer, ovarian cancer, or colorectal cancer. In some instances, the cancer is a B-cell malignancy.

Disclosed herein, in certain embodiments, are methods of producing a camelid antibody that selectively binds to a complex comprising a non-classical HLA-I and a peptide, the method comprising: (a) administering an effective amount of an immunogen to a camelid for eliciting an immune response, wherein the immunogen comprises a recombinantly expressed complex of a non-classical HLA-I and a peptide; (b) constructing an antibody library; (c) assaying the antibody library to select the antibody; and (d) isolating the antibody. In some instances, antibody does not have a binding affinity to (i) the non-classical HLA-I alone; or (ii) the peptide alone. In some instances, the peptide is expressed by an antigen processing machinery (APM)-proficient cell. In some instances, the peptide is expressed by a TAP1/2-proficient cell. In some instances, the peptide is expressed by an antigen processing machinery (APM)-deficient cell. In some instances, the peptide is expressed by a TAP1/2-deficient cell.

In some instances, the peptide comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), SEQ ID NO: 31 (VMAPRTLVL), SEQ ID NO: 19 (YLLPRRGPRL), SEQ ID NO: 20 (AISPRTLNA), SEQ ID NO: 21 (SQAPLPCVL), SEQ ID NO: 15 (YLLEMLWRL), SEQ ID NO: 16 (YMLDLQPETT), SEQ ID NO: 22 (QMRPVSRVL), SEQ ID NO: 23 (ALALVRMLI), SEQ ID NO: 24 (SQQPYLQLQ), SEQ ID NO: 25 (AMAPIKTHL), SEQ ID NO: 26 (AMAPIKVRL), SEQ ID NO: 17 (YLLPAIVHI), SEQ ID NO: 27 (ILDQKINEV), SEQ ID NO: 28 (GVYDGEEHSV), SEQ ID NO: 29 (KVLEYVIKV), SEQ ID NO: 18 (SLLMWITQV), SEQ ID NO: 30 (YLEPGPVTV), SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the peptide comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), or SEQ ID NO: 31 (VMAPRTLVL). In some instances, the peptide comprises, consisting essentially of, or consisting of a sequence according to SEQ ID NO. 3 (VMAPRTLFL).

In some instances, the peptide comprises, consisting essentially of, or consisting of a sequence according to SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI). In some instances, the peptide comprises, consisting essentially of, or consisting of a sequence according to SEQ ID NO: 35 (MLALLTQVA), SEQ ID NO: 1 (GLADKVYFL), or SEQ ID NO: 2 (ILSPTVVSI). In some instances, the peptide comprises, consisting essentially of, or consisting of a sequence according to SEQ ID NO. 35 (MLALLTQVA). In some instances, the peptide comprises, consisting essentially of, or consisting of a sequence according to SEQ ID NO. 1 (GLADKVYFL). In some instances, the peptide comprises, consisting essentially of, or consisting of a sequence according to SEQ ID NO. 2 (ILSPTVVSI).

In some instances, the non-classical HLA-I is HLA-E, HLA-F, HLA-G, or HLA-H. In some instances, non-classical HLA-I is HLA-E. In some instances, the HLA-E is HLA-E*0101 or HLA-E*0103. In some instances, the antibody selectively binds to the complex comprising the HLA-E and the peptide. In some instances, the antibody selectively binds to the complex comprising: (a) the HLA-E*0101 and the peptide; (b) the HLA-E*0103 and the peptide; or (c) the HLA-E*0101 and the peptide, and the HLA-E*0103 and the peptide.

In some instances, the complex comprises the HLA-E and VMAPRTLFL (SEQ ID NO: 3), the HLA-E and VMAPRTLIL (SEQ ID NO: 13), the HLA-E and VMPPRTLLL (SEQ ID NO: 14), the HLA-E and YLLPRRGPRL (SEQ ID NO: 19), the HLA-E and AISPRTLNA (SEQ ID NO: 20), the HLA-E and SQAPLPCVL (SEQ ID NO: 21), the HLA-E and YLLEMLWRL (SEQ ID NO: 15), the HLA-E and YMLDLQPETT (SEQ ID NO: 16), the HLA-E and QMRPVSRVL (SEQ ID NO: 22), the HLA-E and ALALVRMLI (SEQ ID NO: 23), the HLA-E and SQQPYLQLQ (SEQ ID NO: 24), the HLA-E and AMAPIKTHL (SEQ ID NO: 25), the HLA-E and AMAPIKVRL (SEQ ID NO: 26), the HLA-E and YLLPAIVHI (SEQ ID NO: 17), the HLA-E and ILDQKINEV (SEQ ID NO: 27), the HLA-E and GVYDGEEHSV (SEQ ID NO: 28), the HLA-E and KVLEYVIKV (SEQ ID NO: 29), the HLA-E and SLLMWITQV (SEQ ID NO: 18), the HLA-E and YLEPGPVTV (SEQ ID NO: 30), the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO: 3), the HLA-E and VMAPRTLIL (SEQ ID NO: 13), or the HLA-E and VMPPRTLLL (SEQ ID NO: 14). In some instances, the complex comprises the HLA-E and VMAPRTLFL (SEQ ID NO. 3).

In some instances, the complex comprises the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2). In some instances, the complex comprises the HLA-E and MLALLTQVA (SEQ ID NO: 35), the HLA-E and GLADKVYFL (SEQ ID NO: 1), or the HLA-E and ILSPTVVSI (SEQ ID NO: 2). In some instances, the complex comprises the HLA-E and MLALLTQVA (SEQ ID NO. 35). In some instances, the complex comprises the HLA-E and GLADKVYFL (SEQ ID NO. 1). In some instances, the complex comprises the HLA-E and ILSPTVVSI (SEQ ID NO. 2).

In some instances, the antibody is a TCR-like antibody. In some instances, the antibody is a single domain antibody. In some instances, the antibody is a multispecific antibody. In some instances, the antibody is a multifunctional antibody. In some instances, the antibody further comprises a conjugated therapeutic moiety.

In some instances, the selective binding of the antibody to the complex comprising the non-classical HLA-I and the peptide induces an immune response in a cell. In some instances, the immune response comprises activation of T cells. In some instances, the T cell is a CD8+ T cell. In some instances, the immune response comprises activation of cytotoxic T cells (CTL_{S}). In some instances, the cell is a cancer cell.

In some instances, the immunogen is a monomer. In some instances, the immunogen is a tetramer. In some instances, the tetramer comprises avidin or derivatives thereof. In some instances, the immunogen is produced by recombinantly expressing an HLA-I heavy chain and a HLA-I light chain separately in E. coli, and then refolding the HLA-I heavy and light chains with peptide in vitro. In some instances, the camelid is a llama. In some instances, the antibody library is a phage display library. In some instances, the antibody library is a bacteriophage display library. In some instances, the antibody library is a yeast display library. In some instances, the antibody library is a single domain antibody library.

Disclosed herein, in certain embodiments, are pharmaceutical compositions comprising: an antibody disclosed herein; and a pharmaceutically acceptable carrier or excipient.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 exemplifies that protein antigens are processed via the conventional processing route as well as alternative processing routes. Proteins processed via the alternative processing route bind to non-classical HLA-E and to classical HLA class I alleles. Binding of neo-peptides represent true neo-epitopes and provide disease specific targets for immunotherapeutic development.

FIG. 2 exemplifies the clinical and immunological significance of HLA-E in cancer.

FIG. 3 exemplifies TAP-dependent presentation of peptides by HLA-E under physiological conditions, which comprises 5 processing steps for peptide that binds to HLA-E (SEQ ID NOS 56-58 and 31, respectively, in order of appearance).

FIG. 4 exemplifies the structure of leader sequence peptides from MHC class I molecules bound by HLA-E under physiological conditions. Leader sequence binds to HLA-E with amino acids in position 5 and 8 of peptide protruding out from HLA-E peptide pocket.

FIG. 5A-FIG. 5D exemplifies a bispecific scFv with TCR-like targeting and a CD3E binding motif specifically and robustly activates T cells. FIG. 5A is a depiction of a bispecific scFv binding an MHC Class I:peptide complex and activating a proximal T cell. Wells were (1) coated with MHC Class I:peptide monomers, (2) incubated with a distinct bispecific molecule, and (3) co-cultured with naïve T cells that upon activation (4) elaborated IL-2. FIG. 5B exemplifies ELISA detection of IL-2 production by T cells from FIG. 5A. FIG. 5C is a depiction of tumor cells presenting a target bound by a bispecific, which in turn activated a T cell. Wells were provided (1) EL4 tumor cells expressing a specific MHC Class I:peptide, (2) bispecific therapeutic, and (3) antigen-naïve T cells. FIG. 5D exemplifies ELISA detection of IL-2 production by T-cells from FIG. 5C.

FIG. 6 exemplifies a validation strategy used for cancer specific HLA-E-peptide targets. 1) Snap-frozen tumor tissue isolated from patient-derived xenograft (PDX) models, patient biopsies or freshly isolated hematological cancer cells are stored frozen in liquid nitrogen. 2) Sample of material is isolated and resuspended in 5 to 10 ml of lysis buffer and homogenized for 10 seconds on ice. After 1 hr incubation on ice, the sample is centrifuged for 20 min at 40,000 g. 3) An affinity column is prepared using antibody 4D12 or another antibody to HLA-E. The purified anti-HLA-E antibodies are coupled to a CN-Br-activated sepharose beads. The clarified supernatant is then applied to the affinity column. The column is washed with PBS, followed by a second wash with water and the sample eluted with 0.1 M glycine pH 3.0. 4) Collected sample is immediately neutralized with addition of NH₄HCO₃. Removal of heavy chain and beta-2-microglobulin (B2M) is performed using a 5 kDa filtration membrane and smaller molecular peptides pass through membrane and are collected for 5) analysis on LC/MS/MS (ThermoFisher Orbitrap). 6) Synthetic peptides are purified on LC/MS/MS and compared to in silico discovered peptide profile to validate presence of tumor specific peptide target (SEQ ID NO: 59).

FIG. 7A-FIG. 7C exemplifies LC/MS/MS validation profile of peptide GLADKVYFL (SEQ ID NO: 1) isolated from HLA-E molecules expressed in PDX lung tumor tissue. FIG. 7A illustrates LC retention time of GLADKVYFL (SEQ ID NO: 1) peptide. FIG. 7B illustrates the Mass/Charge ratio of the GLADKVYFL (SEQ ID NO: 1) peptide and FIG. 7C aligns the MS fragmentation profile of the synthetic peptide standard with peptide GLADKVYFL (SEQ ID NO: 1) isolated from HLA-E from PDX lung cancer sample.

FIG. 8A-FIG. 8C exemplifies LC/MS/MS validation profile of peptide ILSPTVVSI (SEQ ID NO: 2) isolated from HLA-E molecules expressed in PDX lung tumor tissue. FIG. 8A illustrates LC retention time of ILSPTVVSI (SEQ ID NO: 2) peptide. FIG. 8B illustrates the Mass/Charge ratio of the ILSPTVVSI (SEQ ID NO: 2) peptide and FIG. 8C aligns the MS fragmentation profile of the synthetic peptide standard with peptide ILSPTVVSI (SEQ ID NO: 2) isolated from HLA-E from PDX lung cancer sample.

FIG. 9A-FIG. 9D exemplifies production and characterization of recombinant HLA-E*0101-VMAPRTLFL (HLA-G signal peptide) protein. FIG. 9A illustrates separation profile of resulting products from an HLA-E*0101 refold using peptide VMAPRTLFL (SEQ ID NO: 3). Refolded protein material was run on an FPLC Superdex 75 column (GE) using the NGC™ Medium-Pressure Liquid Chromatography System (Bio-Rad). Second peak, stated as refold peak, contains correctly recombined and functional HLA-E*0101-VMAPRTLFL complex. FIG. 9B illustrates a coomassie blue stained gel that reveals HLA-E heavy chain (33 kD) and beta-2-microglobulin (11 kD) bands from peak 2 (FIG. 9A) after being run on a 12% SDS-polyacrylamide gel in lane designated as b-V-0025-E(0101). FIG. 9C illustrates HPLC (Shimadzu 2020) profile with 10 mg of peak 2 (FIG. 9A) run on a Waters Xbridge BEH size exclusion column. Expected retention time of 6.384 minutes was confirmed supporting presence of properly refolded HLA-E peptide complex. FIG. 9D illustrates 3D12 antibody (10 μg/ml) binding to immobilized biotin-labeled HLA-E*0101-VMAPRTLFL (peak 2 FIG. 9A). In brief, a bionetic plate (Resonant Sensors) was coated with neutravidin (10 μg/ml) to capture biotin-labeled HLA-E-peptide complex. Binding of conformational dependent 3D12 antibody to HLA-E-peptide complex was determined using the ResoSens instrument (Resonant Sensors) as pico-meter shift over time (min).

FIG. 10A-FIG. 10D exemplifies production and characterization of recombinant HLA-E*0103-VMAPRTLFL (HLA-G signal peptide) protein. FIG. 10A illustrates separation profile of resulting products from an HLA-E*0103 refold using peptide VMAPRTLFL (SEQ ID NO: 3). Refolded protein material was run on an FPLC Superdex 75 column (GE) using the NGC^{TM} Medium-Pressure Liquid Chromatography System (Bio-Rad). Second peak, stated as refold peak, contains correctly recombined and functional HLA-E*0103-VMAPRTLFL complex. FIG. 10B illustrates a coomassie blue stained gel that reveals HLA-E heavy chain (33 kD) and beta-2-microglobulin (11 kD) bands from peak 2 (FIG. 10A) run on a 12% SDS-polyacrylamide gel in lane designated as b-V-0025-E(0103). FIG. 10C shows HPLC (Shimadzu 2020) profile with 10 μg of peak 2 (FIG. 10A) run on a Waters Xbridge BEH size exclusion column. Expected retention time of 6.384 minutes was confirmed supporting presence of properly refolded HLA-E peptide complex. FIG. 10D shows 3D12 antibody (10 μg/ml) binding to immobilized biotin-labeled HLA-E*0103-VMAPRTLFL (peak 2 FIG. 10A). In brief, a bionetic plate (Resonant Sensors) was coated with neutravidin (10 μg/ml) to capture biotin-labeled HLA-E-peptide complex. Binding of conformational dependent 3D12 antibody to HLA-E-peptide complex was determined using the ResoSens instrument (Resonant Sensors) as pico-meter shift over time (min).

FIG. 11A-FIG. 11D exemplifies production and characterization of recombinant HLA-E*0103-GLADKVYFL (CAD protein). FIG. 11A illustrates separation profile of resulting products from an HLA-E*0103 refold using peptide GLADKVYFL (SEQ ID NO: 1). Refolded protein material was run on an FPLC Superdex 75 column (GE) using the NGC™ Medium-Pressure Liquid Chromatography System (Bio-Rad). Second peak, stated as refold peak, contains correctly recombined and functional HLA-E*0103-GLADKVYFL complex. FIG. 11B illustrates a coomassie blue stained gel that reveals HLA-E heavy chain (33 kD) and beta-2-microglobulin (11 kD) bands from peak 2 (FIG. 11A) after being run on a 12% SDS-polyacrylamide gel in lane designated as b-V-0011-E. FIG. 11C illustrates HPLC (Shimadzu 2020) profile with 10 μg of peak 2 (FIG. 11A) run on a Waters Xbridge BEH size exclusion column. Expected retention time of 6.384 minutes was confirmed supporting presence of properly refolded HLA-E peptide complex. FIG. 11D illustrates 3D12 antibody (10 μg/ml) binding to immobilized biotin-labeled HLA-E*0103-GLADKVYFL (peak 2 FIG. 11A). In brief, a bionetic plate (Resonant Sensors) was coated with neutravidin (10 μg/ml) to capture biotin-labeled HLA-E-peptide complex. Binding of conformational dependent 3D12 antibody to HLA-E-peptide complex was determined using the ResoSens instrument (Resonant Sensors) as pico-meter shift over time (min).

FIG. 12A-FIG. 12D exemplifies production and characterization of recombinant HLA-E*0103-ILSPTVVSI (KIF11 protein). FIG. 12A illustrates separation profile of resulting products from an HLA-E*0103 refold using peptide ILSPTVVSI (SEQ ID NO: 2). Refolded protein material was run on an FPLC Superdex 75 column (GE) using the NGC^{TM} Medium-Pressure Liquid Chromatography System (Bio-Rad). Second peak, stated as refold peak, contains correctly recombined and functional HLA-E*0103-ILSPTVVSI complex. FIG. 12B illustrates a coomassie blue stained gel that reveals HLA-E heavy chain (33kD) and beta-2-microglobulin (11 kD) bands from peak 2 (FIG. 12A) after being run on a 12% SDS-polyacrylamide gel in lane designated as b-V-00013-E. FIG. 12C illustrates HPLC (Shimadzu 2020) profile with 10 μg of peak 2 (FIG. 12A) run on a Waters Xbridge BEH size exclusion column. Expected retention time of 6.384 minutes was confirmed supporting presence of properly refolded HLA-E peptide complex. FIG. 12D illustrates 3D12 antibody (10 μg/ml) binding to immobilized biotin-labeled HLA-E*0103-ILSPTVVSI (peak 2 FIG. 12A). In brief, a bionetic plate (Resonant Sensors) was coated with neutravidin (10 μg/ml) to capture biotin-labeled HLA-E-peptide complex. Binding of conformational dependent 3D12 antibody to HLA-E-peptide complex was determined using the ResoSens instrument (Resonant Sensors) as pico-meter shift over time (min).

FIG. 13 is an exemplary schematic of the antibody discovery cycle used to generate high affinity antibodies. FIG. 13 discloses SEQ ID NO: 60.

FIG. 14A-FIG. 14D exemplifies discovery of antibodies to HLA-E-VMAPRTLFL. FIG. 14A illustrates a single clone discovered from a naïve semi-synthetic human antibody library displayed by bacteriophage. Four rounds of selection were used to identify the highly specific clone. For rounds 1-3, blocking and depletion with HLA-A2 negative targets was performed followed by positive selection using HLA-E-VMAPRTLFL. The 4^{th} round of selection involved blocking and depletion with stringent HLA-E negative target (HLA-E-YLLPAIVHL) followed by positive selection. FIG. 14B illustrates 6 unique clones isolated from an immunized mouse phage display library. In brief, Balb/c mice were immunized with 50 μg of HLA-E-VMAPRTLFL protein 3× at 2 week intervals. After a final injection via tail vein, the spleen from a single mouse was harvested and total RNA was isolated for cDNA synthesis and library construction. Clone selection followed similar design as described in FIG. 14A for the human antibody library. FIG. 14C illustrates the PCR amplification results for constructing of a VHH single domain library from an immunized llama. FIG. 14D illustrates 15 VHH antibody clones to HLA-E-VMAPRTLFL isolated from an immunized llama. In brief, a llama was immunized weekly with 100 μg of tetramerized HLA-E-VMAPRTLFL complex for 6 weeks. After determining final titer, blood was removed and B cells isolated for harvesting total RNA. A single-domain library was constructed for antibody display in phage. Selection followed protocol described in FIG. 14A and FIG. 14B.

FIG. 15 illustrates results from a monoclonal phage ELISA for specific binding of murine scFv clones to HLA-E-ILSPTVVSI peptide complex from an immunized library. In brief, a Balb/c mouse was immunized with 50 μg of HLA-E*0103-ILSPTVVSI peptide complex three times at 2 week intervals followed by a final 10 μg of antigen injection via tail vein. Four days later, the spleen was harvested and total RNA was isolated to synthesize cDNA. VH and VL genes were amplified from cDNA templates using primers and scFv genes were generated by overlapping PCR for cloning into the phagemid vector. Ligated scFv genes in phagemid were electro-transformed into TG1 competent E. coli cells to make the end library. The phage displayed scFv proteins were packaged with the aid of helper phage M13KO7 using standard methods. The library showed 30 of 30 clones carried scFv insertion and the diversity of the library was 5.5×10⁸. After 3 rounds of selection, 40 clones were submitted for DNA sequencing with a total of 6 unique clones being identified. The 6 scFv phage clones were grown and tested by ELISA for specific binding to target antigen HLA-E-ILSPTVVSI.

FIG. 16A-FIG. 16C illustrate binding specificity of a yeast library displaying murine scFv after 4 rounds of enrichment. FIG. 16A exemplifies binding preference of a yeast display library for 1 μM of specific target HLA-E-ILSPTVVSI. Events in gated area (boxed in Q2) represent yeast binding the target HLA-E-ILSPTVVSI and were sorted using a FACS Aria II sorter. Recovered yeast were expanded and scFv expression induced before staining again with antigen as shown in FIG. 16B. Using 100 nM of antigen, yeast display library shows binding only to the specific target (1 nM) of HLA-E-ILSPTVVSI. FIG. 16C illustrates that Clone 3 shows significant staining of A549 TAP1 K/O cells. Purified Clone 3, human IgG1 that binds to HLA-E-ILSPTVVSI complex, was used at 1 ug/ml to stain A549 and A549 TAP1 K/O cells.

FIG. 17A-FIG. 17C exemplifies binding specificity for R4 human antibody clone to the HLA-E*0103-VMAPRTLFL complex. FIG. 17A illustrates scFv R4 human antibody expression in E. coli and binding specificity for HLA-E-VMAPRTLFL target at both 50 nM and 5 nM concentration by ELISA. Produced scFv protein was purified on a NiNTA column and 5 μg of purified sample was run on a 12% SDS-PAGE gel under reducing conditions. Coomassie blue staining revealed a single band at the correct size of ˜30 kD). FIG. 17B illustrates expression and specific binding of full-length IgG1 R4 human antibody. R4 IgG1 was expressed in HEK-293 cells and purified on a Protein-A column. Antibody was run on a 12% SDS-PAGE under reducing conditions and stained with Coomassie blue. The destained gel revealed two dominant bands at the correct size of ˜50 kD and 25 kD. R4 IgG1 was used in an ELISA at various concentrations (0, 0.5, 1 and 4 nM) to determine specificity of binding to target complex (HLA-E-VMAPRTLFL). FIG. 17C illustrates the binding kinetics and affinity constant for R4 clone (scFv format) using Octet (ForteBio) and standard protocol.

FIG. 18 exemplifies preliminary epitope mapping of R4 IgG1 human antibody binding specificity targeting the HLA-E*0103-VMAPRTLFL complex using ResoSens label-free technology. In brief, biotin-labeled monomers of HLA-E produced with different peptides were captured on bionetic plates containing neutravidin. The peptides used to make HLA-E peptide complexes include the following sequences: VMAPQALLL (SEQ ID NO: 4) (ABI-V-0040), VMAPRTLLL (SEQ ID NO: 5) (ABI-V-0042), VMAPRTLTL (SEQ ID NO: 6) (ABI-V-0043), VMAPRTVLL (SEQ ID NO: 7) (ABI-V-0044), VTAPRTVLL (SEQ ID NO: 8) (ABI-V-0046), VMAPRTLYL (SEQ ID NO: 9) ((ABI-V-0047), VMAPRTLWL (SEQ ID NO: 10) (ABI-V-0048), and VMAPRTLFL (SEQ ID NO: 3) (ABI-V-0025). Peptides ABI-V-0047 and ABI-V-0048 are not found in nature and were used as controls. R4 IgG1 antibody was run on a bionetic plate using the ResoSens instrument. Antibody binding (y-axis) to HLA-E-peptide complexes determined before washing (pre-wash binding) and after washing (post-wash binding). R4 IgG1 exhibits fine binding specificity for HLA-E-VMAPRTLFL and peptides VMAPRTLYL (SEQ ID NO: 9) and VMAPRTLWL (SEQ ID NO: 10) having highly conserved amino acid residues containing aromatic ring structures in p8.

FIG. 19A-FIG. 19B exemplifies R4 IgG1 human antibody binding to both HLA-E*0101-VMAPRTLFL and HLA-E*0103-VMAPRTLFL complexes using ResoSens label-free technology. In brief, biotin-labeled monomers of HLA-E*0101 and *0103 loaded with VMAPRTLFL (SEQ ID NO: 3) peptide were captured on a bionetic plate containing neutravidin. FIG. 19A illustrates R4 IgG1 antibody (10 μg/ml) binding to HLA-E*0101-VMAPRTLFL. FIG. 19B illustrates R4 IgG1 antibody (10p g/m1) binding to HLA-E*0103-VMAPRTLFL complex. Pre-wash and Post-wash binding with R4 IgG4 antibody reveal similar on and off rates and total resonant shift units (pMeter) indicating R4 antibody shows similar binding preference for both HLA-E alleles presenting the VMAPRTLFL (SEQ ID NO: 3) peptide.

FIG. 20 exemplifies staining of tumor cells with mouse IgG1 antibody 3D12 (anti-HLA-E, top panel) and R4 IgG1 human antibody (bottom panel). As indicted, top panel shows staining with 3D12 and bottom panel shows staining with R4 antibody (used at 1 μg/ml). As indicated top and bottom panel show staining of tumor cells with isotype control antibody (mouse IgGland human IgG1), respectively. Detection of primary antibody binding was determined by flow cytometric analysis using an LSR FACS analyzer (BD) and staining with secondary goat anti-mouse IgG-FITC for 3D12 and mouse isotype control (top panel) and secondary goat anti-human IgG-APC for R4 and human isotype control antibody (bottom panel).

FIG. 21A-FIG. 21C exemplifies staining of tumor cells with 3D12, anti-HLA-E and R4 IgG1, anti-HLA-E-VMAPRTLFL antibody. FIG. 21A illustrates human colorectal cell line, HCT-116 expressing TAP1 protein or lacking TAP1 protein (TAP1 gene K/O) treated with IFN-g for 48 hrs and stained with 3D12 and R4 antibody at lug/ml. Primary antibody binding was detected by FACS (LSR, BD) using secondary goat anti-mouse antibody-FITC conjugate (top panel) or with secondary goat-anti-human antibody-APC conjugate (bottom panel). FIG. 21B illustrates human NSCLC cell line, A-549 expressing TAP1 protein or lacking TAP1 protein (TAP1 gene K/O) and stained with 3D12 and R4 antibody at 1 μg/ml. Primary antibody binding was detected by FACS (LSR, BD) using secondary goat anti-mouse antibody-FITC conjugate (top panel) or with secondary goat-anti-human antibody-APC conjugate (bottom panel). The VMAPRTLFL (SEQ ID NO: 3) peptide binding to HLA-E is dependent on the presence of TAP1 protein. R4 antibody stains both TAP positive HCT-116 and A-549 cell lines but not cell lines lacking TAP1 protein. FIG. 21C illustrates time course expression profile of HLA-G protein in cell lines HCT-116 and A-549 with IFN-gamma treatment. Cell lysates prepared and run on 12% SDS-PAGE gel. After completion of electrophoresis, samples were transferred to nitrocellulose membrane and probed with anti-HLA-G antibody. An antibody to B-actin protein was used as a loading control.

FIG. 22 exemplifies broad expression of HLA-E protein in human tumor tissue.

FIG. 23 exemplifies anti-HLA-E antibody staining of human ovarian cancer samples. The data indicate MEM-E0/2anti-HLA-E antibody stains ovarian tumor tissues (n=48). Approximately 90% of tumor samples stained were positive for HLA-E expression with 60% of tumors showing high to medium HLA-E protein expression.

FIG. 24 exemplifies HLA-E expression in human colorectal cancer tissues (n=48). More than 90% of human colorectal tumors showed positive staining with the anti-HLA-E antibody, MEM-E0/2. Further, approximately 65% of tumors had high to medium HLA-E protein expression.

FIG. 25A-FIG. 25B exemplifies a representative staining pattern using MEM-E/02 antibody to detect HLA-E protein in human cancers. FIG. 25A illustrates membrane staining of HLA-E protein in human breast tumor tissue. FIG. 25B illustrates detection of HLA-E protein on membrane and in cytoplasm in human breast cancer tissue.

FIG. 26 exemplifies a schematic of a strategy to leverage HLA-E-peptide targets to redirect the immune system towards tumors for destruction and elimination.

FIG. 27A-FIG. 27F exemplify that HLA-E-peptide complexes represent novel druggable targets for oncology applications. FIG. 27A illustrates a representative bispecific antibody T cell engager (BiTE) format used for targeting HLA-E-peptide complexes for tumor cell destruction. The R4 antibody that recognizes HLA-E-VMAPRTLFL peptide complex, was cloned as an VH linker VL scFv molecule and covalently linked via a (GGGS)4 linker (SEQ ID NO: 11) to the VL-VH scFv from OKT3, an anti-human CD3 antibody. The C-terminal end of the BiTE contained a 6-his tag (SEQ ID NO: 12) for downstream purification and detection. FIG. 27A discloses SEQ ID NOS 61-62, 61, and 12, respectively, in order of appearance. FIG. 27B illustrates coomassie blue staining and Western blot analysis of NiNTA chromatography enriched BiTE 86-2. FIG.27C illustrates purified 86-2 BiTE stains CD3 marker on T-lymphocytes and HLA-E-VMAPRTLFL target on Colo205 cancer cells indicated by red peak shifting to the right of blue peak. FIG. 27D illustrates IL-2 production of T cells addition of BiTE 86-2 to culture containing Jurkat T cells and COLO205 tumor cells. In the absence of BiTE 86-2, little IL-2 cytokine is detected. After addition of BiTE to culture wells, the functional BiTE molecule binds to CD3 on Jurkat cells and HLA-E-VMAPRTLFL peptide on tumor cells inducing the activation and production of IL-2 by Jurkat cells. FIG. 27E illustrates PBMCs +BiTE 86-2 mediate COLO205 tumor cell killing (20.2%) compared to tumor cytotoxicity in control group (without BiTE molecule 7.41%). FIG. 27F illustrates dose-dependent redirected CD8+ T cell cytotoxicity (reduced viability) of NCIH-1563 lung cancer cells treated with BiTE 86-2 molecule.

### DETAILED DESCRIPTION OF THE DISCLOSURE

Disclosed herein, in certain embodiments, are antibodies that selectively bind to a complex comprising a non-classical HLA-I and a neoantigen. Further disclosed herein, in certain embodiments, are methods of treating a cancer by administering an antibody that selectively binds to a complex comprising a non-classical HLA-I and a neoantigen. In some embodiments, the antibodies that selectively bind to a complex comprising a non-classical HLA-I and a neoantigen modulate immune response against cancer cells, thereby treating cancer.

Traditional approaches to the treatment of cancers have included surgery, radiation, chemotherapy and hormone therapy. However, such therapies have not proven effective by themselves. Development of alternate remedies for preventing and/or treating cancer is crucial. More recently immunotherapy and gene therapy approaches utilizing antibodies and T-lymphocytes have emerged as new and promising methods for treating cancer.

Major histocompatibility complex (MHC) molecules, designated human leukocyte antigen (HLA) in humans, play a critical role in the body's recognition of disease and the resulting immune response to cancer and invading antigens. The HLA gene family is divided into two subgroups namely HLA Class I (HLA-I) and HLA Class II (HLA-II), with HLA-I further divided into classical HLA-I and non-classical HLA-I. Each HLA molecule forms a complex with one peptide from within the cell. On cancer cells, some of the peptide/HLA complexes are uniquely presented which enables the immune system to recognize and kill these cells. Cells decorated with these unique peptide/HLA complexes are recognized and killed by the cytotoxic T cells (CTLs). Cancer cells show a downregulation in classical HLA-I expression but an upregulation in non-classical HLA-I expression (e.g. HLA-E). Thus, the upregulated uniquely presented non-classical HLA-I-peptide complexes on cancer cells are novel targets for developing innovative immunotherapies for treatment of cancer.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, the term “MHC” refers to the Major Histocompability Complex, which is a set of gene loci specifying major histocompatibility antigens. The term “HLA” as used herein refer to Human Leukocyte Antigens, which are the histocompatibility antigens found in humans. As used herein, “HLA” is the human form of “MHC” and the terms are used interchangeably.

As used herein “antibody” refers to a glycoprotein which exhibits binding specificity to a specific antigen. Antibodies herein also include “antigen binding portion” or fragments of the antibody that are capable of binding to the antigen. The term includes, but is not limited to, polyclonal, monoclonal, monospecific, multispecific (e.g., bispecific antibodies), natural, humanized, human, chimeric, synthetic, recombinant, hybrid, mutated, grafted, antibody fragments (e.g., a portion of a full-length antibody, generally the antigen binding or variable region thereof, e.g., Fab, Fab′, F(ab′)2, and Fv fragments), and in vitro generated antibodies so long as they exhibit the desired biological activity. The term also includes single chain antibodies, e.g., single chain Fv (sFv or scFv) antibodies, in which a variable heavy and a variable light chain are joined together (directly or through a peptide linker) to form a continuous polypeptide.

As used herein, the term “selectively binds” in the context of any binding agent, e.g., an antibody, refers to a binding agent that binds specifically to an antigen or epitope, such as with a high affinity, and does not significantly bind other unrelated antigens or epitopes.

As used herein the term “neoantigen” or “neopeptide” are used interchangeably and refer to a peptide expressed by a diseased or stressed cell (e.g. cancer cell).

As used herein, the term “immunogen” refers to a moiety, which optionally can be administered to a subject, which induces an immunological response.

The terms “recipient”, “individual”, “subject”, “host”, and “patient”, are used interchangeably herein and in some cases, refer to any mammalian subject for whom diagnosis, treatment, or therapy is desired, particularly humans. None of these terms require the supervision of medical personnel.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with diseases (e.g. cancer). The term “therapeutic effect” refers to the reduction, elimination, or prevention of the disease, symptoms of the disease, or side effects of the disease in the subject.

### Major Histocompability Complex (MHC) or Human Leukocyte Antigens (HLA)

Major histocompatibility complexes (MHC), also termed Human Leukocyte Antigens (HLA) in humans are glycoproteins expressed on the surface of nucleated cells that act as proteomic scanning chips by providing insight into the status of cellular health. They continuously sample peptides from normal host cellular proteins, cancer cells, inflamed cells and bacterial, viral and parasite infected cells and present short peptides on the surface of cells for recognition by T lymphocytes. Presented peptides can also be derived from proteins that are out of frame or from sequences embedded in the introns, or from proteins whose translation is initiated at codons other than the conventional methionine codon, ATG.

There are two classes of MHCs in mice and humans, namely MHC I and MHC II. MHC I comprises classical and non-classical MHC I sub groups.

### Classical Major Histocompatibility Complex I (MHC I) or HLA-I

Classical MHC I molecules include HLA-A, HLA-B and HLA-C in humans and H-2-K, H-2-D, H-2-B and H-2-L in mice. Classical MHC I molecules are highly polymorphic with more than 2,735 alleles of HLA-A, 3,455 alleles of HLA-B and 2,259 alleles of HLA-C. Classical MHC I is expressed on the surface of all nucleated cells and present peptides to CD8 T lymphocytes. 30% of the proteins in the cellular machinery are rapidly degraded and are primary substrates for classical MHC I antigen presentation.

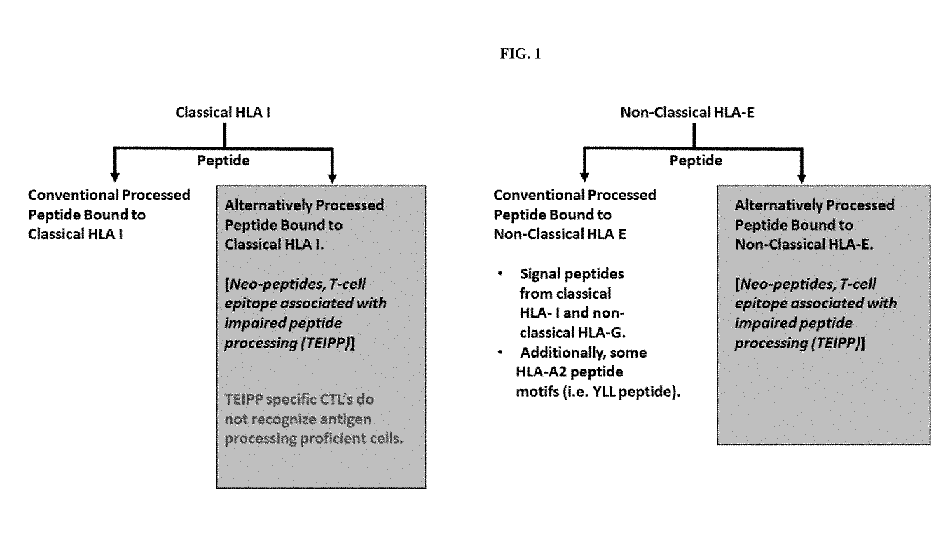

For peptide to be presented by classical MHC I molecules, proteins are first processed through the conventional processing route (ubiquitin proteasome system) that begins with the proteasome. The breakdown products (2 to 25 amino acid residues) in length are released into the cytosol. Selected cytosolic peptides are then transported into endoplasmic reticulum via Transporter associated protein (TAP) complex. TAP belongs consists of heterodimeric subunits, TAP1 and TAP2, and both bind to a transmembrane adapter chaperon glycoprotein called tapasin. Endoplasmic reticulum amino peptidase (ERAAP) in the endoplasmic reticulum trims amino-terminally extended precursors delivered by TAP to generate peptides of 8-10 amino acids in length that load onto classical MHC I molecules. Thus, the conventional processing route begins with protein degradation in the proteasome and TAP dependent transport of peptides into the endoplasmic reticulum (ER) and ends with the loading of peptides into the HLA peptide binding pocket (FIG. 1). The proteins that contribute to the conventional processing route are collectively known as antigen processing machinery (APM) and include the proteasome, Transporter associated protein (TAP) complex, tapasin, endoplasmic reticulum amino peptidase (ERAAP), binding immunoglobulin protein (BiP), clanexin and calreticulin. Cells lacking either proteasome subunits, TAP1/2, ErP57 or calreticulin have reduced numbers of classical MHC I molecules on their surface.

### Non-Classical MHC I or HLA-I

Non-classical MHC I molecules include HLA-E, HLA-F and HLA-G, and have limited polymorphisms. They play a role in regulating innate and adaptive immune responses. Non-classical MHC I molecules present peptides generated by both the conventional processing route and the alternative processing route in health and disease states, and represent a novel set of markers for targeting in disease states (e.g. cancer).

### HLA-E

The non-classical MHC class I molecule, HLA-E is non-polymorphic. In nature, 13 HLA-E alleles have been identified with only two functional variants, namely HLAE* 0101 and HLA-E*0103. The difference between HLA-E*0101 (HLA E^{107′}) and *0103 (HLA-E^{107G} is a single amino acid difference at position 107 which is outside the peptide binding pocket. Similar to the classical MHC I molecules, HLA-E is expressed in all cells with a nucleus, however at usually lower levels. HLA-E molecule expression in cells and tissues is generally increased during stress and disease.

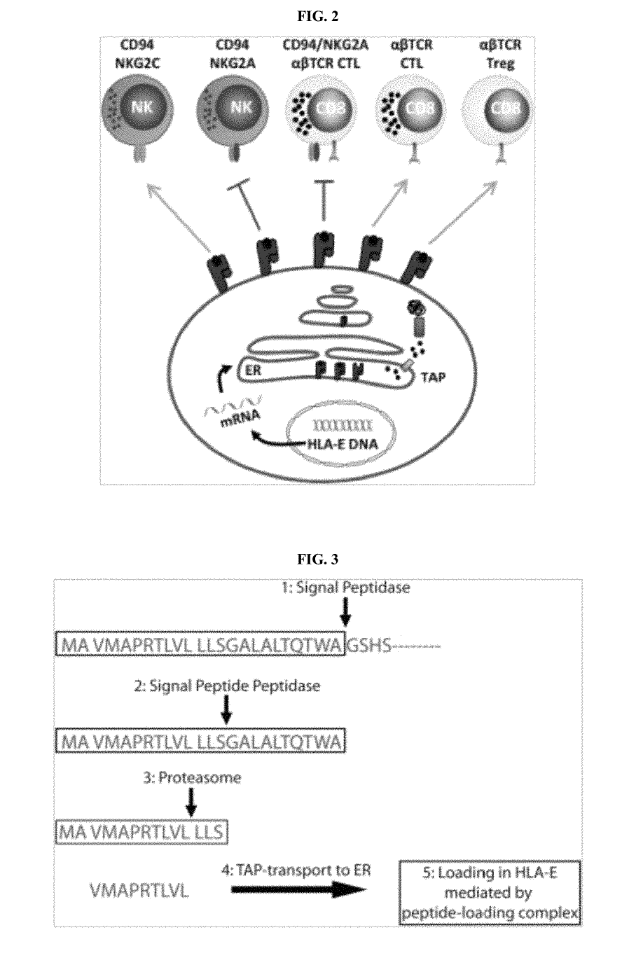

In healthy cells, HLA-E presents peptides derived from classical MHC molecules and the non-classical HLA-G molecule to either inhibit or stimulate the activity of NK cells and a subset of CD8 T cells through engaging the receptor CD94/NKG2 (FIG. 2). Depending on the particular peptide presented by HLA-E, the HLA-E complex will engage either CD94/NKG2A or CD94/NKG2C to inhibit or activate NK cells and a subset of CD8 T cells, respectively.

Peptides derived from classical MHC I molecules are generated in a 5 step process that starts with signal peptidases cleaving the signal peptide from the full-length protein (FIG. 3). The released signal peptide is further trimmed by a specific signal peptide peptidase before being transported to the proteasome for additional trimming. In step 4, the peptide, generally a nanomer, is transported to the lumen of the endoplasmic reticulum by TAP 1 and TAP 2 wherein the successfully transported signal peptide is loaded in HLA-E by a set of defined chaperones within the lumen of the ER. An example of HLA-E peptide binder derived from classical HLA's is HLA-Cw*02 (VMAPRTLLL (SEQ ID NO: 5)). Subtle changes in peptide conformation affect recognition of the HLA-E-peptide complex by the CD94/NKG2 Natural Killer cell receptors.

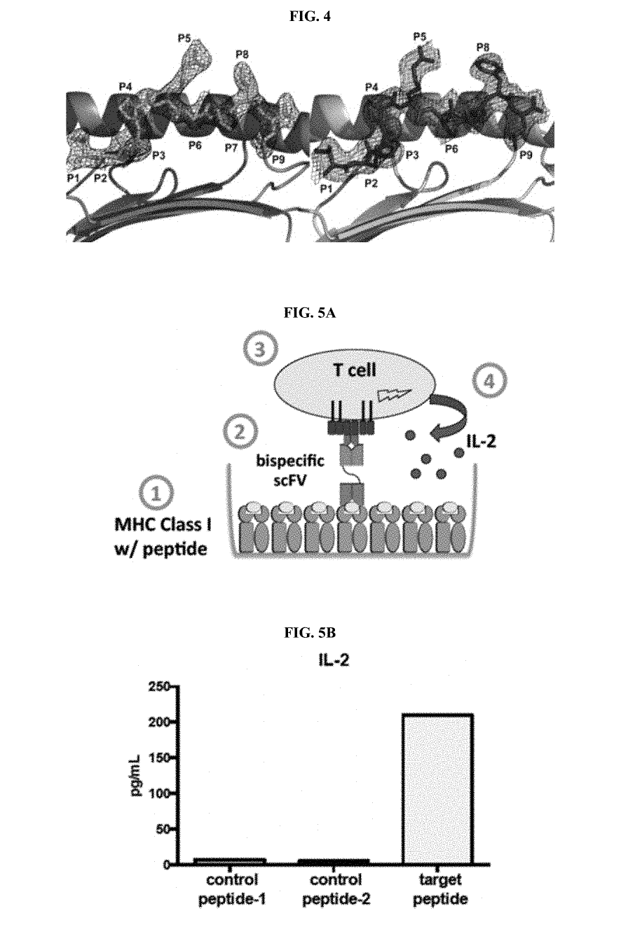

In healthy cells, HLA-E binds peptides that are generally 9 to 11 amino acids in length and exhibit a high degree of hydrophobicity Unlike peptides that bind to classical MHC I molecules that usually have 2 or 3 anchor residues within the peptide sequence, non-classical HLA-E binds peptides through interaction via 5 anchor positions, namely p2, 3, 6, 7 and 9 (FIG. 4). Peptide complexes bound to HLA-E show amino acids at P5 and P8 protruding out from the binding pocket. Moreover, because more residues of the peptide are anchor peptides, the binding pocket of HLA-E with peptide binding has several deep pockets that may be targeted by small highly specific binding molecules. In contrast, the two protruding amino acids (p5 and p8) interact with CD94/NKG2 receptors on both NK cells and a subset of CD8+ T cells. Further examples of peptides include VMAPRTLIL (SEQ ID NO: 13), peptide from HLA-C×03 and VMPPRTLLL (SEQ ID NO: 14), peptide from HLA-B*8001.

Another signal peptide that has characteristics in common with signal peptides generated from classical HLA-I molecules is the signal peptide generated from non-classical HLA-G. HLA-G expression under normal physiologic conditions is tightly regulated, with limited expression found in relatively few tissues and cells in the body. HLA-G plays a key role as an immune tolerant molecule and its expression is observed in cancer tissue/cells. Moreover, the signal peptide from HLA-G is processed by the conventional antigen processing pathway and delivered to the endoplasmic reticulum by the peptide transporter TAP. In some instances, the signal peptide is VMAPRTLFL (SEQ ID NO: 3).

### HLA-E Expression and Peptide Presentation in Cancer Cells

Cells deficient in one or more components of the antigen processing machinery (APM) (e.g. proteasome, tapasin, or TAP) load peptides into MHC class I molecules via alternative processing routes which are independent of the APM-dependent conventional processing route. APM-deficient cells not only have reduced numbers of classical MHC I molecules on their surface, but also show an increase in the cell surface density of HLA-E molecules as well as an increase in the repertoire of peptides presented. The alternative processing routes are constitutively turned on and produce peptides in both healthy and diseased cells. These peptides, however, are not presented by healthy cells; instead they are only presented in diseased or stressed cells. As such, the different peptide repertoires generated by APM-defective cells, also known as “T-cell epitopes associated with impaired peptide processing” (TEIPP), represent novel targets unique to cancer cells, and represent ideal targets for therapeutic development in the treatment of cancer.

HLA-E presents TEIPP during cellular stress, i.e. infection or cancer, (Table 1). A few of these HLA-E binding peptides are identified as having HLA-A*0201 and HLA-Cw2 binding motifs. The four HLA-A*0201 peptides binders that also bind to HLA-E include EBV LMP1 peptide YLLEMLWRL (SEQ ID NO: 15), HPV peptide YMLDLQPETT (SEQ ID NO: 16), host protein RNA Helicase p68 peptide YLLPAIVHI (SEQ ID NO: 17) and the classical tumor antigen peptide NY-ESO-1 peptide SLLMWITQV (SEQ ID NO: 18).

TABLE 1
Peptide binders for HLA-E identified in TAP-deficient tumor cells.

| Viral peptides shown to bind HLA-E | | SYFPEITHI Score HLA-A2 Max 36 |
|---|---|---|
| HCV Core | YLLPRRGPRL (SEQ ID NO: 19) | 27 |
| HIV gag protein | AISPRTLNA (SEQ ID NO: 20) | 19 |
| EBV BZLF-1 | SQAPLPCVL (SEQ ID NO: 21) | 17 |
| protein | YLLEMLWRL (SEQ ID NO: 15) | 30 |
| EBV LMP1 | YMLDLQPETT (SEQ ID NO: 16) | 19 |
| HPV | | |
| Self peptides shown to bind HLA-E | | SYFPEITHI Score HLA-A2 |
| Hsp60 | QMRPVSRVL (SEQ ID NO: 22) | 20 |
| MRP7 | ALALVRMLI (SEQ ID NO: 23) | 22 |
| Gliadin | SQQPYLQLQ (SEQ ID NO: 24) | 9 |
| Prdx5delta | AMAPIKTHL (SEQ ID NO: 25) | 24 |
| Prdx5delta | AMAPIKVRL (SEQ ID NO: 26) | 26 |
| RNA Helicase p68 | YLLPAIVHI (SEQ ID NO: 17) | 30 |
| ODC | ILDQKINEV (SEQ ID NO: 27) | 30 |
| Clinical tumor antigen peptides shown to bind HLA-E | | SYFPEITHI Score HLA-A2 |
| MAGE-B2 | GVYDGEEHSV (SEQ ID NO: 28) | 20 |
| Classical tumor antigens binding to HLA-E (in vitro data only) | | SYFPEITHI Score HLA-A2 |
| MAGE-A1 | KVLEYVIKV (SEQ ID NO: 29) | 26 |
| NY-ESO-1 | SLLMWITQV (SEQ ID NO: 18) | 28 |
| Gp100 | YLEPGPVTV (SEQ ID NO: 30) | 29 |

### MHC II or HLA-II

MHC II molecules in humans include HLA-DM, HLA-DO, HLA-DP, HLA-DQ and HLA-DR and include H-2 I-A and H-2 I-E in mice. MHC II expression is more restricted to B cells, dendritic cells, macrophages, activated T cells and thymic epithelial cells and MHC II molecules present peptides to CD4 lymphocytes.

### Antibodies to Target HLA-E/Cancer Peptides

Current approaches and technologies used for targeting MHC/peptide complexes have several limitations including but not limited to: (1) monoclonal agents to MHC/peptide targets already in pre-clinical and clinical development are specific to classical MHC class I molecules, which have been shown to be down-regulated in many cancers, (2) classical MHC I molecules are highly polymorphic limiting the population coverage for these targeting agents (3) the majority of peptides previously identified using tumor cell lines and direct methods reveal peptides derived from the conventional antigen processing route; even though many tumor cells are known to have defects in APM components, (4) choosing the right antigen is difficult, (5) low copy number expression of previously reported classical MHC/peptide targets potentially creates a technical hurdle for developing effective therapeutics, (6) large bulky size of the conventional antibodies and TCR molecules hinder the identification of useful epitopes that are hidden and could be recognized by significantly smaller less bulky molecules such as single-domain binders which are also highly soluble and stable molecules for easier and more cost effective manufacturing, and (7) first generation anti-MHC/peptide agents target a single MHC/peptide complex making it easier for tumor escape to occur. Identifying an ideal target requires consideration of the peptide abundance, presentation, specificity for cancer cells versus healthy cells, and heterogeneity of expression on tumor cells.

Camelid single-domain antibodies are derived from camels, llamas and alpacas, and are composed of approximately 110 amino acids comprising one variable domain (VH) of a heavy-chain antibody, or of a common IgG. Camelid antibodies include V_{H}H or single domain antibodies that are small ˜12 KD and tend to bind with high affinity. Also, these antibodies have good solubility and stability properties and are readily humanized. Camelid derived single-domain antibodies are able to bind to hidden antigens that are not accessible to whole antibodies, for example to the active sites of enzymes. V_{H}H antibodies have a protruding or convex paratope in contrast to the more concave paratope often seen for conventional antibodies. The convex like nature of the V_{H}H antibodies from llamas and their small size yields useful binders able to recognize narrow grooves and deep pockets. The HLA-E peptide-binding pocket with peptide has small deep grooves in the pocket that V_{H}H antibodies be suitable for recognizing due to their small size and protruding paratope. Furthermore, the low molecular mass leads to a better permeability in tissues making these antibody molecules potentially better at penetrating tumors. Additionally, their small size makes them highly conducive as multispecific and multivalent molecules.

Disclosed herein, in certain embodiments, are compositions that target a complex comprising a non-classical HLA-I and a neoantigen, and methods of use thereof. In some instances, the compositions comprise antibodies. In some instances, the antibodies are scFvs from mice and human libraries. In some instances, the antibodies are single domain antibodies derived from immunized llamas.

Disclosed herein, in certain embodiments, are antibodies that selectively bind to a complex comprising a non-classical HLA-I and a peptide. In some instances, the antibody does not have a binding affinity to the non-classical HLA-I alone. In some instances, the antibody does not have a binding affinity to the peptide alone. In some instances, the antibody does not have a binding affinity to a complex comprising the non-classical HLA-I and a non-relevant peptide.

In some instances, the peptide is expressed by an antigen processing machinery (APM)-proficient cell. In some instances, the peptide is expressed by a TAP1/2-proficient cell. In some instances, the peptide is expressed by an antigen processing machinery (APM)-deficient cell. In some instances, the peptide is expressed by a TAP1/2-deficient cell.

In some instances, the peptide comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), SEQ ID NO: 31 (VMAPRTLVL), SEQ ID NO: 19 (YLLPRRGPRL), SEQ ID NO: 20 (AISPRTLNA), SEQ ID NO: 21 (SQAPLPCVL), SEQ ID NO: 15 (YLLEMLWRL), SEQ ID NO: 16 (YMLDLQPETT), SEQ ID NO: 22 (QMRPVSRVL), SEQ ID NO: 23 (ALALVRMLI), SEQ ID NO: 24 (SQQPYLQLQ), SEQ ID NO: 25 (AMAPIKTHL), SEQ ID NO: 26 (AMAPIKVRL), SEQ ID NO: 17 (YLLPAIVHI), SEQ ID NO: 27 (ILDQKINEV), SEQ ID NO: 28 (GVYDGEEHSV), SEQ ID NO: 29 (KVLEYVIKV), SEQ ID NO: 18 (SLLMWITQV), SEQ ID NO: 30 (YLEPGPVTV), SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the peptide comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), or SEQ ID NO: 31 (VMAPRTLVL).

In some instances, the peptide comprises, consisting essentially of, or consisting of a sequence according to SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the peptide comprises, consisting essentially of, or consisting of a sequence according to SEQ ID NO. 3 (VMAPRTLFL). In some instances, the peptide comprises, consisting essentially of, or consisting of a sequence according to SEQ ID NO. 13 (VMAPRTLIL). In some instances, the peptide comprises, consisting essentially of, or consisting of a sequence according to SEQ ID NO. 14 (VMPPRTLLL). In some instances, the peptide comprises, consisting essentially of, or consisting of a sequence according to SEQ ID NO. 31 (VMAPRTLVL). In some instances, the peptide comprises, consisting essentially of, or consisting of a sequence according to SEQ ID NO. 19 (YLLPRRGPRL).). In some instances, the peptide comprises, consisting essentially of, or consisting of a sequence according to SEQ ID NO. 20 (AISPRTLNA). In some instances, the peptide comprises, consisting essentially of, or consisting of a sequence according to SEQ ID NO. 21 (SQAPLPCVL). In some instances, the peptide comprises, consisting essentially of, or consisting of a sequence according to SEQ ID NO. 15 (YLLEMLWRL). In some instances, the peptide comprises, consisting essentially of, or consisting of a sequence according to SEQ ID NO. 16 (YMLDLQPETT). In some instances, the peptide comprises, consisting essentially of, or consisting of a sequence according to SEQ ID NO. 22 (QMRPVSRVL). In some instances, the peptide comprises, consisting essentially of, or consisting of a sequence according to SEQ ID NO. 23 (ALALVRMLI). In some instances, the peptide comprises, consisting essentially of, or consisting of a sequence according to SEQ ID NO. 24 (SQQPYLQLQ). In some instances, the peptide comprises, consisting essentially of, or consisting of a sequence according to SEQ ID NO. 25 (AMAPIKTHL). In some instances, the peptide comprises, consisting essentially of, or consisting of a sequence according to SEQ ID NO. 26 (AMAPIKVRL). In some instances, the peptide comprises, consisting essentially of, or consisting of a sequence according to SEQ ID NO. 17 (YLLPAIVHI). In some instances, the peptide comprises, consisting essentially of, or consisting of a sequence according to SEQ ID NO. 27 (ILDQKINEV). In some instances, the peptide comprises, consisting essentially of, or consisting of a sequence according to SEQ ID NO. 28 (GVYDGEEHSV). In some instances, the peptide comprises, consisting essentially of, or consisting of a sequence according to SEQ ID NO. 29 (KVLEYVIKV). In some instances, the peptide comprises, consisting essentially of, or consisting of a sequence according to SEQ ID NO. 18 (SLLMWITQV). In some instances, the peptide comprises, consisting essentially of, or consisting of a sequence according to SEQ ID NO. 30 (YLEPGPVTV). In some instances, the peptide comprises, consisting essentially of, or consisting of a sequence according to SEQ ID NO. 32 (SLLEKSLGL). In some instances, the peptide comprises, consisting essentially of, or consisting of a sequence according to SEQ ID NO. 22 (QMRPVSRVL). In some instances, the peptide comprises, consisting essentially of, or consisting of a sequence according to SEQ ID NO. 33 (WIAAVTIAA). In some instances, the peptide comprises, consisting essentially of, or consisting of a sequence according to SEQ ID NO. 34 (TSDMPGTTL). In some instances, the peptide comprises, consisting essentially of, or consisting of a sequence according to SEQ ID NO. 35 (MLALLTQVA). In some instances, the peptide comprises, consisting essentially of, or consisting of a sequence according to SEQ ID NO. 36 (QMFEGPLAL). In some instances, the peptide comprises, consisting essentially of, or consisting of a sequence according to SEQ ID NO. 37 (VLWDRTFSL). In some instances, the peptide comprises, consisting essentially of, or consisting of a sequence according to SEQ ID NO. 38 (TLFFQQNAL). In some instances, the peptide comprises, consisting essentially of, or consisting of a sequence according to SEQ ID NO. 1 (GLADKVYFL). In some instances, the peptide comprises, consisting essentially of, or consisting of a sequence according to SEQ ID NO. 2 (ILSPTVVSI).

In some instances, the non-classical HLA-I is HLA-E, HLA-F, HLA-G, or HLA-H. In some instances, the non-classical HLA-I is HLA-E. In some instances, the HLA-E is HLA-E*0101. In some instances, the HLA-E is HLA-E*0103.

In some instances, the antibody selectively binds to the complex comprising the HLA-E and the peptide. In some instances, the antibody selectively binds to the complex comprising the HLA-E*0101 and the peptide. In some instances, the antibody selectively binds to the complex comprising the HLA-E*0103 and the peptide. In some instances, the antibody selectively binds to the complex comprising the HLA-E*0101 and the peptide, and to the complex comprising the HLA-E*0103 and the peptide.

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), HLA-E and VMAPRTLVL (SEQ ID NO: 31), HLA-E and YLLPRRGPRL (SEQ ID NO: 19), the HLA-E and AISPRTLNA (SEQ ID NO: 20), the HLA-E and SQAPLPCVL (SEQ ID NO: 21), the HLA-E and YLLEMLWRL (SEQ ID NO: 15), the HLA-E and YMLDLQPETT (SEQ ID NO: 16), the HLA-E and QMRPVSRVL (SEQ ID NO: 22), the HLA-E and ALALVRMLI (SEQ ID NO: 23), the HLA-E and SQQPYLQLQ (SEQ ID NO: 24), the HLA-E and AMAPIKTHL (SEQ ID NO: 25), the HLA-E and

AMAPIKVRL (SEQ ID NO: 26), the HLA-E and YLLPAIVHI (SEQ ID NO: 17), the HLA-E and ILDQKINEV (SEQ ID NO: 27), the HLA-E and GVYDGEEHSV (SEQ ID NO: 28), the HLA-E and KVLEYVIKV (SEQ ID NO: 29), the HLA-E and SLLMWITQV (SEQ ID NO: 18), the HLA-E and YLEPGPVTV (SEQ ID NO: 30), the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), or HLA-E and VMAPRTLVL (SEQ ID NO: 31).

In some instances, the complex comprises the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO. 3). In some instances, the complex comprises the HLA-E and VMAPRTLIL (SEQ ID NO. 13). In some instances, the complex comprises the HLA-E and VMPPRTLLL (SEQ ID NO. 14). In some instances, the complex comprises the HLA-E and VMAPRTLVL (SEQ ID NO. 31). In some instances, the complex comprises the HLA-E and YLLPRRGPRL (SEQ ID NO. 19). In some instances, the complex comprises the HLA-E and AISPRTLNA (SEQ ID NO. 20). In some instances, the complex comprises the HLA-E and SQAPLPCVL (SEQ ID NO. 21). In some instances, the complex comprises the HLA-E and YLLEMLWRL (SEQ ID NO. 15). In some instances, the complex comprises the HLA-E and YMLDLQPETT (SEQ ID NO. 16). In some instances, the complex comprises the HLA-E and QMRPVSRVL (SEQ ID NO. 22). In some instances, the complex comprises the HLA-E and ALALVRMLI (SEQ ID NO. 23). In some instances, the complex comprises the HLA-E and SQQPYLQLQ (SEQ ID NO. 24). In some instances, the complex comprises the HLA-E and AMAPIKTHL (SEQ ID NO. 25). In some instances, the complex comprises the HLA-E and AMAPIKVRL (SEQ ID NO. 26). In some instances, the complex comprises the HLA-E and YLLPAIVHI (SEQ ID NO. 17). In some instances, the complex comprises the HLA-E and ILDQKINEV (SEQ ID NO. 27). In some instances, the complex comprises the HLA-E and GVYDGEEHSV (SEQ ID NO. 28). In some instances, the complex comprises the HLA-E and KVLEYVIKV (SEQ ID NO. 29). In some instances, the complex comprises the HLA-E and SLLMWITQV (SEQ ID NO. 18). In some instances, the complex comprises the HLA-E and YLEPGPVTV (SEQ ID NO. 30). In some instances, the complex comprises the HLA-E and SLLEKSLGL (SEQ ID NO. 32). In some instances, the complex comprises the HLA-E and QMRPVSRVL (SEQ ID NO. 22). In some instances, the complex comprises the HLA-E and WIAAVTIAA (SEQ ID NO. 33). In some instances, the complex comprises the HLA-E and TSDMPGTTL (SEQ ID NO. 34). In some instances, the complex comprises the HLA-E and MLALLTQVA (SEQ ID NO. 35). In some instances, the complex comprises the HLA-E and QMFEGPLAL (SEQ ID NO. 36). In some instances, the complex comprises the HLA-E and VLWDRTFSL (SEQ ID NO. 37). In some instances, the complex comprises the HLA-E and TLFFQQNAL (SEQ ID NO. 38). In some instances, the complex comprises the HLA-E and GLADKVYFL (SEQ ID NO. 1). In some instances, the complex comprises the HLA-E and ILSPTVVSI (SEQ ID NO. 2).

In some instances, the antibody is a murine antibody. In some instances, the antibody is a chimeric antibody. In some instances, the antibody is a camelid antibody. In some instances, the antibody is a humanized antibody. In some instances, the antibody is a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody.

In some instances, the antibody further comprises a conjugated therapeutic moiety. In some instances, the selective binding of the antibody to the complex comprising the non-classical HLA-I and the peptide induces an immune response. In some instances, the immune response comprises activation of T cells. In some instances, the T cell is a CD8+ T cell. In some instances, the immune response comprises activation of cytotoxic T cells (CTLs).

In some instances, the cell is a cancer cell. In some instances, the cancer cell is breast cancer cell. In some instances, the cancer cell is kidney cancer cell. In some instances, the cancer cell is lung cancer cell. In some instances, the cancer cell is ovarian cancer cell. In some instances, the cancer cell is colorectal cancer cell. In some instances, the cancer cell is a B-cell malignancy cancer cell.

### Methods of Treatment

Disclosed herein, in some embodiments, are methods of treating cancer in an individual in need thereof comprising administering to the individual an antibody that selectively binds to a complex comprising a non-classical HLA-I and a neoantigen. In some instances, the antibody does not have a binding affinity to the non-classical HLA-I alone. In some instances, the antibody does not have a binding affinity to the neoantigen alone. In some instances, the antibody does not have a binding affinity to a complex comprising the non-classical HLA-I and a non-relevant neoantigen.

In some instances, the neoantigen is expressed by an antigen processing machinery (APM)-proficient cell. In some instances, the neoantigen is expressed by a TAP1/2-proficient cell. In some instances, the neoantigen is expressed by an antigen processing machinery (APM)-deficient cell. In some instances, the neoantigen is expressed by a TAP1/2-deficient cell.

In some instances, the neoantigen comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), SEQ ID NO: 31 (VMAPRTLVL), SEQ ID NO: 19 (YLLPRRGPRL), SEQ ID NO: 20 (AISPRTLNA), SEQ ID NO: 21 (SQAPLPCVL), SEQ ID NO: 15 (YLLEMLWRL), SEQ ID NO: 16 (YMLDLQPETT), SEQ ID NO: 22 (QMRPVSRVL), SEQ ID NO: 23 (ALALVRMLI), SEQ ID NO: 24 (SQQPYLQLQ), SEQ ID NO: 25 (AMAPIKTHL), SEQ ID NO: 26 (AMAPIKVRL), SEQ ID NO: 17 (YLLPAIVHI), SEQ ID NO: 27 (ILDQKINEV), SEQ ID NO: 28 (GVYDGEEHSV), SEQ ID NO: 29 (KVLEYVIKV), SEQ ID NO: 18 (SLLMWITQV), SEQ ID NO: 30 (YLEPGPVTV), SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the neoantigen comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), or SEQ ID NO: 31 (VMAPRTLVL).

In some instances, the neoantigen comprises, consisting essentially of, or consisting of a sequence according to SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the neoantigen comprises, consisting essentially of, or consisting of a sequence according to SEQ ID NO. 3 (VMAPRTLFL). In some instances, the neoantigen comprises, consisting essentially of, or consisting of a sequence according to SEQ ID NO. 13 (VMAPRTLIL). In some instances, the neoantigen comprises, consisting essentially of, or consisting of a sequence according to SEQ ID NO. 14 (VMPPRTLLL). In some instances, the neoantigen comprises, consisting essentially of, or consisting of a sequence according to SEQ ID NO. 31 (VMAPRTLVL). In some instances, the neoantigen comprises, consisting essentially of, or consisting of a sequence according to SEQ ID NO. 19 (YLLPRRGPRL). In some instances, the neoantigen comprises, consisting essentially of, or consisting of a sequence according to SEQ ID NO. 20 (AISPRTLNA). In some instances, the neoantigen comprises, consisting essentially of, or consisting of a sequence according to SEQ ID NO. 21 (SQAPLPCVL). In some instances, the neoantigen comprises, consisting essentially of, or consisting of a sequence according to SEQ ID NO. 15 (YLLEMLWRL). In some instances, the neoantigen comprises, consisting essentially of, or consisting of a sequence according to SEQ ID NO. 16 (YMLDLQPETT). In some instances, the neoantigen comprises, consisting essentially of, or consisting of a sequence according to SEQ ID NO. 22 (QMRPVSRVL). In some instances, the neoantigen comprises, consisting essentially of, or consisting of a sequence according to SEQ ID NO. 23 (ALALVRMLI). In some instances, the neoantigen comprises, consisting essentially of, or consisting of a sequence according to SEQ ID NO. 24 (SQQPYLQLQ). In some instances, the neoantigen comprises, consisting essentially of, or consisting of a sequence according to SEQ ID NO. 25 (AMAPIKTHL). In some instances, the neoantigen comprises, consisting essentially of, or consisting of a sequence according to SEQ ID NO. 26 (AMAPIKVRL). In some instances, the neoantigen comprises, consisting essentially of, or consisting of a sequence according to SEQ ID NO. 17 (YLLPAIVHI). In some instances, the neoantigen comprises, consisting essentially of, or consisting of a sequence according to SEQ ID NO. 27 (ILDQKINEV). In some instances, the neoantigen comprises, consisting essentially of, or consisting of a sequence according to SEQ ID NO. 28 (GVYDGEEHSV). In some instances, the neoantigen comprises, consisting essentially of, or consisting of a sequence according to SEQ ID NO. 29 (KVLEYVIKV). In some instances, the neoantigen comprises, consisting essentially of, or consisting of a sequence according to SEQ ID NO. 18 (SLLMWITQV). In some instances, the neoantigen comprises, consisting essentially of, or consisting of a sequence according to SEQ ID NO. 30 (YLEPGPVTV). In some instances, the neoantigen comprises, consisting essentially of, or consisting of a sequence according to SEQ ID NO. 32 (SLLEKSLGL). In some instances, the neoantigen comprises, consisting essentially of, or consisting of a sequence according to SEQ ID NO. 22 (QMRPVSRVL). In some instances, the neoantigen comprises, consisting essentially of, or consisting of a sequence according to SEQ ID NO. 33 (WIAAVTIAA). In some instances, the neoantigen comprises, consisting essentially of, or consisting of a sequence according to SEQ ID NO. 34 (TSDMPGTTL). In some instances, the neoantigen comprises, consisting essentially of, or consisting of a sequence according to SEQ ID NO. 35 (MLALLTQVA). In some instances, the neoantigen comprises, consisting essentially of, or consisting of a sequence according to SEQ ID NO. 36 (QMFEGPLAL). In some instances, the neoantigen comprises, consisting essentially of, or consisting of a sequence according to SEQ ID NO. 37 (VLWDRTFSL). In some instances, the neoantigen comprises, consisting essentially of, or consisting of a sequence according to SEQ ID NO. 38 (TLFFQQNAL). In some instances, the neoantigen comprises, consisting essentially of, or consisting of a sequence according to SEQ ID NO. 1 (GLADKVYFL). In some instances, the neoantigen comprises, consisting essentially of, or consisting of a sequence according to SEQ ID NO. 2 (ILSPTVVSI).

In some instances, the non-classical HLA-I is HLA-E, HLA-F, HLA-G, or HLA-H. In some instances, the non-classical HLA-I is HLA-E. In some instances, the HLA-E is HLA-E*0101. In some instances, the HLA-E is HLA-E*0103.

In some instances, the antibody selectively binds to the complex comprising the HLA-E and the neoantigen. In some instances, the antibody selectively binds to the complex comprising the HLA-E*0101 and the neoantigen. In some instances, the antibody selectively binds to the complex comprising the HLA-E*0103 and the neoantigen. In some instances, the antibody selectively binds to the complex comprising the HLA-E*0101 and the neoantigen, and to the complex of the HLA-E*0103 and the neoantigen.

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), HLA-E and VMAPRTLVL (SEQ ID NO: 31), HLA-E and YLLPRRGPRL (SEQ ID NO: 19), the HLA-E and AISPRTLNA (SEQ ID NO: 20), the HLA-E and SQAPLPCVL (SEQ ID NO: 21), the HLA-E and YLLEMLWRL (SEQ ID NO: 15), the HLA-E and YMLDLQPETT (SEQ ID NO: 16), the HLA-E and QMRPVSRVL (SEQ ID NO: 22), the HLA-E and ALALVRMLI (SEQ ID NO: 23), the HLA-E and SQQPYLQLQ (SEQ ID NO: 24), the HLA-E and AMAPIKTHL (SEQ ID NO: 25), the HLA-E and AMAPIKVRL (SEQ ID NO: 26), the HLA-E and YLLPAIVHI (SEQ ID NO: 17), the HLA-E and ILDQKINEV (SEQ ID NO: 27), the HLA-E and GVYDGEEHSV (SEQ ID NO: 28), the HLA-E and KVLEYVIKV (SEQ ID NO: 29), the HLA-E and SLLMWITQV (SEQ ID NO: 18), the HLA-E and YLEPGPVTV (SEQ ID NO: 30), the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), or HLA-E and VMAPRTLVL (SEQ ID NO: 31). In some instances, the complex is the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO. 3). In some instances, the complex comprises the HLA-E and VMAPRTLIL (SEQ ID NO. 13). In some instances, the complex comprises the HLA-E and VMPPRTLLL (SEQ ID NO. 14). In some instances, the complex comprises the HLA-E and VMAPRTLVL (SEQ ID NO. 31). In some instances, the complex comprises the HLA-E and YLLPRRGPRL (SEQ ID NO. 19). In some instances, the complex comprises the HLA-E and AISPRTLNA (SEQ ID NO. 20). In some instances, the complex comprises the HLA-E and SQAPLPCVL (SEQ ID NO. 21). In some instances, the complex comprises the HLA-E and YLLEMLWRL (SEQ ID NO. 15). In some instances, the complex comprises the HLA-E and YMLDLQPETT (SEQ ID NO. 16). In some instances, the complex comprises the HLA-E and QMRPVSRVL (SEQ ID NO. 22). In some instances, the complex comprises the HLA-E and ALALVRMLI (SEQ ID NO. 23). In some instances, the complex comprises the HLA-E and SQQPYLQLQ (SEQ ID NO. 24). In some instances, the complex comprises the HLA-E and AMAPIKTHL (SEQ ID NO. 25). In some instances, the complex comprises the HLA-E and AMAPIKVRL (SEQ ID NO. 26). In some instances, the complex comprises the HLA-E and YLLPAIVHI (SEQ ID NO. 17). In some instances, the complex comprises the HLA-E and ILDQKINEV (SEQ ID NO. 27). In some instances, the complex comprises the HLA-E and GVYDGEEHSV (SEQ ID NO. 28). In some instances, the complex comprises the HLA-E and KVLEYVIKV (SEQ ID NO. 29). In some instances, the complex comprises the HLA-E and SLLMWITQV (SEQ ID NO. 18). In some instances, the complex comprises the HLA-E and YLEPGPVTV (SEQ ID NO. 30). In some instances, the complex comprises the HLA-E and SLLEKSLGL (SEQ ID NO. 32). In some instances, the complex comprises the HLA-E and QMRPVSRVL (SEQ ID NO. 22). In some instances, the complex comprises the HLA-E and WIAAVTIAA (SEQ ID NO. 33). In some instances, the complex comprises the HLA-E and TSDMPGTTL (SEQ ID NO. 34). In some instances, the complex comprises the HLA-E and MLALLTQVA (SEQ ID NO. 35). In some instances, the complex comprises the HLA-E and QMFEGPLAL (SEQ ID NO. 36). In some instances, the complex comprises the HLA-E and VLWDRTFSL (SEQ ID NO. 37). In some instances, the complex comprises the HLA-E and TLFFQQNAL (SEQ ID NO. 38). In some instances, the complex comprises the HLA-E and GLADKVYFL (SEQ ID NO. 1). In some instances, the complex comprises the HLA-E and ILSPTVVSI (SEQ ID NO. 2).

In some instances, the antibody is a murine antibody. In some instances, the antibody is a chimeric antibody. In some instances, the antibody is a camelid antibody. In some instances, the antibody is a humanized antibody. In some instances, the antibody is a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody.

In some instances, the antibody further comprises a conjugated therapeutic moiety. In some instances, the selective binding of the antibody to the complex comprising the non-classical HLA-I and the neoantigen induces an immune response. In some instances, the immune response comprises activation of T cells. In some instances, the T cell is a CD8+ T cell. In some instances, the immune response comprises activation of cytotoxic T cells (CTLs).

In some instances, the antibody is administered continuously for 1, 2, 3, 4, 5, 6, 7, 8, 9, 10, 14, 15, 28, 30 or more days. In some instances, the antibody is administered at predetermined time intervals for 1, 2, 3, 4, 5, 6, 7, 8, 9, 10, 14, 15, 28, 30 or more days. In some instances, the antibody is administered is administered intermittently for 1, 2, 3, 4, 5, 6, 7, 8, 9, 10, 14, 15, 28, 30 or more days. In some instances, the antibody is administered in 1 dose, 2 doses, 3 doses, 4 doses, 5 doses, 6 doses or more. In some instances, the antibody is administered at a therapeutically effective amount.

In some instances, the cancer is breast cancer. In some instances, the cancer is kidney cancer. In some instances, the cancer is lung cancer. In some instances, the cancer is ovarian cancer. In some instances, the cancer is colorectal cancer. In some instances, the cancer is a B-cell malignancy.

In some embodiments, disclosed herein, are methods of treating cancer in an individual in need thereof, comprising administering to the individual an antibody that selectively binds to a complex comprising an HLA-E and a neoantigen. In some instances, the antibody does not have a binding affinity to the non-classical HLA-I alone. In some instances, the antibody does not have a binding affinity to the neoantigen alone. In some instances, the antibody does not have a binding affinity to a complex comprising the non-classical HLA-I and a non-relevant neoantigen.

In some instances, the neoantigen is expressed by an antigen processing machinery (APM)-proficient cell. In some instances, the neoantigen is expressed by a TAP1/2-proficient cell. In some instances, the neoantigen is expressed by an antigen processing machinery (APM)-deficient cell. In some instances, the neoantigen is expressed by a TAP1/2-deficient cell.

In some instances, the neoantigen comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), SEQ ID NO: 31 (VMAPRTLVL), SEQ ID NO: 19 (YLLPRRGPRL), SEQ ID NO: 20 (AISPRTLNA), SEQ ID NO: 21 (SQAPLPCVL), SEQ ID NO: 15 (YLLEMLWRL), SEQ ID NO: 16 (YMLDLQPETT), SEQ ID NO: 22 (QMRPVSRVL), SEQ ID NO: 23 (ALALVRMLI), SEQ ID NO: 24 (SQQPYLQLQ), SEQ ID NO: 25 (AMAPIKTHL), SEQ ID NO: 26 (AMAPIKVRL), SEQ ID NO: 17 (YLLPAIVHI), SEQ ID NO: 27 (ILDQKINEV), SEQ ID NO: 28 (GVYDGEEHSV), SEQ ID NO: 29 (KVLEYVIKV), SEQ ID NO: 18 (SLLMWITQV), SEQ ID NO: 30 (YLEPGPVTV), SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the neoantigen comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), or SEQ ID NO: 31 (VMAPRTLVL).

In some instances, the neoantigen comprises, consisting essentially of, or consisting of a sequence according to SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the neoantigen comprises, consisting essentially of, or consisting of a sequence according to SEQ ID NO. 3 (VMAPRTLFL). In some instances, the neoantigen comprises, consisting essentially of, or consisting of a sequence according to SEQ ID NO. 13 (VMAPRTLIL). In some instances, the neoantigen comprises, consisting essentially of, or consisting of a sequence according to SEQ ID NO. 14 (VMPPRTLLL). In some instances, the neoantigen comprises, consisting essentially of, or consisting of a sequence according to SEQ ID NO. 31 (VMAPRTLVL). In some instances, the neoantigen comprises, consisting essentially of, or consisting of a sequence according to SEQ ID NO. 19 (YLLPRRGPRL). In some instances, the neoantigen comprises, consisting essentially of, or consisting of a sequence according to SEQ ID NO. 20 (AISPRTLNA). In some instances, the neoantigen comprises, consisting essentially of, or consisting of a sequence according to SEQ ID NO. 21 (SQAPLPCVL). In some instances, the neoantigen comprises, consisting essentially of, or consisting of a sequence according to SEQ ID NO. 15 (YLLEMLWRL). In some instances, the neoantigen comprises, consisting essentially of, or consisting of a sequence according to SEQ ID NO. 16 (YMLDLQPETT). In some instances, the neoantigen comprises, consisting essentially of, or consisting of a sequence according to SEQ ID NO. 22 (QMRPVSRVL). In some instances, the neoantigen comprises, consisting essentially of, or consisting of a sequence according to SEQ ID NO. 23 (ALALVRMLI). In some instances, the neoantigen comprises, consisting essentially of, or consisting of a sequence according to SEQ ID NO. 24 (SQQPYLQLQ). In some instances, the neoantigen comprises, consisting essentially of, or consisting of a sequence according to SEQ ID NO. 25 (AMAPIKTHL). In some instances, the neoantigen comprises, consisting essentially of, or consisting of a sequence according to SEQ ID NO. 26 (AMAPIKVRL). In some instances, the neoantigen comprises, consisting essentially of, or consisting of a sequence according to SEQ ID NO. 17 (YLLPAIVHI). In some instances, the neoantigen comprises, consisting essentially of, or consisting of a sequence according to SEQ ID NO. 27 (ILDQKINEV). In some instances, the neoantigen comprises, consisting essentially of, or consisting of a sequence according to SEQ ID NO. 28 (GVYDGEEHSV). In some instances, the neoantigen comprises, consisting essentially of, or consisting of a sequence according to SEQ ID NO. 29 (KVLEYVIKV). In some instances, the neoantigen comprises, consisting essentially of, or consisting of a sequence according to SEQ ID NO. 18 (SLLMWITQV). In some instances, the neoantigen comprises, consisting essentially of, or consisting of a sequence according to SEQ ID NO. 30 (YLEPGPVTV). In some instances, the neoantigen comprises, consisting essentially of, or consisting of a sequence according to SEQ ID NO. 32 (SLLEKSLGL). In some instances, the neoantigen comprises, consisting essentially of, or consisting of a sequence according to SEQ ID NO. 22 (QMRPVSRVL). In some instances, the neoantigen comprises, consisting essentially of, or consisting of a sequence according to SEQ ID NO. 33 (WIAAVTIAA). In some instances, the neoantigen comprises, consisting essentially of, or consisting of a sequence according to SEQ ID NO. 34 (TSDMPGTTL). In some instances, the neoantigen comprises, consisting essentially of, or consisting of a sequence according to SEQ ID NO. 35 (MLALLTQVA). In some instances, the neoantigen comprises, consisting essentially of, or consisting of a sequence according to SEQ ID NO. 36 (QMFEGPLAL). In some instances, the neoantigen comprises, consisting essentially of, or consisting of a sequence according to SEQ ID NO. 37 (VLWDRTFSL). In some instances, the neoantigen comprises, consisting essentially of, or consisting of a sequence according to SEQ ID NO. 38 (TLFFQQNAL). In some instances, the neoantigen comprises, consisting essentially of, or consisting of a sequence according to SEQ ID NO. 1 (GLADKVYFL). In some instances, the neoantigen comprises, consisting essentially of, or consisting of a sequence according to SEQ ID NO. 2 (ILSPTVVSI).

In some instances, the HLA-E is HLA-E*0101. In some instances, the HLA-E is HLA-E*0103. In some instances, the antibody selectively binds to the complex comprising the HLA-E and the neoantigen. In some instances, the antibody selectively binds to the complex comprising the HLA-E*0101 and the neoantigen. In some instances, the antibody selectively binds to the complex comprising the HLA-E*0103 and the neoantigen. In some instances, the antibody selectively binds to the complex comprising the HLA-E*0101 and the neoantigen, and to the complex comprising the HLA-E*0103 and the neoantigen.

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), HLA-E and VMAPRTLVL (SEQ ID NO: 31), HLA-E and YLLPRRGPRL (SEQ ID NO: 19), the HLA-E and AISPRTLNA (SEQ ID NO: 20), the HLA-E and SQAPLPCVL (SEQ ID NO: 21), the HLA-E and YLLEMLWRL (SEQ ID NO: 15), the HLA-E and YMLDLQPETT (SEQ ID NO: 16), the HLA-E and QMRPVSRVL (SEQ ID NO: 22), the HLA-E and ALALVRMLI (SEQ ID NO: 23), the HLA-E and SQQPYLQLQ (SEQ ID NO: 24), the HLA-E and AMAPIKTHL (SEQ ID NO: 25), the HLA-E and AMAPIKVRL (SEQ ID NO: 26), the HLA-E and YLLPAIVHI (SEQ ID NO: 17), the HLA-E and ILDQKINEV (SEQ ID NO: 27), the HLA-E and GVYDGEEHSV (SEQ ID NO: 28), the HLA-E and KVLEYVIKV (SEQ ID NO: 29), the HLA-E and SLLMWITQV (SEQ ID NO: 18), the HLA-E and YLEPGPVTV (SEQ ID NO: 30), the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), or HLA-E and VMAPRTLVL (SEQ ID NO: 31).

In some instances, the complex comprises the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO. 3). In some instances, the complex comprises the HLA-E and VMAPRTLIL (SEQ ID NO. 13). In some instances, the complex comprises the HLA-E and VMPPRTLLL (SEQ ID NO. 14). In some instances, the complex comprises the HLA-E and VMAPRTLVL (SEQ ID NO. 31). In some instances, the complex comprises the HLA-E and YLLPRRGPRL (SEQ ID NO. 19). In some instances, the complex comprises the HLA-E and AISPRTLNA (SEQ ID NO. 20). In some instances, the complex comprises the HLA-E and SQAPLPCVL (SEQ ID NO. 21). In some instances, the complex comprises the HLA-E and YLLEMLWRL (SEQ ID NO. 15). In some instances, the complex comprises the HLA-E and YMLDLQPETT (SEQ ID NO. 16). In some instances, the complex comprises the HLA-E and QMRPVSRVL (SEQ ID NO. 22). In some instances, the complex comprises the HLA-E and ALALVRMLI (SEQ ID NO. 23). In some instances, the complex comprises the HLA-E and SQQPYLQLQ (SEQ ID NO. 24). In some instances, the complex comprises the HLA-E and AMAPIKTHL (SEQ ID NO. 25). In some instances, the complex comprises the HLA-E and AMAPIKVRL (SEQ ID NO. 26). In some instances, the complex comprises the HLA-E and YLLPAIVHI (SEQ ID NO. 17). In some instances, the complex comprises the HLA-E and ILDQKINEV (SEQ ID NO. 27). In some instances, the complex comprises the HLA-E and GVYDGEEHSV (SEQ ID NO. 28). In some instances, the complex comprises the HLA-E and KVLEYVIKV (SEQ ID NO. 29). In some instances, the complex comprises the HLA-E and SLLMWITQV (SEQ ID NO. 18). In some instances, the complex comprises the HLA-E and YLEPGPVTV (SEQ ID NO. 30). In some instances, the complex comprises the HLA-E and SLLEKSLGL (SEQ ID NO. 32). In some instances, the complex comprises the HLA-E and QMRPVSRVL (SEQ ID NO. 22). In some instances, the complex comprises the HLA-E and WIAAVTIAA (SEQ ID NO. 33). In some instances, the complex comprises the HLA-E and TSDMPGTTL (SEQ ID NO. 34). In some instances, the complex comprises the HLA-E and MLALLTQVA (SEQ ID NO. 35). In some instances, the complex comprises the HLA-E and QMFEGPLAL (SEQ ID NO. 36). In some instances, the complex comprises the HLA-E and VLWDRTFSL (SEQ ID NO. 37). In some instances, the complex comprises the HLA-E and TLFFQQNAL (SEQ ID NO. 38). In some instances, the complex comprises the HLA-E and GLADKVYFL (SEQ ID NO. 1). In some instances, the complex comprises the HLA-E and ILSPTVVSI (SEQ ID NO. 2).

In some instances, the antibody is a murine antibody. In some instances, the antibody is a chimeric antibody. In some instances, the antibody is a camelid antibody. In some instances, the antibody is a humanized antibody. In some instances, the antibody is a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody.

In some instances, the antibody further comprises a conjugated therapeutic moiety. In some instances, the selective binding of the antibody to the complex comprising the non-classical HLA-I and the neoantigen induces an immune response. In some instances, the immune response comprises activation of T cells. In some instances, the T cell is a CD8+ T cell. In some instances, the immune response comprises activation of cytotoxic T cells (CTLs).

In some instances, the antibody is administered continuously for 1, 2, 3, 4, 5, 6, 7, 8, 9, 10, 14, 15, 28, 30 or more days. In some instances, the antibody is administered at predetermined time intervals for 1, 2, 3, 4, 5, 6, 7, 8, 9, 10, 14, 15, 28, 30 or more days. In some instances, the antibody is administered is administered intermittently for 1, 2, 3, 4, 5, 6, 7, 8, 9, 10, 14, 15, 28, 30 or more days. In some instances, the antibody is administered in 1 dose, 2 doses, 3 doses, 4 doses, 5 doses, 6 doses or more. In some instances, the antibody is administered at a therapeutically effective amount.

In some instances, the cancer is breast cancer. In some instances, the cancer is kidney cancer. In some instances, the cancer is lung cancer. In some instances, the cancer is ovarian cancer. In some instances, the cancer is colorectal cancer. In some instances, the cancer is a B-cell malignancy.

### Pharmaceutical Compositions and Formulations

Also disclosed herein are pharmaceutical compositions comprising antibodies disclosed herein that selectively bind to a complex comprising a non-classical HLA-I and a peptide; and a pharmaceutically acceptable carrier or excipient.

In some embodiments, excipients for use with the compositions disclosed herein include maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In some embodiments, the compositions further comprise an additional therapeutic agent. In some embodiments, the therapeutic agent is a chemotherapeutic agent. In some embodiments, the chemotherapeutic agents include, among others, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In some embodiments, the antibody and the therapeutic agent are in the same formulation. In some embodiments, the antibody and the therapeutic agent are in different formulation. In some embodiments, antibody described herein is used prior to the administration of the other therapeutic agent. In some embodiments, antibody described herein is used concurrently with the administration of the other therapeutic agent. In some embodiments, antibody described herein is used subsequent to the administration of the other therapeutic agent.

Pharmaceutical formulations, in some embodiments, are made to be compatible with a particular local, regional or systemic administration or delivery route. Thus, pharmaceutical formulations include carriers, diluents, or excipients suitable for administration by particular routes. Specific non-limiting examples of routes of administration for compositions herein are parenteral, e.g., intravenous, intra-arterial, intradermal, intramuscular, subcutaneous, intra-pleural, transdermal (topical), transmucosal, intra-cranial, intra-spinal, intra-ocular, rectal, oral (alimentary), mucosal administration, and any other formulation suitable for the treatment method or administration protocol.

In some embodiments, solutions or suspensions used for parenteral application include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. In some embodiments, pH is adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.), or phosphate buffered saline (PBS). In some embodiments, the carrier is a solvent or dispersion medium containing, for example, water, ethanol, polyol (for example, glycerol, propylene glycol, and liquid polyetheylene glycol, and the like), or suitable mixtures thereof. Fluidity is maintained, in some embodiments, for example, by the use of a coating such as lecithin, by the maintenance of the required particle size in the case of dispersion, and by the use of surfactants. Antibacterial and antifungal agents include, for example, parabens, chlorobutanol, phenol, ascorbic acid, and thimerosal. Isotonic agents, for example, sugars; polyalcohols such as mannitol or sorbitol; or sodium chloride, in some embodiments, are included in the composition. In some cases, also included is an agent which delays absorption, in some embodiments, for example, aluminum monostearate or gelatin prolongs absorption of injectable compositions.

In some embodiments, sterile injectable formulations are prepared by incorporating the active composition in the required amount in an appropriate solvent with one or a combination of above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. In some embodiments, transmucosal administration is accomplished through the use of nasal sprays, inhalation devices (e.g., aspirators) or suppositories. For transdermal administration, the active compounds are formulated into ointments, salves, gels, creams or patches.

In some embodiments, the pharmaceutical formulations are prepared with carriers that protect against rapid elimination from the body, such as a controlled release formulation or a time delay material such as glyceryl monostearate or glyceryl stearate. The formulations, in some embodiments, are also delivered using articles of manufacture such as implants and microencapsulated delivery systems to achieve local, regional or systemic delivery or controlled or sustained release.

### Therapeutic Regimens for a Pharmaceutical Composition

In some embodiments, a pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, in some instances, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular composition, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the composition used, the disease or condition to be treated, the mode of administration, the requirements of the individual subject, the severity of the disease or condition being treated, and the judgment of the practitioner. In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and EMI Compositions exhibiting high therapeutic indices are preferred. The data obtained from cell culture assays and animal studies are used in formulating a range of dosage for use in human. The dosage of such composition lies preferably within a range of circulating concentrations that include the ED50 with minimal toxicity. The dosage varies within this range depending upon the dosage form employed and the route of administration utilized.

### Methods of Producing Antibodies

Disclosed herein, in some embodiments, are compositions and methods of producing such compositions that target a complex comprising a non-classical HLA-I and a neoantigen. In some instances, the compositions comprise an antibody. In some instances, the antibody is a camelid antibody.

In some embodiments, disclosed herein, are methods of producing a camelid antibody that selectively binds to a complex comprising a non-classical HLA-I and a peptide, the method comprising: (a) administering an effective amount of an immunogen to a camelid for eliciting an immune response, wherein the immunogen comprises a recombinantly expressed complex of a non-classical HLA-I and a peptide; (b) constructing an antibody library; (c) assaying the antibody library to select the antibody; and (d) isolating the antibody. In some instances, the antibody does not have a binding affinity to the non-classical HLA-I alone. In some instances, the antibody does not have a binding affinity to the peptide alone. In some instances, the antibody does not have a binding affinity to a complex comprising the non-classical HLA-I and a non-relevant peptide.

In some instances, the peptide is expressed by an antigen processing machinery (APM)-proficient cell. In some instances, the peptide is expressed by a TAP1/2-proficient cell. In some instances, the peptide is expressed by an antigen processing machinery (APM)-deficient cell. In some instances, the peptide is expressed by a TAP1/2-deficient cell.

In some instances, the peptide comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), SEQ ID NO: 31 (VMAPRTLVL), SEQ ID NO: 19 (YLLPRRGPRL), SEQ ID NO: 20 (AISPRTLNA), SEQ ID NO: 21 (SQAPLPCVL), SEQ ID NO: 15 (YLLEMLWRL), SEQ ID NO: 16 (YMLDLQPETT), SEQ ID NO: 22 (QMRPVSRVL), SEQ ID NO: 23 (ALALVRMLI), SEQ ID NO: 24 (SQQPYLQLQ), SEQ ID NO: 25 (AMAPIKTHL), SEQ ID NO: 26 (AMAPIKVRL), SEQ ID NO: 17 (YLLPAIVHI), SEQ ID NO: 27 (ILDQKINEV), SEQ ID NO: 28 (GVYDGEEHSV), SEQ ID NO: 29 (KVLEYVIKV), SEQ ID NO: 18 (SLLMWITQV), SEQ ID NO: 30 (YLEPGPVTV), SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the peptide comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), or SEQ ID NO: 31 (VMAPRTLVL).

In some instances, the peptide comprises, consisting essentially of, or consisting of a sequence according to SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

In some instances, the peptide comprises, consisting essentially of, or consisting of a sequence according to SEQ ID NO. 3 (VMAPRTLFL). In some instances, the peptide comprises, consisting essentially of, or consisting of a sequence according to SEQ ID NO. 13 (VMAPRTLIL). In some instances, the peptide comprises, consisting essentially of, or consisting of a sequence according to SEQ ID NO. 14 (VMPPRTLLL). In some instances, the peptide comprises, consisting essentially of, or consisting of a sequence according to SEQ ID NO. 31 (VMAPRTLVL). In some instances, the peptide comprises, consisting essentially of, or consisting of a sequence according to SEQ ID NO. 19 (YLLPRRGPRL). In some instances, the peptide comprises, consisting essentially of, or consisting of a sequence according to SEQ ID NO. 20 (AISPRTLNA). In some instances, the peptide comprises, consisting essentially of, or consisting of a sequence according to SEQ ID NO. 21 (SQAPLPCVL). In some instances, the peptide comprises, consisting essentially of, or consisting of a sequence according to SEQ ID NO. 15 (YLLEMLWRL). In some instances, the peptide comprises, consisting essentially of, or consisting of a sequence according to SEQ ID NO. 16 (YMLDLQPETT). In some instances, the peptide comprises, consisting essentially of, or consisting of a sequence according to SEQ ID NO. 22 (QMRPVSRVL). In some instances, the peptide comprises, consisting essentially of, or consisting of a sequence according to SEQ ID NO. 23 (ALALVRMLI). In some instances, the peptide comprises, consisting essentially of, or consisting of a sequence according to SEQ ID NO. 24 (SQQPYLQLQ). In some instances, the peptide comprises, consisting essentially of, or consisting of a sequence according to SEQ ID NO. 25 (AMAPIKTHL). In some instances, the peptide comprises, consisting essentially of, or consisting of a sequence according to SEQ ID NO. 26 (AMAPIKVRL). In some instances, the peptide comprises, consisting essentially of, or consisting of a sequence according to SEQ ID NO. 17 (YLLPAIVHI). In some instances, the peptide comprises, consisting essentially of, or consisting of a sequence according to SEQ ID NO. 27 (ILDQKINEV). In some instances, the peptide comprises, consisting essentially of, or consisting of a sequence according to SEQ ID NO. 28 (GVYDGEEHSV). In some instances, the peptide comprises, consisting essentially of, or consisting of a sequence according to SEQ ID NO. 29 (KVLEYVIKV). In some instances, the peptide comprises, consisting essentially of, or consisting of a sequence according to SEQ ID NO. 18 (SLLMWITQV). In some instances, the peptide comprises, consisting essentially of, or consisting of a sequence according to SEQ ID NO. 30 (YLEPGPVTV). In some instances, the peptide comprises, consisting essentially of, or consisting of a sequence according to SEQ ID NO. 32 (SLLEKSLGL). In some instances, the peptide comprises, consisting essentially of, or consisting of a sequence according to SEQ ID NO. 22 (QMRPVSRVL). In some instances, the peptide comprises, consisting essentially of, or consisting of a sequence according to SEQ ID NO. 33 (WIAAVTIAA). In some instances, the peptide comprises, consisting essentially of, or consisting of a sequence according to SEQ ID NO. 34 (TSDMPGTTL). In some instances, the peptide comprises, consisting essentially of, or consisting of a sequence according to SEQ ID NO. 35 (MLALLTQVA). In some instances, the peptide comprises, consisting essentially of, or consisting of a sequence according to SEQ ID NO. 36 (QMFEGPLAL). In some instances, the peptide comprises, consisting essentially of, or consisting of a sequence according to SEQ ID NO. 37 (VLWDRTFSL). In some instances, the peptide comprises, consisting essentially of, or consisting of a sequence according to SEQ ID NO. 38 (TLFFQQNAL). In some instances, the peptide comprises, consisting essentially of, or consisting of a sequence according to SEQ ID NO. 1 (GLADKVYFL). In some instances, the peptide comprises, consisting essentially of, or consisting of a sequence according to SEQ ID NO. 2 (ILSPTVVSI).

In some instances, the non-classical HLA-I is HLA-E, HLA-F, HLA-G, or HLA-H. In some instances, the non-classical HLA-I is HLA- E. In some instances, the HLA-E is HLA-E*0101. In some instances, the HLA-E is HLA-E*0103.

In some instances, the antibody selectively binds to the complex comprising the HLA-E and the peptide. In some instances, the antibody selectively binds to the complex comprising the HLA-E*0101 and the peptide. In some instances, the antibody selectively binds to the complex comprising the HLA-E*0103 and the peptide. In some instances, the antibody selectively binds to the complex comprising the HLA-E*0101 and the peptide, and to the complex of the HLA-E*0103 and the peptide.

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), HLA-E and VMAPRTLVL (SEQ ID NO: 31), HLA-E and YLLPRRGPRL (SEQ ID NO: 19), the HLA-E and AISPRTLNA (SEQ ID NO: 20), the HLA-E and SQAPLPCVL (SEQ ID NO: 21), the HLA-E and YLLEMLWRL (SEQ ID NO: 15), the HLA-E and YMLDLQPETT (SEQ ID NO: 16), the HLA-E and QMRPVSRVL (SEQ ID NO: 22), the HLA-E and ALALVRMLI (SEQ ID NO: 23), the HLA-E and SQQPYLQLQ (SEQ ID NO: 24), the HLA-E and AMAPIKTHL (SEQ ID NO: 25), the HLA-E and AMAPIKVRL (SEQ ID NO: 26), the HLA-E and YLLPAIVHI (SEQ ID NO: 17), the HLA-E and ILDQKINEV (SEQ ID NO: 27), the HLA-E and GVYDGEEHSV (SEQ ID NO: 28), the HLA-E and KVLEYVIKV (SEQ ID NO: 29), the HLA-E and SLLMWITQV (SEQ ID NO: 18), the HLA-E and YLEPGPVTV (SEQ ID NO: 30), the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), or HLA-E and VMAPRTLVL (SEQ ID NO: 31).

In some instances, the complex comprises the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

In some instances, the complex comprises the HLA-E and VMAPRTLFL (SEQ ID NO. 3). In some instances, the complex comprises the HLA-E and VMAPRTLIL (SEQ ID NO. 13). In some instances, the complex comprises the HLA-E and VMPPRTLLL (SEQ ID NO. 14). In some instances, the complex comprises the HLA-E and VMAPRTLVL (SEQ ID NO. 31). In some instances, the complex comprises the HLA-E and YLLPRRGPRL (SEQ ID NO. 19). In some instances, the complex comprises the HLA-E and AISPRTLNA (SEQ ID NO. 20). In some instances, the complex comprises the HLA-E and SQAPLPCVL (SEQ ID NO. 21). In some instances, the complex comprises the HLA-E and YLLEMLWRL (SEQ ID NO. 15). In some instances, the complex comprises the HLA-E and YMLDLQPETT (SEQ ID NO. 16). In some instances, the complex comprises the HLA-E and QMRPVSRVL (SEQ ID NO. 22). In some instances, the complex comprises the HLA-E and ALALVRMLI (SEQ ID NO. 23). In some instances, the complex comprises the HLA-E and SQQPYLQLQ (SEQ ID NO. 24). In some instances, the complex comprises the HLA-E and AMAPIKTHL (SEQ ID NO. 25). In some instances, the complex comprises the HLA-E and AMAPIKVRL (SEQ ID NO. 26). In some instances, the complex comprises the HLA-E and YLLPAIVHI (SEQ ID NO. 17). In some instances, the complex comprises the HLA-E and ILDQKINEV (SEQ ID NO. 27). In some instances, the complex comprises the HLA-E and GVYDGEEHSV (SEQ ID NO. 28). In some instances, the complex comprises the HLA-E and KVLEYVIKV (SEQ ID NO. 29). In some instances, the complex comprises the HLA-E and SLLMWITQV (SEQ ID NO. 18). In some instances, the complex comprises the HLA-E and YLEPGPVTV (SEQ ID NO. 30). In some instances, the complex comprises the HLA-E and SLLEKSLGL (SEQ ID NO. 32). In some instances, the complex comprises the HLA-E and QMRPVSRVL (SEQ ID NO. 22). In some instances, the complex comprises the HLA-E and WIAAVTIAA (SEQ ID NO. 33). In some instances, the complex comprises the HLA-E and TSDMPGTTL (SEQ ID NO. 34). In some instances, the complex comprises the HLA-E and MLALLTQVA (SEQ ID NO. 35). In some instances, the complex comprises the HLA-E and QMFEGPLAL (SEQ ID NO. 36). In some instances, the complex comprises the HLA-E and VLWDRTFSL (SEQ ID NO. 37). In some instances, the complex comprises the HLA-E and TLFFQQNAL (SEQ ID NO. 38). In some instances, the complex comprises the HLA-E and GLADKVYFL (SEQ ID NO. 1). In some instances, the complex comprises the HLA-E and ILSPTVVSI (SEQ ID NO. 2).

In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody.

In some instances, the antibody further comprises a conjugated therapeutic moiety. In some instances, the selective binding of the antibody to the complex comprising the non-classical HLA-I and the peptide induces an immune response. In some instances, the immune response comprises activation of T cells. In some instances, the T cell is a CD8+ T cell. In some instances, the immune response comprises activation of cytotoxic T cells (CTLs).

In some instances, the cell is a cancer cell. In some instances, the cancer cell is breast cancer cell. In some instances, the cancer cell is kidney cancer cell. In some instances, the cancer cell is lung cancer cell. In some instances, the cancer cell is ovarian cancer cell. In some instances, the cancer cell is colorectal cancer cell. In some instances, the cancer cell is a B-cell malignancy cancer cell.

In some instances, the immunogen is a monomer. In some instances, the immunogen is a tetramer. In some instances, the tetramer comprises avidin or derivatives thereof. In some instances, the immunogen is produced by recombinantly expressing an HLA-I heavy chain and a HLA-I light chain separately in E. coli, and then refolding the HLA-I heavy and light chains with peptide in vitro.

In some instances, the camelid is a llama. In some instances, the antibody library is a phage display library. In some instances, the antibody library is a bacteriophage display library. In some instances, the antibody library is a yeast display library. In some instances, the antibody library is a single domain antibody library.

### Method of Discovering Neoantigens/Neopeptides

Disclosed herein, in certain embodiments, are methods of discovering neoantigens/neopeptides. In some embodiments, the peptides presented by MHC I molecules are identified by indirect methods. In some instances, candidate peptides are tested for reactivity with peripheral blood mononuclear cells (PBMCs) isolated from patient blood. In some instances, the MHC/peptide complexes identified through indirect methods are those recognized by activated T cells. In some embodiments, peptides presented by MHC I molecules are identified by direct methods. In some instances, MHC/peptide complexes are identified by direct methods by identifying endogenously loaded peptides eluted from MHC molecules.

### Indirect Discovery

In some embodiments, indirect discovery of target classical MHC/peptide complexes use genomic, proteomic, or immunologic data to infer peptides that are presented by a particular MHC molecule during cancer, infection, or other disease states. In some instances, indirect approaches use genomic or proteomic techniques to identify proteins that are uniquely expressed or overexpressed in a disease state. Gene-centric approaches include, but are not limited to, real-time PCR, gene mutation analysis, differential display analysis, microarray experiments, and other genomic expression profiling methods. Protein-centric approaches include, but are not limited to, 2-D electrophoresis, mass spectrometric analysis of cell fractions, and other proteomic techniques to identify disease-associated proteins. After disease-associated genes and proteins are identified, algorithms or experimental peptide binding assays select candidate peptides. Expression profiling identifies candidate proteins from which representative peptides are synthesized and tested in vitro for binding to MHC.

In some instances, immune-centric approaches test candidate antigens for their presentation to CTLs. Cellular fractions from diseased cells are fed to dendritic cells (DC) for antigen processing and presentation. These DCs are then used to stimulate CTLs. In some instances, antigens that DC utilize to invoke a CTL response are considered candidates for further development.

In some instances, immune-centric assays are utilized to assess the immunologic potential of candidate MHC/peptide complexes. Bulk PBMC populations isolated from patients or healthy individuals (following in vitro stimulation) are assessed for CTL reactivity to pools of MHC/peptide complexes containing either synthetic overlapping peptide libraries or synthetic candidate peptides. Once the minimal epitope is identified, CTL clones specific to the MHC/peptide complex are generated from bulk PBMCs. Reactivity of T cell clones to diseased cells or cell lines is confirmed by target cell lysis via 51Cr release, interferon gamma release as detected by an enzyme-linked immunosorbent spot assay, or by intracellular cytokine staining.

### Traditional Direct Discovery

In some embodiments, direct approaches elute peptides directly from MHC/peptide complexes and identify the peptides specific to diseased cells. In some instances, direct discovery approaches identify peptides that are presented by the MHC molecules of cell lines. MHC/peptide complexes are first affinity purified from cell lysates. After MHC/peptide complexes are purified, direct discovery methods typically elute the peptides and identify disease-specific peptides by mass spectrometry.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an antibody, optionally with one or more additional therapeutic agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a composition provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing an antibody provided herein formulated in a compatible pharmaceutical carrier are also prepared, placed in an appropriate container, and labeled for treatment of an indicated condition.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Screening HLA-A2 Peptides for Binding to HLA-E*0101 and HLA-E*0103.

The strategy is to identify HLA-E peptide binders from a library of known peptides that bind HLA-A*0201. HLA-E*0103 peptide binding for YLLPAIVHI (SEQ ID NO: 17) from the protein RNA Helicase and SLLMWITQV (SEQ ID NO: 18) from the protein NY-ESO-1 has been shown and both peptides were used to successfully refold HLA-E*0103/peptide into stable monomer complexes. To identify additional peptide binders for HLA-E*0101 and HLA-E*0103, peptide binding studies and peptide/HLA-E refold assays are performed. Two lists of peptides were generated: List 1 consists of HLA-A*0201 binding peptides derived from the conventional processing route. List 2 shows peptide binders having the HLAA* 0201 binding motif derived from alternative processing routes. The peptides from this list were identified from a TAP-deficient cell line K562.HLA-E.B8 that was transduced with a retrovirus containing the UL49.5 gene of Bovine Herpes Virus-1. In total more than 200 peptides from each list have been screened.

### HLA-E Peptide Binding Assay

Binding affinity of peptides to HLA-E*0101 and HLA-E*0103 was determined in a cell-free competition-based refolding assay using recombinant HLA-E*0101 and HLA-E*0103. In brief, a fluorescence labeled natural ligand of HLA-E (VMAPC(FL)TLLL (SEQ ID NO: 39)) was used as standard and the eluted peptides were used as competitor. HLA-E*0101 and HLA-E*0103 was incubated in 96-well plates at RT (pH 7) for 24 h with 15 pmol beta2M and 100 fmol fluorescence labeled standard peptide, and a concentration series of the eluted peptides. The HLA-peptide complexes were separated from the free peptide through gel filtration and emission was measured at 528 nm. The percentage of peptide bound was calculated and the concentration of peptide yielding 50% inhibition (IC50) was deduced from the dose-response curve. The HLA-E reference peptide in this assay is the most optimal binder described, resulting in relatively high IC50 values. As a positive control, the leader peptide (VMAPRTLVL (SEQ ID NO: 31)) from HLA-A2 was used.

### HLA-E*0101 and HLA-E*0103 Peptide Refolding Assays

HLA-E*0101 and HLA-E*0103 extracellular domain and β2 m were produced as inclusion bodies in Escherichia coli and refolded with each peptide from both peptide lists. After refolding, the percent of properly folded complex was assessed on a Superdex 75 sizing column. Efficiency of refolded HLA-E monomers with peptides from both lists was compared to the control HLA-E monomer refolded with peptide VMAPRTLVL (SEQ ID NO: 31).

### Example 2

### In Silico Predictions of HLA-E Peptide Binders.

Housekeeping proteins (proteinatlas.org) having high turnover rates were surveyed using computational and informatics to predict peptide formation by alternative processing routes. Transcriptomics analysis of samples representing all major organs and tissues in the human body identified 8588 protein-coding genes responsible for the expression of housekeeping proteins (The HumanProteome). Web-based MHC epitope prediction tools and methods (SYFPEITHI (www.syfpeithi.de), The Immune Epitope Database and Analysis Resource (www.immunepitope.org), IMGT/HLA Sequence Database (www.ebi.ac.uk.imgt/hla/), Bimas (www-bimas.cit.nih.gov/molbio/hla_bind), and Prediction Algorithm for Proeasomal Cleavage (www.paproc.de) are used to predict HLA-A*0201 peptide binders. Peptides identified are rank-ordered based on predicted affinity for HLA-A*0201. The workflow is to assemble a list of “highest” affinity peptides for HLA-A*0201 and then to select, synthesize and screen peptides in HLA-E*0101 and HLA-E*0103 binding assays. Best binders to each HLA-E allele are assessed in refold reactions and refold efficiency is determined by comparing to control peptide VMAPRTLVL (SEQ ID NO: 31) used in HLA-E monomer refold reactions. HLA-A*0201 peptide binding motif is used to predict peptide binders to HLA-E.

### Example 3

De novo Discovery of HLA-E Neo-Peptides.

Existing tumor and endothelial cell lines (TAP-Dependent and -Independent and classical HLA I allele positive and negative cell lines) are used as well as others engineered by gene transfer (i.e. introducing HLA-E gene) or using techniques (i.e. CRISPR/Cas9) for developing cell lines with targeted gene deletions (i.e. TAP genes) that include creation of partial or full loss of function (LOF) mutations in genes.
Source human tumor cell lines for classical HLA, APM and non-classical HLAE expression at the mRNA and protein level in the absence and presence of IFN-gamma. For example, prostate tumor cell line PPC-1 under expresses MHC Class I and TAP-2 mRNA. LNCaP under expresses MHC class I but not TAP. HLA-E is detected using monoclonal antibody 3D12 (eBioscience).
Perform immunohistochemistry on freshly isolated human tumor tissue using antibodies to HLA-E and classical HLA alleles as well as antibodies to APM components. Parallel studies using PCR analysis are performed to same targets. Detergent solubilized HLA-E/peptide complexes from human tumor tissue are affinity purified and peptides contained in the HLA-E molecules are acid eluted for downstream analysis using standard LC/MS/MS techniques for peptide characterization.
Source normal human tissue, iPSC (inducible pluripotent stem cells) derived endothelial cells (HLA-E+) and PBMCs (HLA-E+) for HLA-E expression levels. For iPSC induced endothelial cells and PBMCs, HLA-E/peptide complexes are affinity purified from detergent solubilized membranes and peptides are eluted by acid treatment and assessed by standard LC/MS/MS techniques used for peptide characterization. This provides a baseline of peptide binders for HLA-E.
Transfect T2, K562 and other tumor cells with plasmid to express full-length (membrane anchored) HLA-E*01:01 or 01:03. LCL 721 cells express endogenous HLA-E*0103.
Generation of TAP-1 deficient cell lines. CPRISPR/Cas9 genome editing technology is employed to create targeted gene knockouts for TAP-1, tapasin and LMP2 (proteasomal defect) in a variety of tumorigenic and nontumorigenic human cell lines in order to suppress the conventional antigen processing pathway. The mutated cell lines expressing cell surface HLA-E are expanded to greater than 10¹¹ cells. HLA-E/peptide complexes from detergent solubilized cells are isolated using affinity capture chromatography and enriched HLA-E/peptide complexes are acid treated to release peptides for downstream LC/MS/MS evaluation. Peptide binders discovered for HLA-E are further validated in tumor tissue.
Transfect T2, K562, LCL 721.174 cells and other tumor cells with plasmid to express HLA-A*0201 or other classical HLA I allele. Selected cells are TAP-independent or are edited using CRISPR/Cas9 technology.
De novo discovery of neo-peptides (T-cell epitope impaired processing of presentation; TEIPP) presented by classical HLA alleles. Neo-peptides discovered from alternative processing routes that bind to HLA-A*0201 are identified using tumor cell lines engineered with defects in TAP1/2.

### Methodology for HLA-Elpeptide Target Discovery

HLA-I/peptide complexes were purified from 10¹¹ cells by affinity chromatography using antibody W6/32 after pre-clearing of the lysate with sepharose beads. After 10 kD filtration of acetic acid eluates, the complex peptide pool was fractionated using a 15 cm×200 cm RP-C18 column packed in house. The gradient was run from 0% to 50% solvent B (10/90/0.1, v/v/v, water/acetonitrile/formic acid) in 45 min. Fractions were injected onto a pre-column and eluted via an analytical nano-HPLC column. The gradient was run from 0% to 50% solvent B (10/90/0.1, v/v/v, water/acetonitrile/formic acid) in 90 min. The nano-HPLC column was drawn to a tip of approximately 5 μm and acted as the electrospray needle of the MS source. For mass spectrometry, a LTQ-FT Ultra mass spectrometer was used that was operated in data-dependent mode, automatically switching between MS and MS/MS acquisition. All fractions were twice recorded with a strict precursor mass tolerance (2 ppm) applying a SIM scan in the FTMS-measurements. Tandem mass spectra were matched against the IPIhuman v 3.72 using Mascot 2.2.04 (http://www.matrixscience.com) and sorted using Scaffold 2.2 (http://www.proteomesoftware.com). For the binding motif of HLA-E a more stringent method was performed in which only 8-13 mer peptides with a mascot ion score higher than 35 and a false discovery rate (FDR) of 5% were selected.

### Example 4

### Target Validation.

Part A—All peptides identified as binding to HLA-E*0101 and HLA-E*0103 in Examples 1 -3 are validated using tumor tissue or primary tumor cells and normal tissue or primary normal cells (i.e. cells derived from iPSC). The isolation methodology described in Example 3 is applied to extract HLA-E binding peptides from tumor and normal tissues/cells for analysis by LC/MS/MS. Peptides identified are compared to the peptides discovered in Examples 1-3.

Part B—In addition to a direct peptide confirmation approach using LC/MS/MS, HLA-E/peptide targets are also validated using specific TCR-like antibodies generated by immunizing animal with the specific HLA-E/peptide monomer (see Example 5). For this, tissue or cells are prepared for staining using standard sample processing and preparation protocols for using TCR-like antibodies made to a specific HLA-E/peptide target.

Immunocytochemistry experiments are performed with the HLA-A2 and/or HLA-E*0101 or HLA-E*0103 positive cancer cell line using the TCR-like antibody and murine isotype control Abs. Cytospin-prepared and methanol (5%) fixed cancer cells (53104) are incubated with TCR-like antibody at a concentration of 0.5 mg/ml for 30 min, washed, and incubated for 30 min with Ab conjugate goat antimouse—rhodamine (Millipore, Bedford, MA) at 0.5 mg/ml. Stained samples were analyzed for fluorescence using a Nikon Eclipse TE 2000, inverted, deconvolution microscope with Simple PCI Suite software (Nikon, Melville, N.Y.). DAPI (Vector, Burlingame, Calif.) was used as a counter stain for the nucleus.

Tumor samples from each patient were placed in Cryomold (Fisher Scientific, Pittsburgh, Pa.), covered in OCT media, flash frozen using isopentane and dry ice, and stored at 280° C. until used. Tissue sections were made at 5-mm size and fixed using 5% methanol and stained with TCR-like antibody and control antibody at 1 mg/ml for 1 h in diluent containing 1.0% horse serum to prevent nonspecific staining of tissue. Detection of primary Ab binding was determined using goat anti-mouse Ig-HRP (ImmPRESS Anti-Mouse Ig-peroxidase Kit, Vector) that, in the presence of substrate chromagen (3,39 diaminobenzidine [DAB]; Vector), provides an indicator system (formation of brown precipitate) to visualize the location of Ag/Ab binding using light microscopy. Hematoxylin QS was used as a nuclear counterstain (Vector). H&E stains (Sigma-Aldrich, St. Louis, MO) were used to assess cell morphology and tumor cell presence in tissue. Tissue sections were analyzed using light microscopy (Nikon Eclipse TE 2000, inverted, deconvolution microscope with Simple PCI Suite software).

Scoring TCR-like antibody staining of human tissue to accurately reflect total cell staining and intensity, an in-house scoring protocol for TCR-like antibody was implemented and followed. In this manner, a screening method consisting of proportion of stain (0-4) and intensity of stain (0-4) was established. A proportion of stain score of 0 represents no stain, a 1+ represents an average of 1-25 cells stained positive of 100 cells in the field (1-25%), a 2+ represents an average of 26-50 cells of 100 cells (26-50%), a 3+ score represents an average of 51-75 cells of 100 cells (51-75%), and a 4+ represents an average of 76-100 cells of 100 cells stained (76-100%). Intensity scores were based on a scale of 0-4, representing degrees of brown precipitate formed, in which 0 is negative, 1+ is weakly brown, 2+ is intermediately brown, 3+ is strongly brown, and 4+ is very dense brown. Finally, a total score (0-8) was determined by adding the scores for the proportion of stain and intensity of stain. Tissue sections were stained with TCR-like antibody and isotype controls at 1 mg/ml. The scores for proportion of stain and intensity of stain were reported as averages from five fields for each tissue sample.

### Example 5

### Production of Biotinylated MHC-Peptide Complexes.

Soluble MHC class I/peptide complexes were generated by overexpression of the HLA-A2 heavy chain (HC) and beta 2 microglobulin as recombinant proteins in E. coli and subsequent in vitro refolding and assembly in the presence of 10uM of specific peptide. To obtain soluble MHC/peptide complexes the HC sequence was mutagenized to remove the cytosolic and transmembrane regions. In order to specifically biotinylate refolded, monomeric MHC/peptide complexes, the HC was expressed as a fusion protein containing a specific biotinylation site at the C-terminus. These short sequences are sufficient for enzymatic in vitro biotinylation of a single lysine residue within this sequence using the biotin protein ligase BirA.

### Example 6

### The Immunogen for Immunization.

The T-cell receptor-like antibody is produced by a method that includes identifying a peptide of interest, wherein the peptide of interest is capable of being presented by an MHC I molecule and in particular is a peptide/HLA-E complex, and wherein the vaccine composition comprises the peptide of interest. An immunogen comprising a monomer of one peptide/MHC complex is then formed, wherein the peptide of the peptide/MHC complex is the peptide of interest. An effective amount of immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced, wherein the desired antibodies differentiate the peptide/MHC complex from the MHC molecule alone, the peptide of interest alone, and a complex of MHC and irrelevant peptide. The B-cells are then isolated from immunized animal and an antibody library is constructed using bacteriophage or yeast or other display systems.

An effective amount of immunogen is formed using peptide/HLA-E tetramers formed using biotinylated monomers with avidin or derivatives of avidin such as streptavidin and neutravidin to form tetramer complexes of peptide/HLA-E. The immunogen is prepared with adjuvant, for example Quil-A, and subcutaneously administered to animals for eliciting an immune response, wherein the immunogen retains a three-dimensional form of the peptide/HLA-E complex thereof for a period of time sufficient to elicit an immune response against the three-dimensional presentation of the peptide in the binding groove of the HLA-E MHC I molecule. The B-cells are then isolated from immunized animal and an antibody library is constructed using bacteriophage or yeast or other display systems.

### Example 7

### Library Construction and Selection of Phage on Biotinylated Non-Classical and Classical HLA/Peptide Complexes.

Phage display libraries are made from immunized mice and llamas. Immunized libraries of scFv or single domain V_{H}H antibodies are constructed by reverse transcription and polymerase chain reaction, a library of scFv or single domain antibodies containing 100-1000 million clones is regularly produced. All antibody libraries express scFv or single domain V_{H}H antibodies as pIII fusion in phagemid. The M13KE phage is used to generate phage particles for biopanning. scFv and single-domain V_{H}H phage display libraries contain approximately 1x10⁹ independent clones and are used for selection.

Selection of Phage on HLA-E*0101, HLA-E*0103 and HLA-A*0201/Peptide YLLPAIVHI (SEQ ID NO: 17) from Human p68 RNA Helicase Complex (Irrelevant Peptide)

Phage were first preincubated with streptavidin paramagnetic DYNABEADS (30 ul; Dynal, Oslo, Norway) and 150 ug unbiotinylated HLA-A2-I/YLLPAIVHI or HLAE* 0101/*0103/YLLPAIVHI (irrelevant complex) in 1 ml of PBS to remove any phage which expressed an antibody that binds to streptavidin or the general framework of HLA-A2 and HLA-E.

The DYNABEADS were subsequently captured using a magnet and the supernatant (phage and irrelevant complex mixture) transferred to a separate tube containing 7.5 μg of biotinylated HLA-A2/YLLPAIVHI or HLA-E*0101/*0103/ YLLPAIVHI (from human p68 RNA helicase) and 7.5 ug of biotinylated HLA-A2-KVAELVHFL peptide or HLA-E*0101/*0103/ KVAELVHFL (MAGE-A3) and incubated at RT for 1 hour. The final mixture (1 ml) was then added to 200 μl of DYNABEADS (preincubated with 2% Milk and washed with PBS) and the contents were mixed for 15 min. at RT with continuous rotation. The beads were then washed 10 times with PBS/0.1% TWEEN and 3 times with PBS and the bound phage were eluted from the DYNABEADS using 1 mg/ml trypsin in PBS (0.5 ml) for 15 min. at RT.

The phage is then used to infect ER2738 E. coli (growing in log phase) at 37 degree C. in 20 ml of LB for 1 hour. 1012 M13KE helper phage was subsequently added to the mixture, further incubated for an additional 30 minutes, and the cells pelleted using centrifugation (3000 rpm for 10 min.). The resulting cell pellet was resuspended in 200 ml LB+Ampicillin (100 μg/ml)+Kanamycin (50 μg/ml) and incubated overnight at 30 degree C.

The following morning, the overnight cultures were centrifuged at 3000 rpm for 15 min. and the supernatant (180 ml) was mixed with polyethylene glycol (PEG) on ice for 1 hour so as to precipitate the amplified phage from the previous round of selection. The PEG/phage mixture was then centrifuged at 3000 rpm for 20 min., and some of the resulting phage pellet used for subsequent rounds of panning while the rest was frozen down in 15% glycerol at −80 degree C. Subsequent rounds of panning were done using the same protocol as above with an increase in DYNABEAD washing steps and a decrease in the amount of biotinylated complexes used for selection.

After the final round of antibody selection, the eluted phage are used to infect both ER2738 and HB2151 E. coli; ER2738 cells were cultured overnight as mentioned above while the HB2151 cells were plated on TYE+Ampicillin (100 μg/ml) agar plates. The next morning, individual colonies from the agar plate were picked and used to inoculate individual wells of a 48-well plate containing 400 ul LB+Ampicillin (100 μg/ml)/well. After incubation for 3-6 hours at 37 degree C., 200 ul of 50% glycerol solution was added to each well and the plates stored at −80 degree C. as monoclonal stock cultures. Selection of Phage on HLA-A2-peptide and HLA-E*01011*0103 specific peptide complexes

Selection is performed similarly to the method above with slight modifications. 3×1012 Phage is first preincubated with streptavidin paramagnetic DYNABEADS (50 ul; Dynal, Oslo, Norway) and 20 unbiotinylated HLA-A2-YLLPAIVHI or HLA-E/YLLPAIVHI peptide (irrelevant complex) in 1 ml of PBS to deplete the streptavidin and HLA-A2 or HLA-E binders. The DYNABEADS is subsequently captured using a magnet and the supernatant (phage and irrelevant complex mixture) transferred to a separate tube containing 5 μg of biotinylated HLA-A2/specific peptide or HLA-E*0101/*0103/specific peptide and incubated at RT for 1 hour. The final mixture (1 ml) was then added to 100 μl of DYNABEADS (preincubated with 2% Milk and washed with PBS) and the contents were mixed for 30 min. at RT with continuous rotation. The beads were then washed 10 times with PBS/0.1% TWEEN and 3 times with PBS and the bound phage were eluted from the DYNABEADS using 1 mg/ml trypsin in PBS (0.5 ml) for 20 min. at RT. All subsequent steps were performed as above.

Unique clones are identified from more than 1 million binders by deep sequencing using NGS technology. Up to 10,000 unique binders are expressed using cell-free extracts and then screened using ResoSens label-free technology to perform high throughput screening to rank-order specific binders based on binding kinetics.

### Example 8

### Selectivity Assays.

The top 1,000 binders determined from the primary screen and rank-order filing are tested for binding selectivity in a high throughput screening assays with immobilized monomers of HLA-A2/peptides or HLA-E/peptides (>1,000 irrelevant random peptides) on the surface of the bionetic plate. Antibody binders are characterized for off-target reactivity to >1,000 different peptide/HLA-A or HLA-E targets immobilized on 96-well plates. The specific antibody binders are added to each well containing monomers of irrelevant peptide/HLA-E complexes and real-time binding is observed using label-free technology. Candidate antibody binders that do not bind to monomers of irrelevant peptide/HLA-E complexes are selected for further analysis.

### Example 9

Expression and Purification of Soluble scFv-Fc Fusion Protein.

Supernatant containing the soluble scFv-Fc fusion protein or the single domain V_{H}H-Fc fusion protein are purified using Protein A/G affinity chromatography medium (GE Healthcare). First, 1.5 ml of Protein A/G resin is loaded onto a column and activated with 20 ml of PBS. The supernatant is loaded onto the column using a peristaltic pump at a flow rate of approximately 1 ml/min. The column is subsequently washed using 40 ml of PBS until the flow-through registered an OD280 of less than 0.05. The scFv-Fc fusion protein is then eluted from the resin using 10 ml citrate buffer (pH 2.0) and directly into 10 ml of 1 M Tris for neutralization. The eluted scFv-Fc is subsequently concentrated using a 50,000 MWCO VIVASPIN centrifuge tube (Sartorius Stedim) and tested for its ability to bind to recombinant antigen using ELISA and the BIACORE Tw00 (GE Healthcare) as well as detect peptide pulsed T2 cells expressing HLA-E or classical HLA I alleles on the cell surface using flow cytometry.

### Example 10

### Binding Kinetics Analysis.

A second round of kinetic measurements are performed by surface plasmon resonance using the BIACORE T200 (GE Biosciences). Briefly, the first two flow cells of a CMS chip (GE Biosciences) are activated using the standard amine coupling reagents in HBS-EP running buffer (0.01 M HEPES, 0.15 M NaCl, 3 mM EDTA, 0.005% TWEEN 20) with flow cell 2 immobilized with the purified clones of scFv-Fc or single domain Fc fusion proteins. Subsequently, the target of antibody clones, HLA-E*0101 or HLA-E*0103 and peptide monomers as well as HLA-A2-peptide monomer (222 nM-13.875 nM) are injected over both the 1st (reference) and 2^{nd} flow cells at 20 μl/min for 120 sec, followed by the addition of running buffer for an extra 180 sec. Kinetics values were determined using the BIACORE T200 Evaluation Software 2.0 and 1:1 binding model (local Rmax).

### Example 11

### Flow Cytometry.

Peptide-pulsed T2 cells are transferred to plastic polystyrene round-bottom tubes (Becton Dickinson Labware) and washed with PBS. The cells are subsequently incubated with 5 ug of either targeted or non-specific purified scFv or scFv-Fc or single domain V_{H}H or single domain-Fc antibody on ice for 40 min. The cells are washed with PBS and then incubated with 1 ug of biotinylated mouse-anti-myc antibody (Clone 9E10; Sigma Aldrich) or biotinylated mouse-anti-human IgG Fc specific (Jackson Immunoresearch Laboratories) on ice for 30 mM. The cells are washed with PBS and then incubated with streptavidin-PE (BD Biosciences). Lastly, the cells are washed once more with PBS and analyzed on the BD FACS Calibur.

### Example 12

### Epitope Mapping Using Alanine Scanning.

Antibody binders with high affinity >10 nM for cognate HLA-E/peptide are mapped for binding preference to alanine substituted peptide/HLA-E complexes.

### Example 13

### Bispecific TCR-Like Antibodies.

A TCR-like antibody clone, designated as RAH TCR-like antibody against RAH (49-57)/H-2Db complex was generated. RAHYNIVTF (SEQ ID NO: 40) (49-57) peptide has been demonstrated as an immune dominant epitope of the HPV E7 protein and is known to be presented in the context of mouse MHC I, H-2D. This peptide has been directly identified on the surface of murine tumor cells. The RAH TCR-like antibody has been characterized and validated for its ability to react only to relevant peptide/MHC I (RAH (49-57)/H-2Db) complexes but not to any other peptide/MHC I complexes using ELISA and flow based assays. In addition, for the first time, utilizing this TCR-like antibody the levels of naturally processed RAH (49-57)/H-2Db complex on the surface of TC-1 and C3.43, murine tumor cells were interrogated.

The heavy and light chains of RAH TCR-like antibody were cloned. Briefly, mRNA was isolated from RAH hybridoma cells and PCR was performed using the following Light chain/Heavy chain variable region primers (US Biologicals) to murine antibody sequences. The primers used were Light chain forward: LCVP-K-5 GACATTGTGATGACCCAGTCT (SEQ ID NO: 41) and Light chain reverse: LCCP-K-1 GGATACAGTTGGTGCAGCATC (SEQ ID NO: 42) and for the heavy chain we used forward: HCVPCAGGTGCAGCTGAAGCAGTC (SEQ ID NO: 43) and Heavy chain reverse: HCCP-1GGCCAGTGGATAGTCAGATGGGGGTGTCGTCGTTTTGGC (SEQ ID NO: 44). The VL & VH genes were joined together to form a RAH scFv using Splicing over extension PCR. Light chain and heavy chain linked together to form single chain variable fragment (scFv) by overlapping extension PCR using the following external primers:

```
Light chain variable primer (No. 5) with Hind III
(SEQ ID NO: 45)
5′--AGTCAT--AAGCTT--GAC ATT GTG ATG ACC CAG TCT--3′
            Hind III
Heavy chain constant primer with Xho I
(SEQ ID NO: 46)
5′--AGTCAT-CTCGAG-GGC CAG TGG ATA GTC AGA TGG--3′
            Xho I
```

The amplified fragments are ligated into pGEM-T Easy Vector system transformed into E. coli and clones were picked for sequence analysis to identify authentic sequence.

### Construction of Bispecific T-cell Engager

The RAH scFv & murine anti-CD3 scFv were cloned into pSecTag2 Hygro B vector. The assembled bispecific plasmid was used to transform NEB 5-alpha competent E. coli cells (NEB). Transformed colonies were picked from LB plate containing ampicillin, expanded and sent off for sequencing verification (plasmid containing IgGk leader sequence, bispecific antibody and Myc/6× His tag (SEQ ID NO: 12)). The bispecific RAH TCR-like antibody scFv×anti-CD3 scFv in pSecTag2 vector was used to transfect CHOK1 cells using electroporation (Nucleofactor kit Lonza VCA-1003).

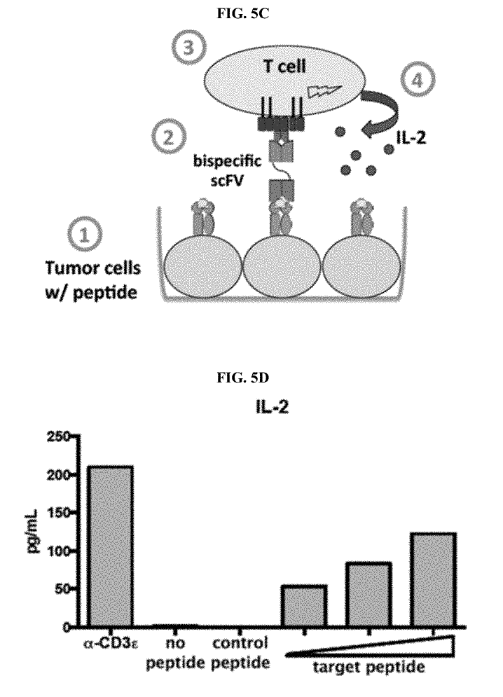

Bispecific antibody was purified using a 6-His (SEQ ID NO: 12) affinity column and the purified bispecific antibody was assessed for activity in vitro. As exemplified in FIG. 5, a bispecific scFv with RAH TCR-like targeting and a mouse CD3e binding motif activates T cells.

### Example 14

### TCR-Like Immunotoxin/ADC Efficacy is Dependent on Target Copy Number.

The MDA-MB-231 cell line was analyzed for specific peptide/HLA copy number expression specific to mRL6A and mRL21A binding. MDA-MB-231 cell killing appeared to be linked to copy number expression.

To study the role of a TCR-like antibody as an antibody drug-conjugate and correlate its activity to target copy number, MDA-MB-231 cells were peptide-pulsed with 1, 10, and 20 μg/ml of the HLA-A2 peptide KIFGSLAFL (SEQ ID NO: 63) (KIF) over a 3 hour period. MDA-MB-231 cells do not naturally exhibit strong reactivity against the TCR-like antibody. Mab-Zap was employed as a secondary ADC strategy to peptide-pulsed MDA-MB-231 cells. As more tumor cell peptide/HLA targets were specifically bound by the TCR-like antibody, cell viability decreased, although EC50 values were similar at 0.102 nM and 0.122 nM for 1 μg/ml KIF and 20 μg/ml KIF peptide loading, respectively. Under peptide-pulsing conditions of 1 μg/ml KIF, approximately 21,000 molecules of TCR-like antibody bound MDA-MB-231 cells and reduced viability near 80% through inhibition of protein synthesis. Once TCR-like antibody bound tumor cells over 47,000 molecules per cell, target viability dropped to around 60%.

To determine the minimum surface-bound peptide/HLA-A2 expression required to induce target cell death in vitro using a TCR-like antibody directly conjugated to a potent small molecule drug, a TCR-like antibody was linked to the DNA alkylating molecule duocarmycin and incubated alongside MDA-MBA-231 cells peptide-pulsed with various concentrations of the KIF peptide. As indicated, MDAMBA-231 cells harboring as low as 350 KIF/HLA-A2 molecules per cell could be readily killed in culture. Altogether, these results confirm the importance of TCR-like immunotoxins/ADCs binding a threshold level of target peptide/HLA to incite observable tumor cell death. Select TCR-like ADCs were also adept at minimizing tumor growth when targeting physiological levels of TA/HLA complexes (<1,000 copies per cell) routinely observed in clinical specimens.

### Example 15

### Discovery and Validation of Peptides Binding to HLA-E in Cancer Cells.

A two-step process was used for discovering and validating neo-peptides bound to HLA-E in cancer cells. An in silico discovery method was used to predict neo-peptides derived from alternative antigen processing pathways for loading peptides into HLA-E complexes. The identified targets were then directly validated using patient-derived xenograft (PDX) tissue.

Previously reported peptide sequences, which include HLA-E binding peptides reported from cancer cells and infectious agents, along with peptides predicted to bind to HLA-E using a newly developed predictive algorithm that models the HLA-E peptide binding pocket were used. Though several algorithms are available in the public domain (i.e. NETMHC4.0 (http://www.cbs.dtu.dk/services/NetMHC-4.0/) and IEDB (http://www.iedb.org) and are useful in predicting peptides binding to classical HLA-I alleles, predicting with high accuracy peptides binding to HLA-E in cancer cells is challenging and required development of new predictive algorithms.

### Novel Approach for Predicting Cancer Peptides Binding to HLA-E—Protein Selection Applied to Cancer

In brief, mRNA expression data was downloaded from the Cancer Cell Line Encyclopedia (CCLE) and for each cancer tissue, genes that showed an average expression across cell lines >=9 (range 8 to 12) were retained. Gene expression data and clinical data were obtained from The Cancer Genome Atlas (TCGA), and patient information was matched with gene expression for several cancer types (lung, breast, colorectal, melanoma and liver). Using TCGA data, survival analysis was performed for all genes with Cox regression and for each cancer type genes were selected with a hazard ratio (HR) >=1.1 and a p-value <=0.05. CCLE and TCGA genes were then crisscrossed so that the final list only had highly expressed genes in cancer cell lines predicting poor patient outcome. From the chosen genes, a list of reviewed human protein sequences was downloaded from Uniprot.

### Protein Docking and Ranking of Peptides

Following the Internal Coordinate Mechanics (ICM) docking method, a virtual screening and scoring of each classified binder was performed. ICM' s scoring function provides a good approximation of the binding free energy between a ligand and a receptor, and is a function of different energy terms based on a force-field. The function is weighted per the following parameters (i) internal force-field energy of the ligand, (ii) entropy loss of the ligand between bound and unbound states, (iii) ligand-receptor hydrogen bond interactions, (iv) polar and non-polar solvation energy differences between bound and unbound states, (v) electrostatic energy, (vi) hydrophobic energy, and (vii) hydrogen bond donor or acceptor desolvation. The lower the ICM score, the higher the chance the ligand is a binder.

Docking simulation using the ICM score to rank HLA-E binding peptides was tested with the peptide VMAPRTLIL (SEQ ID NO: 13) found in literature and IEDB and HLA-E binders derived from Mycobacterium tuberculosis (Table 2). The VMAPRTLIL (SEQ ID NO: 13) having the optimal free energy of binding (most negative ICM score) fit best in the binding pocket with docking simulation experiments.

TABLE 2
ICM score of peptides derived from human HLA-C and Mycobacterium tuberculosis.

| | Hydro | | | | | |
|---|---|---|---|---|---|---|
| Sequence | index | Uniprot ID | Entry name | ICM Score | Gene | Organism |
| VMAPRTLIL | 1.467 | P04222 | 1C03_HUMAN | −28.154 | HLA-C | Homo sapiens (Human) |
| (SEQ ID NO: 13) | | | | | | |
| RMPPLGHEL | −0.589 | O53244 | O53244_MYCTO | −12.900 | MT3075 | Mycobacterium tuberculosis |
| (SEQ ID NO: 47) | | | | | | (strain CDC 1551/Oshkosh) |
| VMTTVLATL | 1.956 | P9WLS6 | Y1734_MYCTO | −2.995 | MT1774.1 | Mycobacterium tuberculosis |
| (SEQ ID NO: 48) | | | | | | (strain CDC 1551/Oshkosh) |
| RLPAKAPLL | 0.378 | P9WGR1 | INHA_MYCTU | 0.205 | inhA | Mycobacterium tuberculosis |
| (SEQ ID NO: 49) | | | | | | (strain ATCC 25618/H37Rv) |

### List of Predicted Peptides

Table 3 shows predicted peptides derived from proteins present in different cancer tissues after applying the protein selection method, proteasome degradation classification, HLA-E peptide classification and docking modeling. Besides general information on the protein and position of the peptide, the table contains ICM score, immunohistochemistry (IHC) data and proteomic data. IHC data was obtained from The Human Protein Atlas. IHC staining from breast, colorectal, lung, lymphoma, and skin cancer was also determined. The data shows number of cases detected in three levels high, medium and low or non-detected (ND). Proteomic data was downloaded from the NCI-60 proteome resource. The NCI-60 panel comprises 59 individual cancer cell lines derived from nine different tissues (brain, blood and bone marrow, breast, colon, kidney, lung, ovary, prostate, and skin), which were analyzed by different approaches including LFQ and iBAQ quantification of proteins on a log₁₀ protein intensity scale.

TABLE 3
Predicted peptide binders for HLA-E.

| | | | | Protein | | Hydro | Method | | |
|---|---|---|---|---|---|---|---|---|---|
| Protein | Sequence | Start | End | length | ICM | inded | of ID | TAP (+) | TAP (−) |
| EEF1E1 | SLLEKSLGL | 8 | 16 | 174 | −10.4361 | 0.6444 | MS, | Not | found |
| | (SEQ ID NO: 32) | | | | | | Prediction | found | |
| HSPD1/HSP60 | QMRPVSRVL | 10 | 18 | 573 | −4.9910 | −0.0889 | T-cell, | found | unknown |
| | (SEQ ID NO: 22) | | | | | | Prediction | | |
| CISD1 | WIAAVTIAA | 13 | 21 | 108 | −13.0877 | 2.0889 | Prediction | NA | NA |
| | (SEQ ID NO: 33) | | | | | | | | |
| HSPC185 | TSDMPGTTL | 14 | 22 | 236 | −9.8753 | −0.3000 | MS | Not | found |
| | (SEQ ID NO: 34) | | | | | | | found | |
| HEXB | MLALLTQVA | 26 | 34 | 556 | −13.6690 | 1.8778 | MS, | Not | found |
| | (SEQ ID NO: 35) | | | | | | Prediction | found | |
| YRDC | QMFEGPLAL | 185 | 193 | 279 | −8.0447 | 0.5667 | MS, | Not | found |
| | (SEQ ID NO: 36) | | | | | | Prediction | found | |
| STAT1 | VLWDRTFSL | 350 | 358 | 750 | −9.5114 | 0.4667 | MS | Not | found |
| | (SEQ ID NO: 37) | | | | | | | found | |
| PTPN2 | TLFFQQNAL | 407 | 415 | 415 | −3.7640 | 0.4222 | MS on | NA | found |
| | (SEQ ID NO: 38) | | | | | | HLA-A2 | | |
| CAD | GLADKVYFL | 445 | 453 | 2225 | −20.6331 | 0.8111 | MS, | Not | found |
| | (SEQ ID NO: 1) | | | | | | Prediction | found | |
| KIF11 | ILSPTVVSI | 639 | 647 | 1056 | −21.1171 | 1.9222 | Prediction | NA | NA |
| | (SEQ ID NO: 2) | | | | | | | | |

### Target Validation

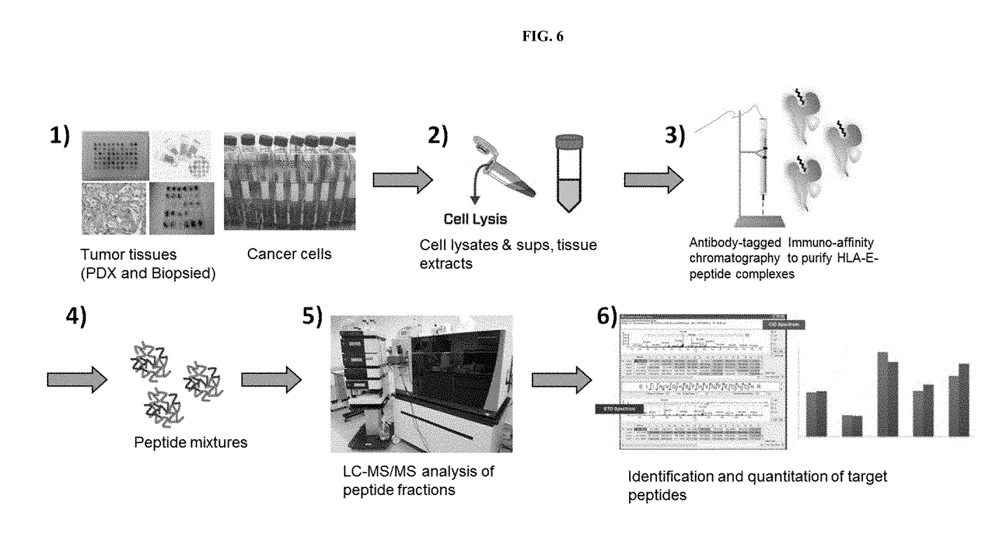

Predicted peptides shown in Table 3 were validated using patient derived xenograft (PDX) tissue samples. PDX tissue from a lung cancer patient was processed for isolation of HLA-E-peptide complexes for downstream analysis of peptides bound to the HLA-E complex (FIG. 6). The PDX model LU5139 had the following characteristics:

| | |
|---|---|
| Model ID: | LU5139 |
| Cancer Type: | LU-Lung Cancer |
| Subtype: | NSCLC, SCC |
| Ethnicity: | Caucasian |
| Gender: | F |
| Age: | 71 |
| Stage: | NA |
| Grade: | NA |
| Pathology Diagnosis: | squamous carcinoma |
| | moderately-differentiated invasive squamous |
| | cell carcinoma |
| Biopsy site: | Lung |
| Treatment history: | NA |
| Mouse Strain: | NOD SCID |

Additionally, the tumor tissue had the following HLA allotype: A*02:01, A*02:01, B*07:05, B*07:05, C*03:03 and C*07:02.

PDX lung tumor tissue was purchased from Crown Biosciences and used for extraction of HLA-E-peptide complexes. In brief, snap-frozen PDX tissue was added to 10 ml of lysis buffer (0.2 mM iodoacetamide, 1 mM EDTA, 1:200 dilution of protease inhibitor cocktail, 1 mM PMSF, and 1% octyl-β-D-glucopyranoside) and homogenized for 10 s on ice and incubated for 1 hr at 4 C. Following incubation, the sample was centrifuged for 20 min at 40,000 g to clarify the supernatant. An aliquot of sample supernatant was removed and protein concentration was determined by BCA assay.

The 4D12 hybridoma (ATCC), which produces a murine IgG1 monoclonal antibody specific for HLA-E, was used to generate an affinity column for enrichment of HLA-E-peptide complexes. Briefly, clarified supernatant from processed tumor tissue was added to column and continuously recirculated over column for 2 hrs at 4 C. Column was then washed with 1× PBS followed by 2 column volumes of sterile purified water (MilliQ water). The affinity column was then treated with 10 ml of 0.1 M Glycine buffer pH 3.0 and 1 ml sample aliquots were collected and assessed for protein. Samples were immediately neutralized with 0.1 ml of 0.1M NH₄HCO₃. Tubes containing protein sample were pooled and concentrated to <1 ml using filtration (5 kDa MW cutoff) and a desalting column prior to drying.

Peptide mixture samples underwent solid-phase extraction cleanup with an Oasis HLB elution plate (Waters) and the resulting samples were analyzed by LC/MS/MS, using an Orbitrap Fusion Lumos mass spectrometer (Thermo Electron) coupled to an Ultimate 3000 RSLC-Nano liquid chromatography system (Dionex). Samples were injected onto a 75 μm i.d., 50-cm long EasySpray column (Thermo), and eluted with a gradient from 0-28% buffer B over 60 min. Buffer A contained 2% (v/v) ACN and 0.1% formic acid in water, and buffer B contained 80% (v/v) ACN, 10% (v/v) trifluoroethanol, and 0.1% formic acid in water. The mass spectrometer operated in positive ion mode with a source voltage of 2.2 kV and an ion transfer tube temperature of 275° C. MS scans were acquired at 120,000 resolution in the Orbitrap and up to 10 MS/MS spectra were obtained in the ion trap for each full spectrum acquired using higher-energy collisional dissociation (HCD) for ions with charges 1-3. Dynamic exclusion was set for 25 s after an ion was selected for fragmentation.

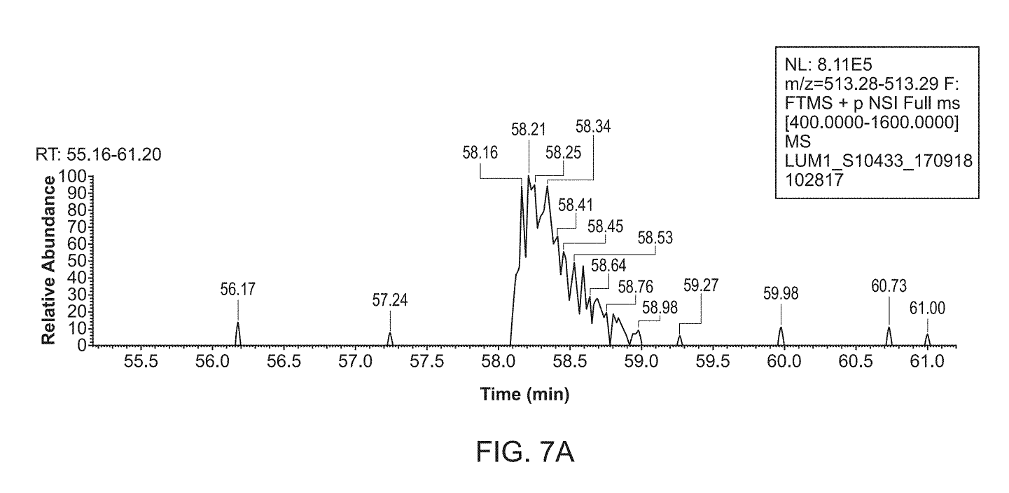

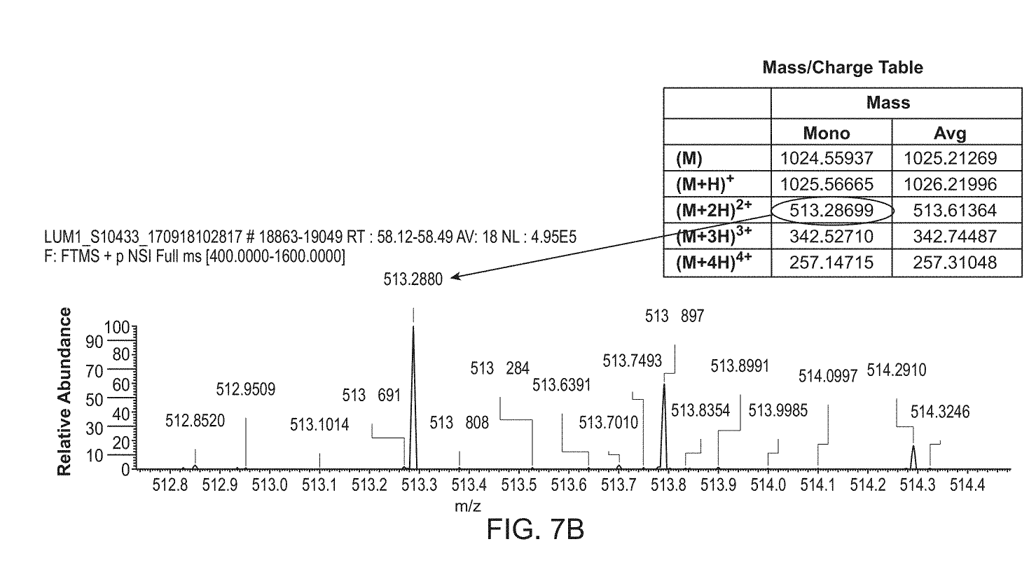

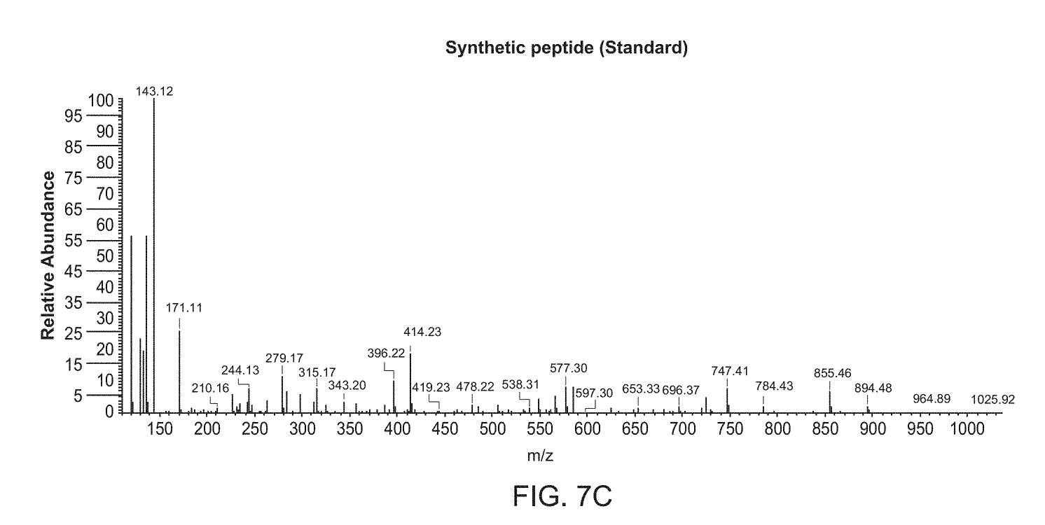

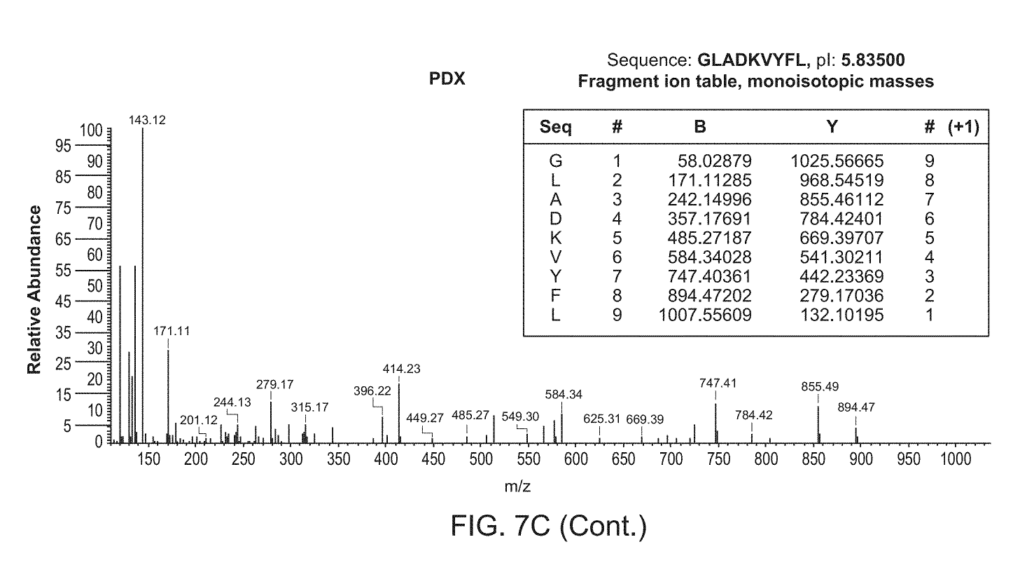

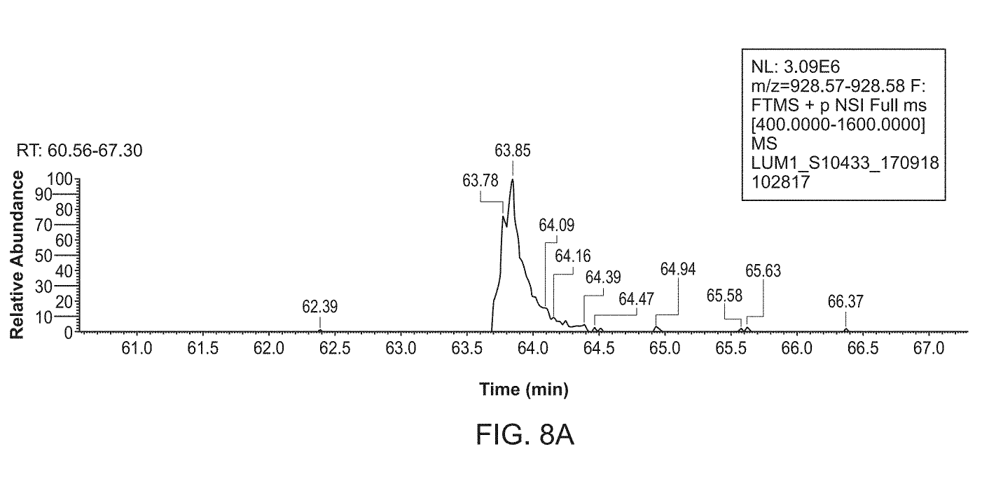

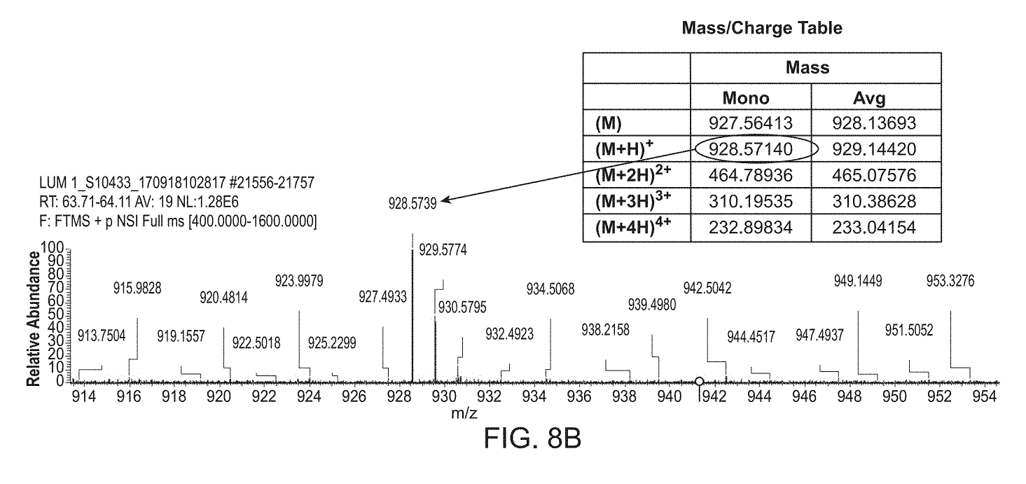

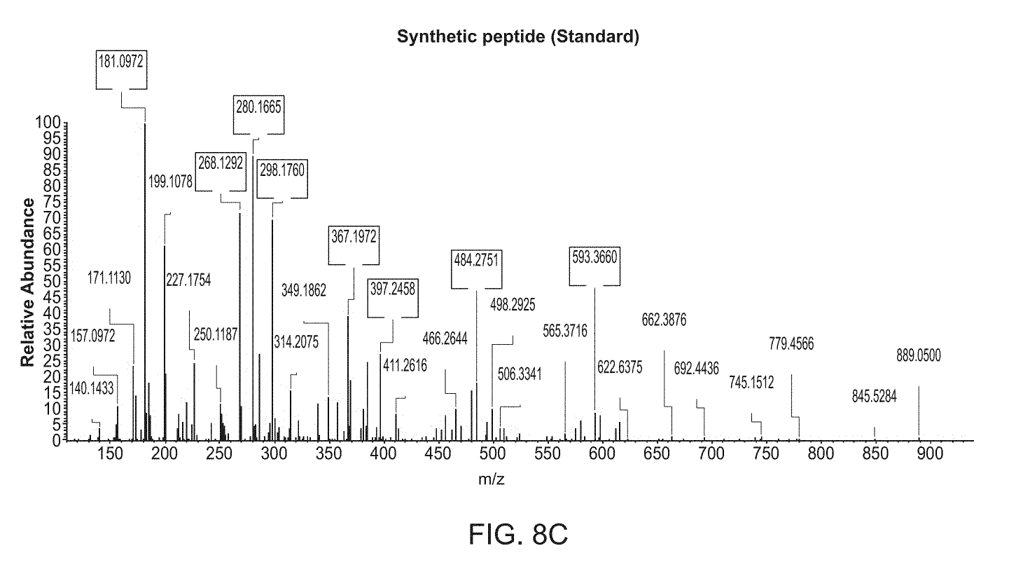

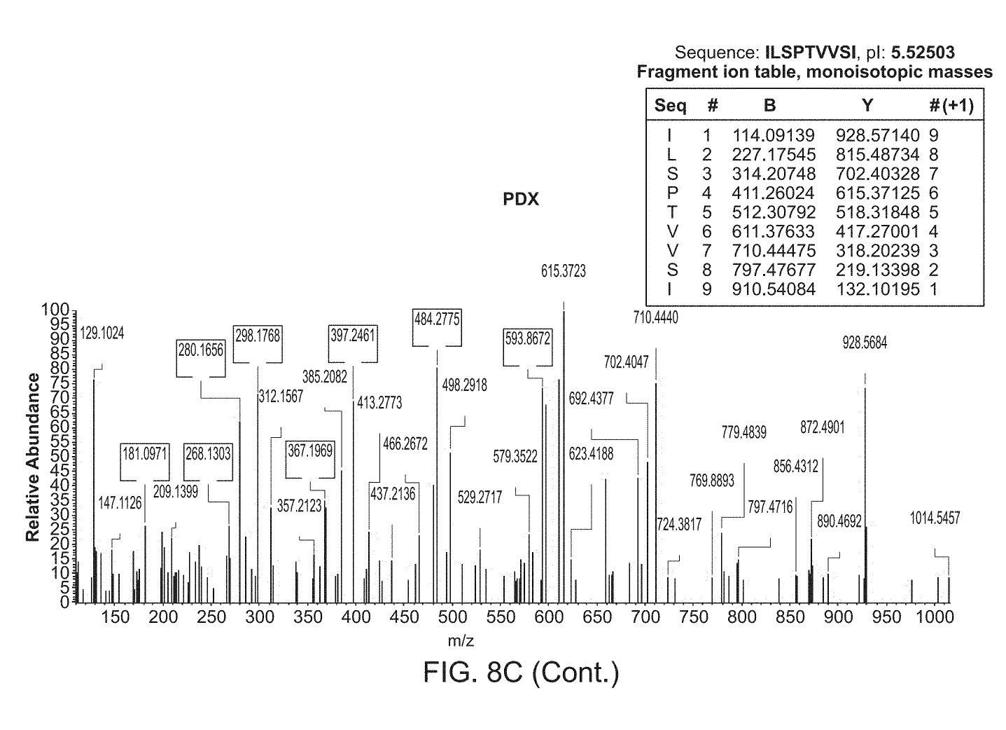

Shown in Table 4 are three peptides detected in lung PDX tissue. The LC/MS/MS results for peptides GLADKVYFL (SEQ ID NO: 1) and ILSPTVVSI (SEQ ID NO: 2) are shown in FIG. 7A-7C and FIG. 8A-8C, respectively.

TABLE 4
HLA-E predicted peptides validated using LC/MS/MS.

| | | | | Prediction | Prediction | | | | |
|---|---|---|---|---|---|---|---|---|---|
| | | | | score | score | | | | |
| Peptide | | | Protein | HLA- | HLA- | Hydro | Gene | UniProtKB/ | Method |
| Sequence | Start | End | length | E*0101 | E*0103 | index | Name | Swiss-Prot | of ID |
| MLALLTQVA | 26 | 34 | 556 | 4.397 | −13.6690 | 0.6444 | HEXB | P07686 | MS, |
| (SEQ ID NO: 35) | | | | | | | | | Prediction |
| GLADKVYFL | 445 | 453 | 2225 | −12.334 | −20.6331 | −0.3000 | CAD | P27708 | T-cell, |
| (SEQ ID NO: 1) | | | | | | | | | Prediction |
| ILSPTVVSI | 639 | 647 | 1056 | −8.494 | −21.1171 | 2.0889 | KIF11 | P52732 | Prediction |
| (SEQ ID NO: 2) | | | | | | | | | |

### Example 16

### Production of Recombinant HLA-E Monomers Containing Peptides.

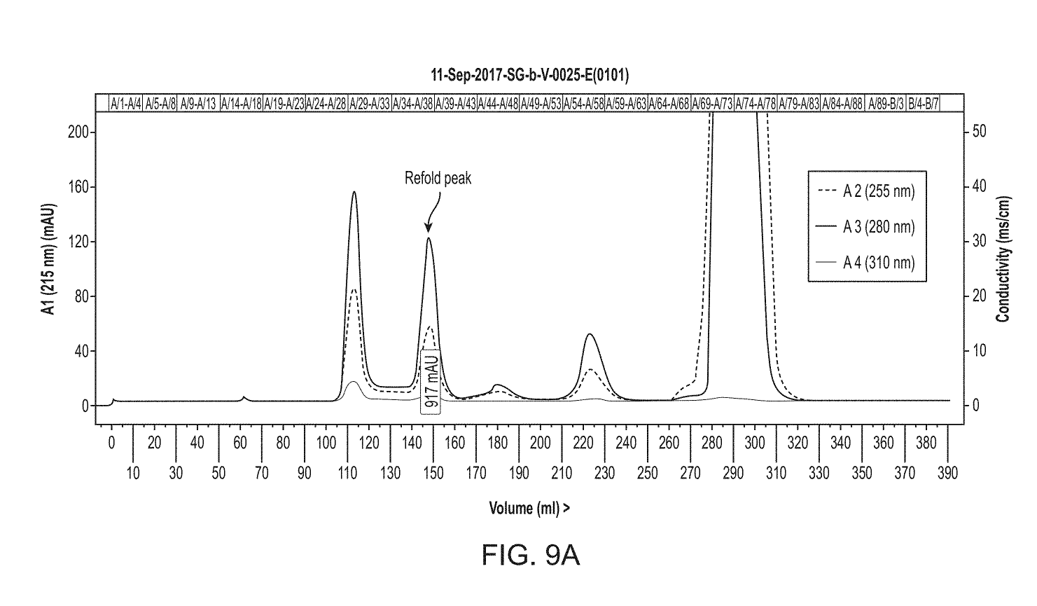

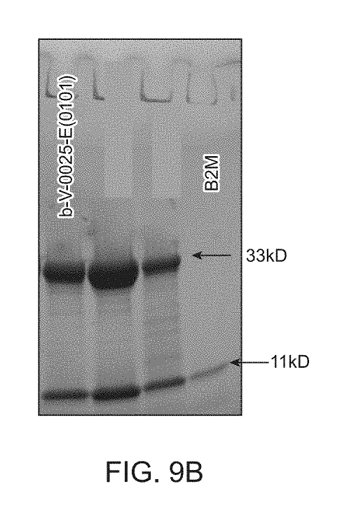

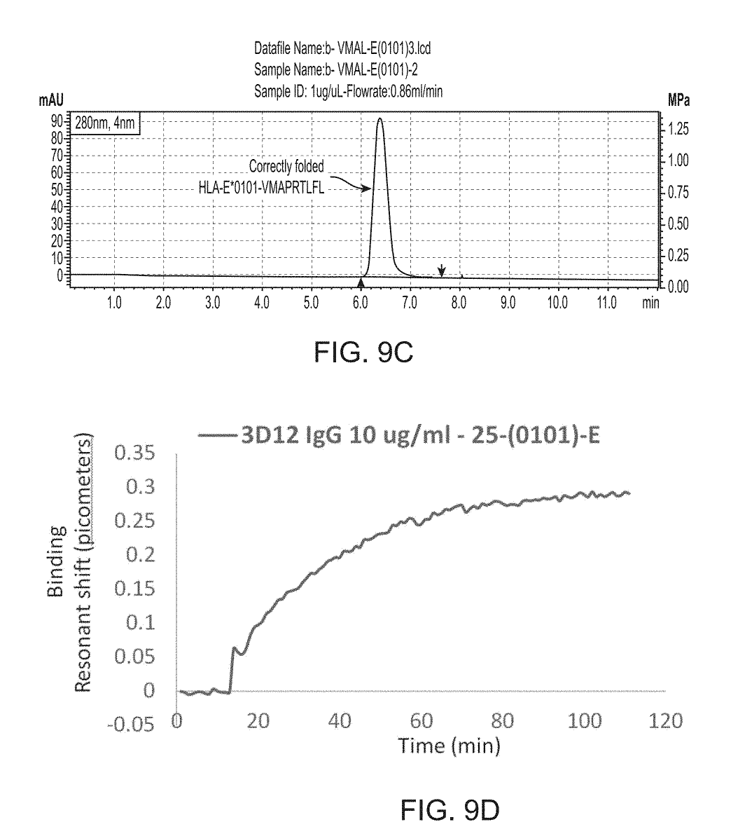

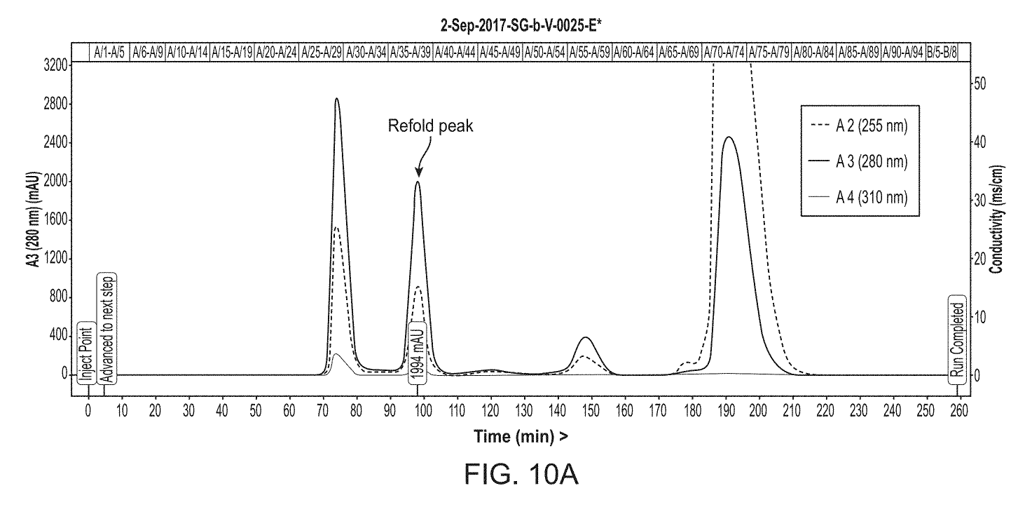

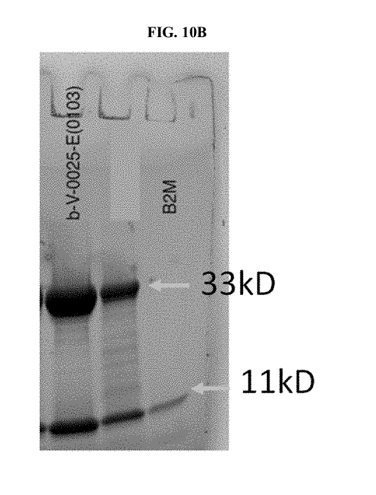

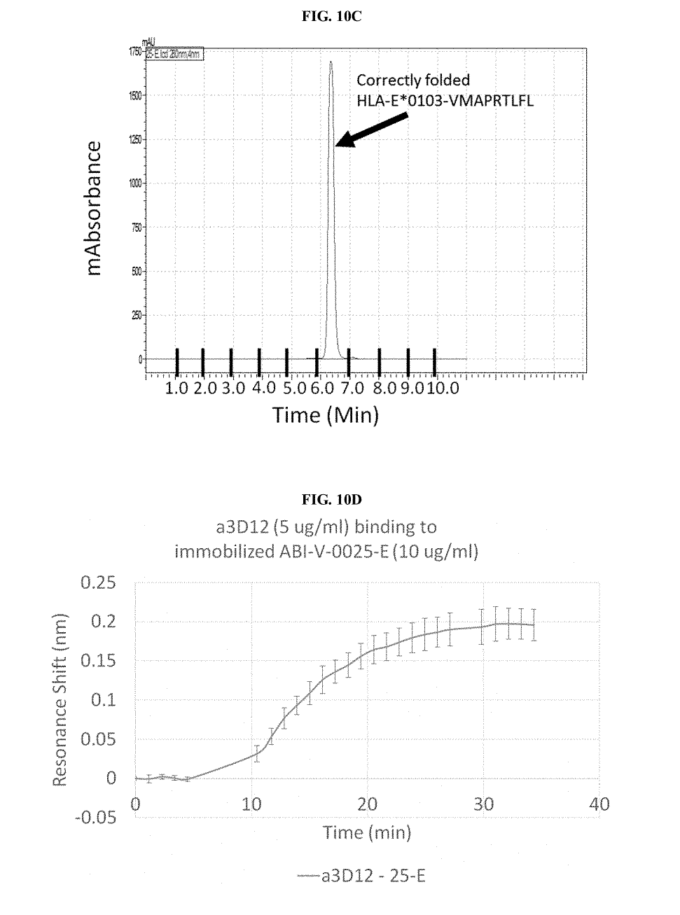

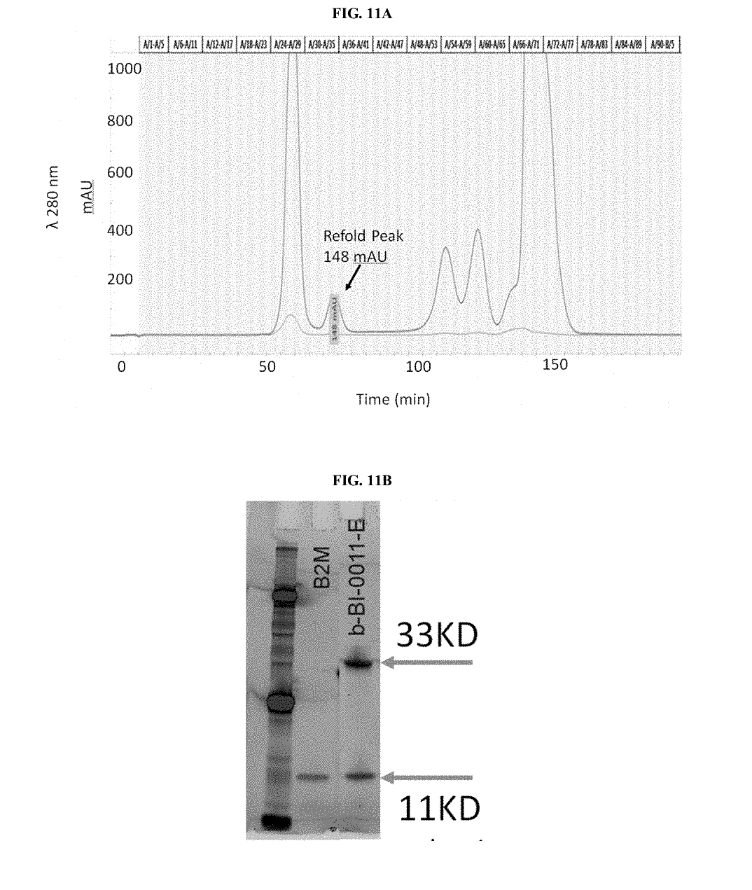

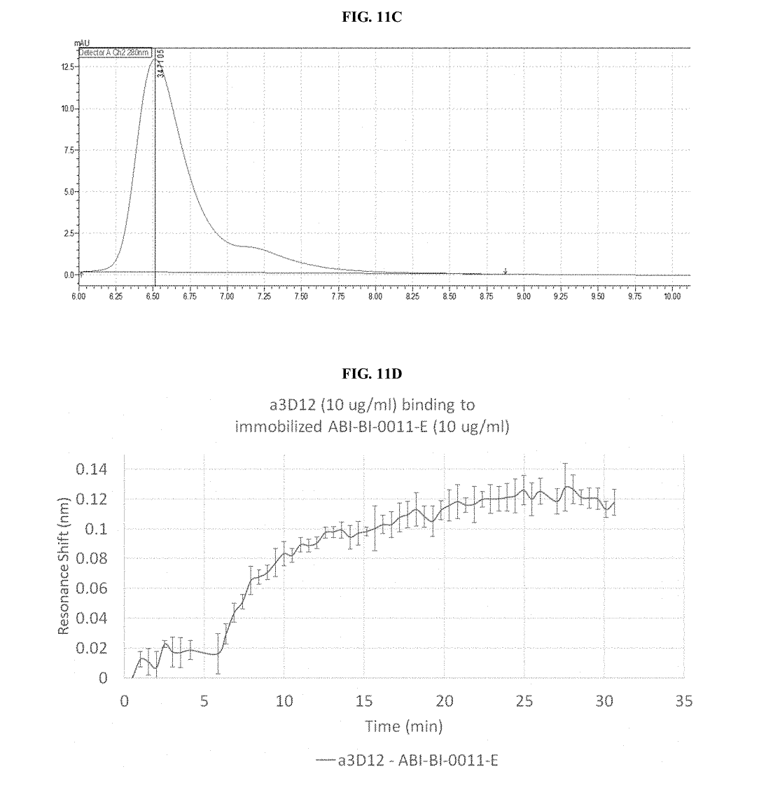

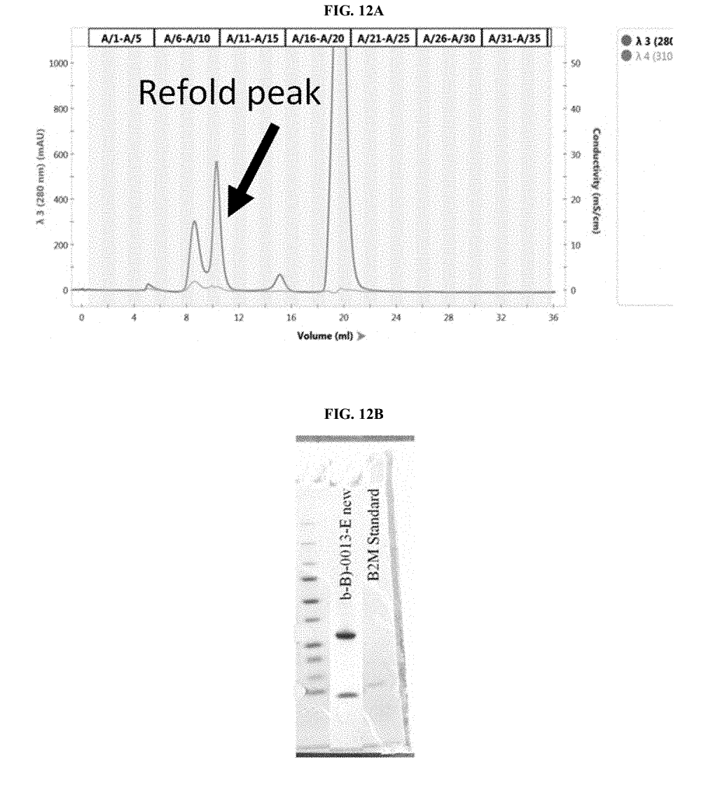

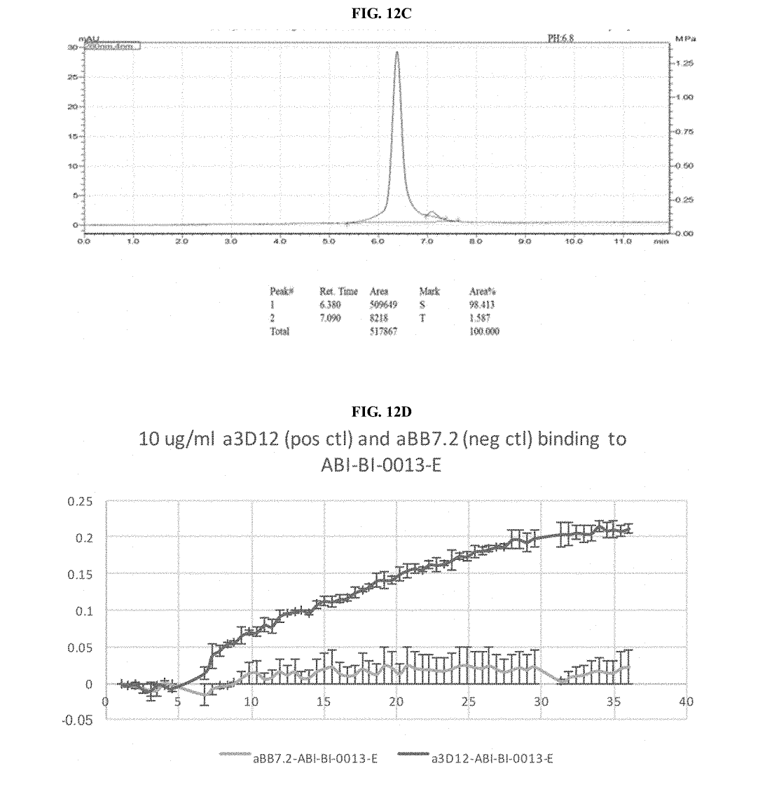

Beta-2-microglobulin (B2M) and the extracellular domains of HLA-E (*0101 and *0103) were produced as inclusion bodies in Escherichia coli and refolded with 10 μM of peptide. Soluble HLA-E-peptide complexes were obtained by mutagenizing the heavy chain gene sequence to remove the cytosolic and transmembrane regions. In order to specifically biotinylate refolded, monomeric HLA-E-peptide complexes, the heavy chain (HLA-E) was expressed as a fusion protein containing a specific biotinylation site at the C-terminus. These short sequences are sufficient for enzymatic in vitro biotinylation of a single lysine residue within this sequence using the biotin protein ligase BirA (Avidity, Col.). Refolded material was collected after running on a Superdex 75 sizing column using an NGC™ medium pressure liquid chromatography system (BioRad). In FIG. 9A, FIG. 10A, FIG. 11A, and FIG. 12A, the second peak from the chromatogram represents the correctly refolded HLA-E-peptide complex. Confirmation of properly refolded material (peak 2) by SDS-gel electrophoresis under reducing condition is shown in FIG. 9B, FIG. 10B, FIG. 11B, and FIG. 12B. Note two bands migrating at ˜33 kD and ˜11Kd indicating the presence of HLA-E heavy chain and B2M protein, respectively. HPLC analytical size exclusion chromatography (SEC) was then used to assess percent of material associated as an intact complex (HLA-E, B2M and peptide). The expected retention time for intact complex should be −6.384 minutes using the X-Bridge SEC. Finally, the refolded material was evaluated on the ResoSens label-free system (RSI, Arlington, Tex.). Neutravadin coated bionetic plates were then incubated with 10 μg/ml of biotin-labeled HLA-E-peptide complex. Correctly folded recombinant HLA-E-peptide was determined by binding of a conformation-dependent anti-HLA-E antibody, 3D12 (Abcam) to HLA-E—peptide complex immobilized on a label-free bionetic plate (RSI).

### Example 17

### Discovery of TCR-Like Antibodies to HLA-E-Peptide Complexes.

The T-cell receptor-like antibodies utilized in accordance with the presently disclosed and claimed inventive concepts(s) are produced by a number of methods that includes identifying a peptide of interest, wherein the peptide of interest is capable of being presented by an non-classical MHC I molecule and in particular is a peptide/HLA-E complex. The overall antibody discovery process used to generate antibodies to HLA-E-peptide complexes is exemplified in FIG. 13. Two standard in vitro display technologies, namely phage and yeast display, were used along with naive human and immune mouse and llama libraries. The selection process i.e. positive and negative selection and depletion and blocking molecules was optimized to discover binders to HLA-E-peptide targets of interest. Overview of standard protocol used for antibody phage display libraries:

Phage were first preincubated with streptavidin paramagnetic DYNABEADS (30 ul; Dynal, Oslo, Norway) and 150 ug unbiotinylated HLA-A2-peptide and HLA-E-peptide complexes (irrelevant complex) in 1 ml of PBS to remove any phage which expressed an antibody that binds to streptavidin or the general framework of HLA-A2 and HLA-E.

The DYNABEADS were subsequently captured using a magnet and the supernatant (phage and irrelevant complex mixture) transferred to a separate tube containing 7.5 ug of biotinylated HLA-E-peptide (HLA-E-peptide complex of interest) and incubated at RT for 1 hour. The final mixture (1 ml) was then added to 200 ul of DYNABEADS (preincubated with 2% Milk and washed with PBS) and the contents were mixed for 15 min. at RT with continuous rotation. The beads were then washed 10 times with PBS/0.1% TWEEN and 3 times with PBS and the bound phage were eluted from the DYNABEADS using 1 mg/ml trypsin in PBS (0.5 ml) for 15 min. at RT.

The phage were used to infect TG1strain of E. coli (growing in log phase) at 37 degree C. in 20 ml of LB for 1 hour. 10¹² M13KO helper phage was subsequently added to the mixture, further incubated for an additional 30 minutes, and the cells pelleted using centrifugation (3000 rpm for 10 min.). The resulting cell pellet was resuspended in 200 ml LB+Ampicillin (100 ug/ml)+Kanamycin (50 ug/ml) and incubated overnight at 30 degree C.

The following morning, the overnight cultures were centrifuged at 3000 rpm for 15 min. and the supernatant (180 ml) was mixed with polyethylene glycol (PEG) on ice for 1 hour so as to precipitate the amplified phage from the previous round of selection. The PEG/phage mixture was then centrifuged at 3000 rpm for 20 min., and some of the resulting phage pellet used for subsequent rounds of panning while the rest was frozen down in 15% glycerol at −80 degree C. Subsequent rounds of panning were done using the same protocol as above with an increase in DYNABEAD washing steps and a decrease in the amount of biotinylated complexes used for selection.

After the final round of antibody selection, the eluted phage were used to infect TG1 strain mentioned above while the HB2151 cells were plated on Ampicillin (100 ug/ml) agar plates. The next morning, individual colonies from the agar plate were picked and used to inoculate individual wells of a 48-well plate containing 400 ul LB+Ampicillin (100 ug/ml)/well. After incubation for 3-6 hours at 37 degree C., 200 ul of 50% glycerol solution was added to each well and the plates stored at −80 degree C. as monoclonal stock cultures.

Discovery of Antibody Binder from Pre-Made Human Semi-Synthetic Antibody Library:

A single-chain antibody (scFv) to HLA-E*0103-VMALRTLFL, a signal peptide derived from HLA-G was discovered and isolated from a phage display human semi-synthetic scFv library. A phage library using the mono display by pIX fusion was constructed in the scFv format using semi-synthesized VH and VL genes to create a total diversity of 1.42×10⁹. The library was propagated using E. coli TG1 host strain along with M13KO7 helper phage.

In-solution biopanning for target HLA-E*0103-VMALRTLFL: Just prior to working with the library, phage sample was precipitated, centrifuged for 10 min at 5,000 g and the pelleted sample was resuspended in 2%M-PBS. The library was then sequentially added to 1 ml—Dynabeads (MyOne Streptavidin T1) and after 1 hr incubation at room temperature the tube was placed in a magnetic rack for 1 minute to remove non-specific binding phage with beads. The aspirated supernatant containing phage was then added to beads containing blocking buffer, 2% M-PBS and step from #1 (incubation with 1 ml—Dynabeads (MyOne Streptavidin T1)) was repeated. Finally, the supernatant containing phage was mixed with streptavidin coated beads and incubated with 4 biotinylated targets (all HLA-A2-peptide complexes) to deplete non-specifically binding phage. Included in step 3 was unbiotinylated HLA-A2-peptide that was used as a blocking reagent.

To isolate scFv phage particles with target specificity, streptavidin beads were coated with biotinylated HLA-E*0103-VMALRTLFL. Included in the biopanning step was the addition of unbiotinylated random HLA-A2-peptides used as blocking molecules. The enriching factor was 2.73×10⁶ for the 1^{st} round of biopanning against the target, HLA-E*0103-VMAPRTLFLF.

The output phage was amplified and subjected to the 2^{nd} round of biopanning. The depletion and pre-blocking steps were performed as before using the a mixture of HLA-A2-peptide complexes and then the library was screened against HLA-E*0103-VMAPRTLFL. The enriching factor was 4.03×10² for the 2^{nd} round of biopanning against the target. In addition, the target screening showed a difference with the HLA-E negative control (HLA-E*0103-YLLPAIVHI) and wells not coated with any target.

To further enrich for phage antibodies binding to the target antigen, a 3^{rd} round of biopanning was performed. An enriching factor of 2.31×10² was obtained compared to the negative controls (HLA-E*0103-peptide) and no coating. Moreover, a polyclonal phage ELISA was performed using as targets HLA-E*0103-VMAPRTLFL and HLA-A2-peptide mix. The wells coated with HLA-E*0103-VMAPRTLFL had a much greater OD450 nm value than the wells coated with HLA-A2-peptide mix suggesting successful enrichment of scFv phage binding to target, HLA-E*0103-VMAPRTLFL peptide.

A final round of biopanning was performed with the modification of using biotinylated HLA-E*0103-YLLPAIVHI peptide instead of HLA-A2-peptide mix to eliminate HLA-E cross-reacting phage antibodies. In addition, unbiotinylated HLA-E*0103-YLLPAIVHI peptide was used for pre-blocking with beads containing the HLA-E*0103-VMAPRTLFL target.

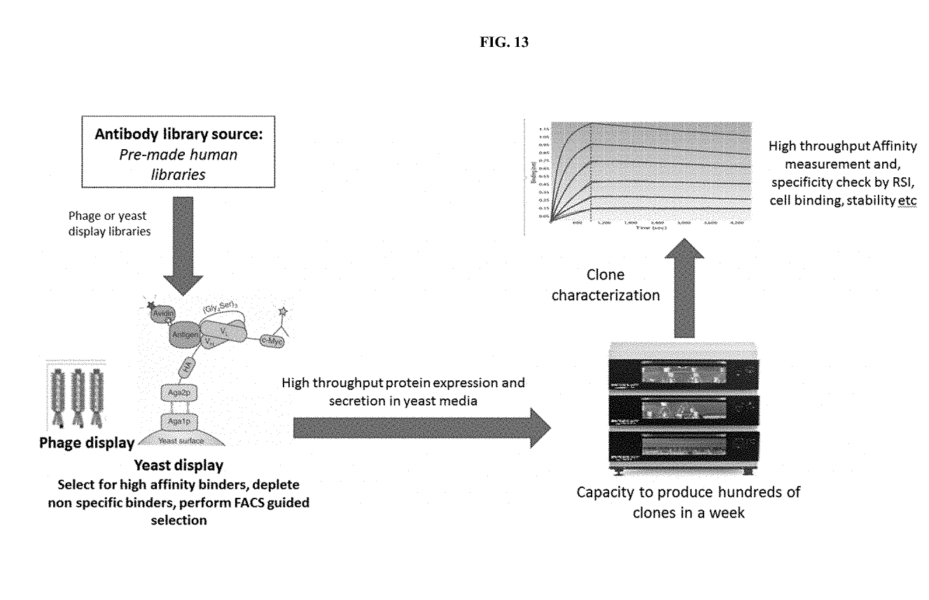

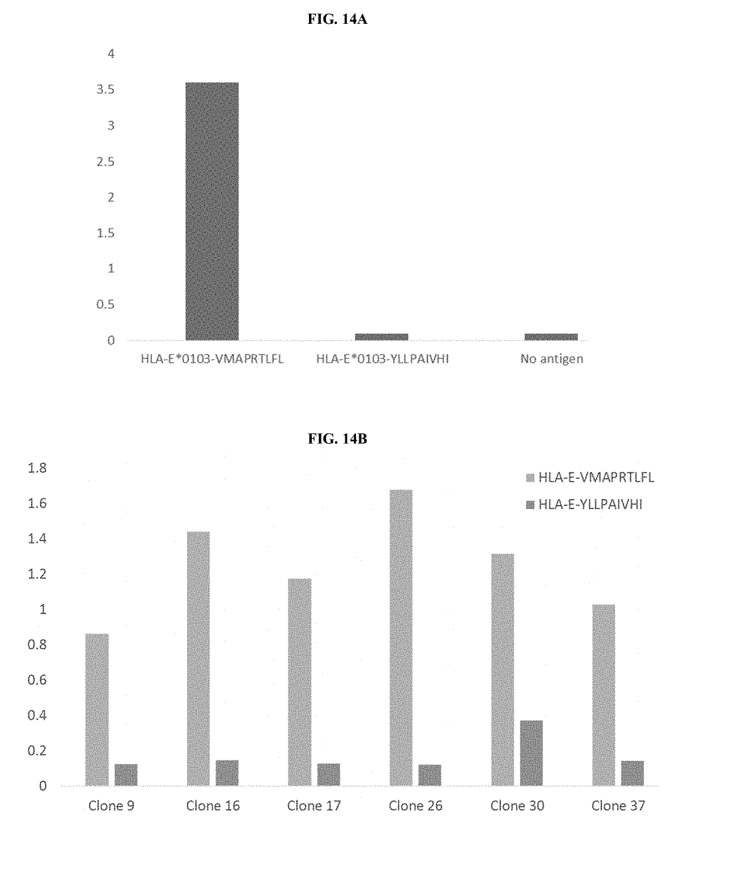

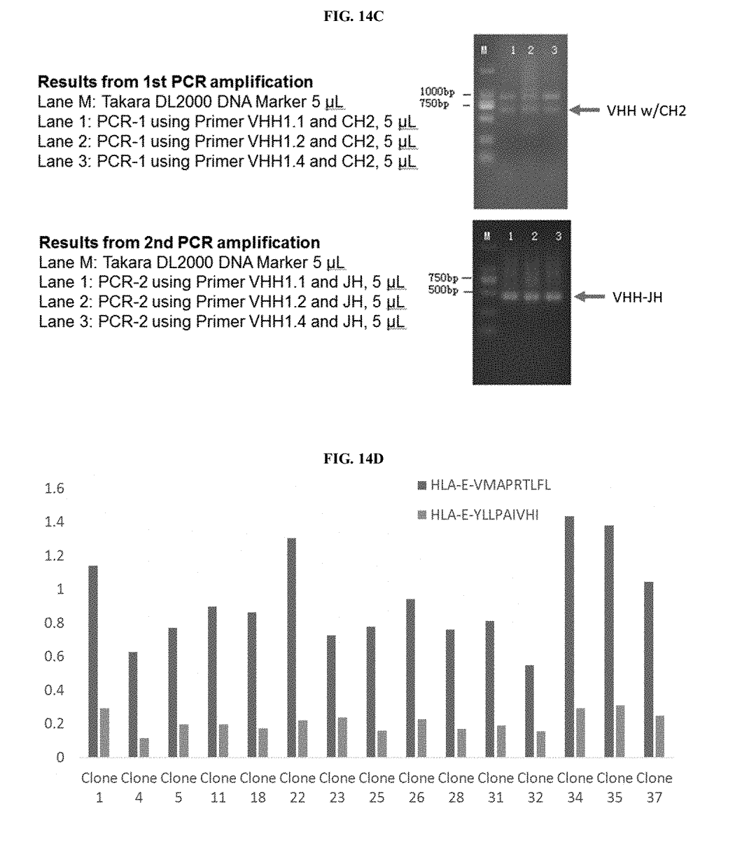

40 clones were picked from the output of the 4^{th} round of biopanning. A monoclonal phage ELISA was performed as shown in FIG. 14A. One unique clone determined by gene sequence was identified and subsequently expressed as a scFv in E. coli and as an IgG1 in HEK293 cells for downstream characterization. FIG. 14A-FIG. 14D illustrate human antibody scFv ELISA data for R4, mouse scFv library and VHH library to HLA-E-VMAPRTLFL

Discovery of Antibody Binders from an Immunized Mouse Phage Library:

The T-cell receptor-like antibodies were generated by first immunizing mice, followed by constructing an antibody library for display by bacteriophage. An effective amount of immunogen comprising a monomer of one peptide/HLA-E complex, wherein the peptide is the peptide of interest, was administered to a host for eliciting an immune response, wherein the immunogen retains a three-dimensional form thereof for a period of time sufficient to elicit an immune response against the three-dimensional presentation of the peptide in the binding groove of the HLA-E molecule. Serum collected from the host was then assayed to determine if desired antibodies that recognize a three-dimensional presentation of the peptide in the binding groove of the HLA-E molecule is being produced, wherein the desired antibodies differentiate the peptide/HLA-E complex from the HLA-E molecule alone, the peptide of interest alone, and a complex of HLA-E and irrelevant peptide. The mouse spleen was then isolated from immunized animal and an antibody library was constructed using bacteriophage or yeast or other display systems.

In general, four female Balb/c mice (also use mouse strains such as Bk/6, CD-1, and CFW) were immunized and the spleen from the best responder was selected for library construction. Briefly, mice were immunized 3x subcutaneously at 3-week intervals receiving 50 μg/injection of antigen HLA-E-VMAPRTLFL in monomer form. One week after the final injection, serum from immunized mice was collected and tested for antibody response to HLA-E-VMAPRTLFL by ELISA. The titer reached in several of the immunized mice was greater than 1:102,000 and the spleen from the best responsive mouse was removed and used to construct the scFv antibody phage library. Total RNA was isolated using the TriZol method and RNA was then assessed for quality by gel electrophoresis.

PCR amplification of mouse VH and VL genes was performed next. In brief, VH and VL genes were amplified from cDNA template using murine specific primers. The scFv cassettes were assembled by over-lapping PCR. scFv genes and phagemid (pHEN1) were digested using restriction enzymes and ligated together with T4 DNA ligase. The ligation mix was desalted and re-suspended in distilled water before being used to electro-transform TG1 E. coli competent cells to construct final library. Finally, phage displaying scFv proteins were packaged with helper phage M13KO7 following standard methods.

The quality of the library was assessed by QC-PCR using standard protocols. The results from the PCR assessment revealed that 30 of 30 clones carried the scFv insertion and 21 clones submitted for sequencing had intact scFv genes unique sequence data. Furthermore, the end library was determined to have a diversity of 5.5×10⁸.

### Library Screening:

The scFv phage display library generated via immunization with target HLA-E-VMAPRTLFL was used to select for specific binders using the In-solution biopanning technique. Just prior to working with library, phage sample was precipitated, centrifuged for 10 min at 5,000 g and the pelleted sample was resuspended in 2%M-PBS. The library was then sequentially added to 1 ml—Dynabeads (MyOne Streptavidin T1) and after 1 hr incubation at room temperature the tube was placed in a magnetic rack for 1 minute to remove non-specific binding phage with beads. The aspirated supernatant containing phage was then added to beads containing blocking buffer, 2% M-PBS and step from #1 (1 ml—Dynabeads (MyOne Streptavidin T1)) was repeated. Finally, the supernatant containing phage was mixed with streptavidin coated beads and incubated with 4 biotinylated targets (all HLA-A2-peptide complexes) to deplete non-specifically binding phage. Included in step 3 was unbiotinylated HLA-A2-peptide that was used as a blocking reagent.

In the first two rounds of biopanning, depletion was carried out using biotin-labeled HLA-A2-peptide in addition to inclusion of a blocking strategy using a non-biotin-labeled HLA-A2-peptide mix to remove and prevent binding of non-specific scFv expressing phage in the library. Following this step, positive panning for the target using biotin-labeled HLA-E-VMAPRTLFL was performed. In parallel, panning of same immune scFv phage library against groups with no coating (no antigen) and coating with biotin-labeled HLA-A2-peptide was performed. Some enrichment was observed with differences between the target group and negative control screening groups. The third round of biopanning was performed next using biotin-labeled HLA-E-YLLPAIVHI for depletion and pre-blocking. After that, positive panning for biotin-labeled HLA-E-VMAPRTLFL was performed. In parallel the library was again panned against two control groups: no coating and coating with biotin-labeled HLA-E-YLLPAIVHI. Enrichment was observed between the target group and control screening groups. 40 clones were selected from the third round of elution output to validate the specificity of enrichment, 13 clones bound to the positive target HLA-E-VMAPRTLFL and 6 clones from that group had unique sequence (FIG. 14B).

Antibody binders were also discovered from a different immunized mouse scFv library. Four female Balb/c mice were immunized 3× subcutaneously at 3-week intervals receiving 50 μg/injection of antigen HLA-E-ILSPTVVSI in monomer form. One week after the final injection, sera from immunized mice was collected and tested for antibody response by ELISA. The titer reached in several of the immunized mice was greater than 1:102,000 and the spleen from the best responsive mouse was removed and used to construct the scFv antibody phage library. Total RNA was isolated using the TriZol method and RNA was then assessed by gel electrophoresis.

PCR amplification was performed next. In brief, VH and VL genes were amplified from cDNA template using murine specific primers. The scFv cassettes were assembled by over-lapping PCR. scFv genes and phagemid (pHEN1) were digested using restriction enzymes and ligated together with T4 DNA ligase. The ligation mix was desalted and re-suspended in distilled water before being used to electro-transform TG1 E. coli competent cells to construct final library. Finally, phage displaying scFv proteins were packaged with the aid of helper phage M13Ko7 following standard methods.

The quality of the library was assessed by QC-PCR. The results from this assessment revealed that 30 of 30 clones carried the scFv insertion and 21 clones submitted for sequencing had intact scFv genes and were all unique. Furthermore, the end library was determined to have a diversity of 5.5×10⁸.

### Library Screening:

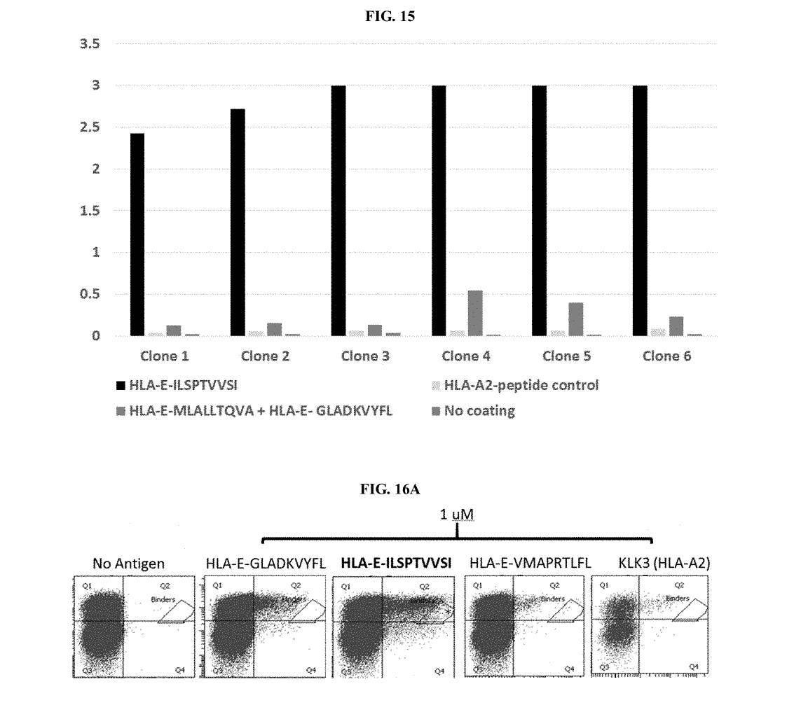

The scFv phage display library generated via immunization with target HLA-E-ILSPTVVSI was used to select for specific binders. In the first two rounds of biopanning, depletion was carried out using biotin-labeled HLA-A2-MLCKMGFAV peptide in addition to inclusion of a blocking strategy using non-biotin-labeled HLA-A2-MLCKMGFAV peptide to remove and prevent binding of non-specific scFv expressing phage in the library. Following this step, positive panning for the target biotin-labeled HLA-E-ILSPTVVSI was performed. In parallel, panning of same scFv phage library against groups with no coating (no antigen) and coating with biotin-labeled HLA-A2-MLCKMGFAV peptide was performed. Obvious enrichment was observed with clear differences between the target group and control screening groups (Input: 8×10¹¹; Output: HLA-E-ILSPTVVSI=1.5×10⁷; HLA-A2-MLCKMGFAC=8.0×10⁵; No antigen—1.1×10⁶). The third round of biopanning was performed next using biotin-labeled HLA-E-MLALLTQVA and HLA-E-GLADKVYFL for depletion and pre-blocking. After that, positive panning for biotin-labeled HLA-E-ILSPTVVSI was performed. In parallel the library was again panned against two control groups; no coating and coating with biotin-labeled HLA-E- MLALLTQVA and HLA-E-GLADKVYFL. Obvious enrichment was observed with good differences between the target group and control screening groups (Input—7.0×10¹¹; Output: HLA-E-ILSPTVVSI=3.8×10⁷; HLA-E-MLALLTQVA+HLA-E-GLADKVYFL=4.1×10⁶; No antigen=3.0×10⁶). 40 clones were selected from the third round of elution output to validate the specificity of enrichment, 21 clones bound to the positive target HLA-E-ILSPTVVSI and 6 clones from that group had unique sequence (FIG. 15).

### Immunized Llama Antibody Library:

Single domain antibodies from an immunized llamas phage library were also generated. A llama was immunized with HLA-E-VMAPRTLFL tetramers and both phage and yeast display libraries were constructed. FIG. 14D illustrates discovery of binders to the HLA-E-VMAPRTLFL target.

An effective amount of immunogen was also formed using peptide/HLA-E tetramers formed using biotinylated monomers with avidin or derivatives of avidin such as streptavidin and neutravidin to form tetramer complexes of peptide/HLA-E. The immunogen was prepared with Magic^{TM} adjuvant (Creative BioLabs) and subcutaneously administered to animals for eliciting an immune response, wherein the immunogen retains a three-dimensional form of the peptide/HLA-E complex thereof for a period of time sufficient to elicit an immune response against the three-dimensional presentation of the peptide in the binding groove of the HLA-E MHC I molecule. In the case of generating VHH single domain antibodies, a llama was immunized weekly for 6-weeks with 200 μg of HLA-E-VMAPRTLFL tetramer (refolded biotin-labeled monomer added to streptavidin at a ratio of 6:1) and prior to immunization, the HLA-E-peptide material was diluted 1:1 with Magic Adjuvant.

Pre-immunization and post-immunization serum was collected to monitor the antibody response by ELISA. The titer to HLA-E-VMAPRTLFL target was >100,000. 200 ml of blood was removed and total RNA isolated using the TriZol method. The total RNA was evaluated by gel electrophoresis and shown to be of high quality. The VHH genes were amplified by two rounds of PCR after reverse transcription using the unique forward primers:

VHH1.1:
(SEQ ID NO: 50)
5′CATGCCATGACTCGCGGCCCAGCCGGCCATGGCCCAGGTGCAGCTGGT
GCAGTCTGG-3′(sfil);
VHH1.2:
(SEQ ID NO: 51)
5′CATGCCATGACTCGCGGCCCAGCCGGCCATGGCCCAGGTCACCTTGAA
GGAGTCTGG-3′(sfil);
VHH1.4:
(SEQ ID NO: 52)
5′-CATGCCATGACTGCCGGCCCAGCCGGCCATGGCCCAGGTGCAGCTGC
AGGAGTCGGG-3′(sfil) and the back primer CH2:

5′-CGCCATCAAGGTACCAGTTGA-3′ (SEQ ID NO: 53) for the first PCR reaction. For the second round of amplification, the same forward primers were used with the back primer JH: 5′-CCACGATTCTGCGGCCGCTGAGGAGAC(AG)GTGACCTGGGTCC-3′ (SEQ ID NO: 54) (not I).

The PCR results are shown in FIG. 14C. The PCR products and the phagemid DNA (pHEN1) were cut with restriction enzymes separately before being ligated together with T4 DNA ligase. The ligation mix was desalted and subjected to electro-transformation with E. coli TG1 as the host. M13K07 helper phage were used to package and display VHH proteins. The library was determined to have a diversity of 1.2×10⁸ and 48 clones were picked and PCR analysis was performed to detect the insertion rate of the target gene. It was determined that 47/48 clones had VHH gene inserts. The clones from the end library were subjected to DNA sequencing and aligned for full analysis of sequence. All clones were found to present unique sequences, indicating the construction of a high diversity library.

### Library Screening:

The VHH library was used to select for binders to the immunogen HLA-E-VMAPRTLFL. For the first two rounds of biopanning, depletion was carried out using biotin-labeled HLA-A2-MLCKMGFAV peptide in addition to inclusion of a blocking step using non-biotin-labeled HLA-A2-MLCKMGFAV peptide to remove and prevent binding of non-specific VHH expressing phage in the library. Following this step, positive panning for the target biotin-labeled HLA-E-VMAPRTLFL was performed. In parallel, panning of same phage library against groups with no coating (no antigen) and coating with biotin-labeled HLA-A2- MLCKMGFAV peptide were performed. Obvious enrichment was observed with clear differences between the target group and control screening groups (Input: 6×10¹¹; Output: HLA-E-VMAPRTLFLF=3.6×10⁷; HLA-A2-MLCKMGFAC=8.5×10⁶; No antigen—8.0×10⁶). The third round of biopanning was performed next using biotin-labeled HLA-E-YLLPAIVHI for depletion and pre-blocking. After that, positive panning for biotin-labeled HLA-E-VMAPRTLFL was performed. In parallel the library was again panned against two control groups: no coating and coating with biotin-labeled HLA-E-YLLPAIVHI. Enrichment was observed with small differences between the target group and control screening groups (Input—8×10¹¹; Output: HLA-E-VMAPRTLFLF=6.9×10⁸; HLA-A2-MLCKMGFAC=4.35×10⁸; No antigen=2.35×10⁸). 40 clones were selected from the third round of elution output to validate the specificity of enrichment, 23 clones bound to the positive target HLA-E-VMAPRTLFL and 15 clones from that group had unique sequence (FIG. 14D).

Unique clones identified were then screened using ResoSens label-free technology rank-order specific binders based on binding kinetics and binding specificity.

### Yeast Antibody Library Construction and General Selection Scheme of HLA-E-Peptide Complexes:

Mice and llamas were immunized with monomer of HLA-E-ILSPTVVSI peptide complex and tetramerized HLA-E-VMAPRTLFL peptide complex, respectively. scFv or single domain VHH antibodies were constructed by reverse transcription and PCR amplification of V genes. Antibody libraries were displayed as scFv or single domain VHH antibodies on the surface of yeast. Both mouse and llama libraries had antibodies displayed with c-terminus FLAG tag. Following standard procedures, antibody library size was determined.

The size of the transformed immune yeast library was ˜5×10⁸ and 3.5×10⁸ for the mouse and llama libraries, respectively.

Early rounds of selections were carried out with streptavidin coated MACS beads (Miltenyi biotech) while later round of selections were carried out on a FACS sorter.

Round 1 selection: Briefly, 2 ml of 500 nM of biotinylated HLA-E-ILSPTVVSI or HLA-E-VMAPRTLFL peptide complex was incubated with 10¹⁰ yeast cells (>10× of the realized library size) from mouse or llama library at room temperature for 20 minutes. Cells were spun down on a centrifuge and washed twice with 45 ml of PBS +0.1% BSA (PBSB). Yeast cells were resuspended in 40 ml PBS +0.1% BSA (PBSB) and to suspended cells 0.5 ml of MACS beads was added followed by incubation at 4 C. for 15 minutes. Two washes with PBSB were performed before the cells were passed through a MACS column. Yeast bound to the column were eluted by removing the MACS column from the magnetic field and forcing the yeast cells out of the column with the aid of a plunger. Cells were then allowed to grow overnight in 50 ml selective media.

Round 2 selection: To eliminate non-specific binders to streptavidin and general framework of HLA-peptide complex, 10⁹ cells from the 1^{St} round of selection from the mouse and llama immunized libraries were incubated with 1 uM of biotinylated HLA-A2-peptide complex (negative control) and following washing and incubation with MACS beads, cells were subsequently passed through a MACS column. The flow-through yeast cells were collected and incubated with 250 nM of biotinylated HLA-E-ILSPTVVSI or with HLA-E-VMAPRTLFL peptide complex (target complexes) from immunized mouse and llama libraries, respectively at room temperature for 20 minutes. As in 1^{st} round of selection similar steps of washing, incubations with MACS beads, elution from MACS column were performed. Cells from both libraries were allowed to grow overnight in 50 ml selective media.

Round 3 selection: 5×10⁷ Yeast cells from round 2 mouse and llama libraries were incubated with 100 nM biotinylated HLA-E-ILSPTVVSI (mouse library) or HLA-E-VMAPRTLFL (llama library) for 20 minutes. Cells were washed and incubated with EA-PE (extravidin phycoerythrin) or SA-633 (Streptavidin alexa-633) for detection of biotinylated antigen and anti-FLAG-FITC for monitoring expression of scFv or VHH single domain antibodies. A separate yeast sample without any antigen but with the secondary reagents was used as a negative control. Appropriate sorting gate were drawn to collect the binders.

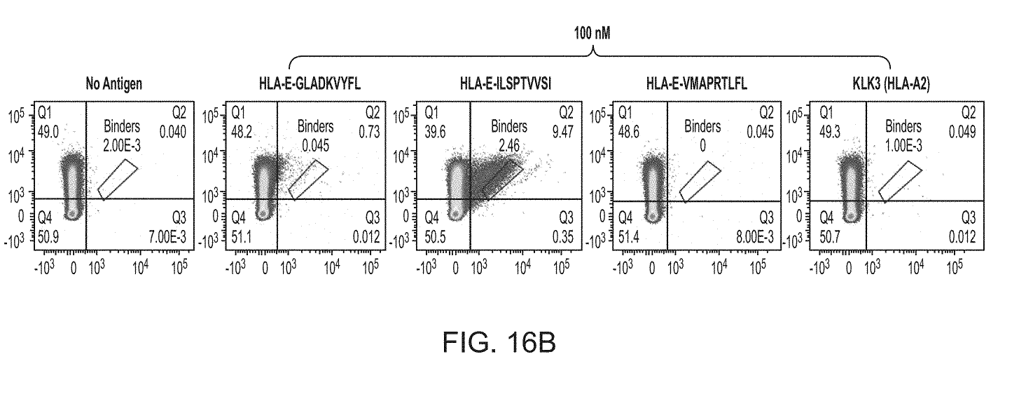

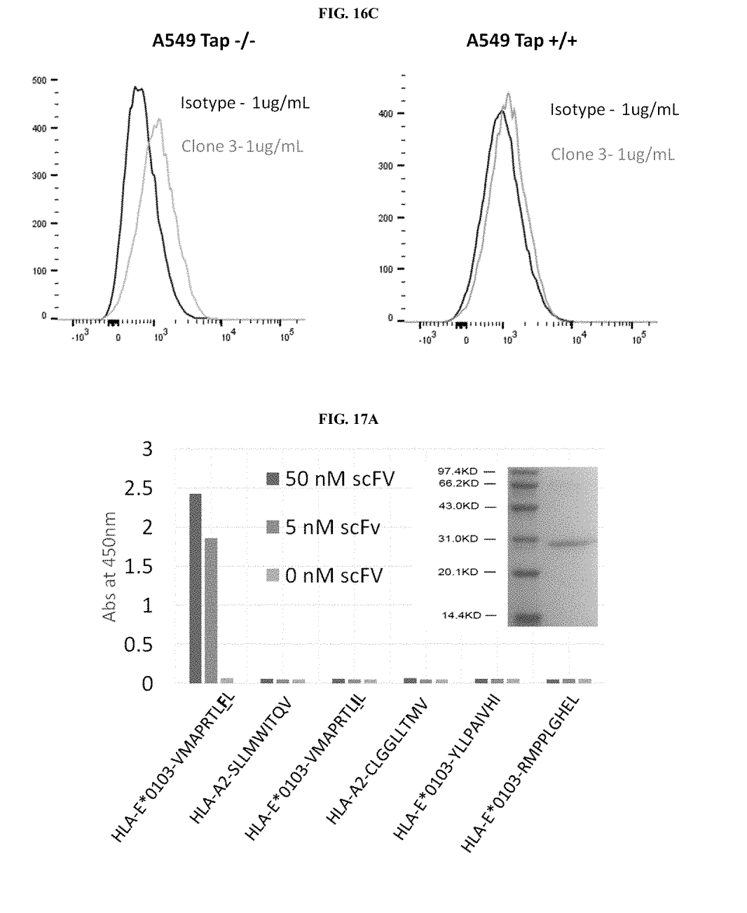

Round 4 selection: Samples containing 5×10⁷ yeast cells from round 3 from both mouse and llama libraries were incubated with 100 nM of either biotinylated HLA-E-ILSPTVVSI (mouse library) or HLA-E-VMAPRTLFL (llama library) peptide complexes for 20 minutes. Cells were washed and incubated with EA-PE or SA-633 for detection of biotinylated antigen and anti-FLAG-FITC for monitoring expression of scFv or VHH single domain antibodies. In addition to the negative control sample as mentioned in round 3, another yeast sample were incubated with negative controls, 100 nM biotinylated HLA-A2-peptide as well as with other HLA-E-peptide (e.g. for mouse library used HLA-E-VMAPRTLFL peptide complexes as a negative control and for llama library used HLA-E-ILSPTVVSI peptide complex as a negative control) . FACS sorting gate was drawn in a way that only events from the positive sample appear inside the gate ensuring specific target binders. FIG. 16A-FIG. 16B illustrate yeast from mouse immune library displaying scFv binders having binding specificity for the target, HLA-E-ILSPTVVSI peptide complex, after four rounds of selection.

Three clones were selected for expression as full length hIgG1 antibodies. The heavy and light chains from Clone 3 (anti-HLA-E-ILSPTVVSI complex) were cloned into pCDNA3.2 vectors (Thermo Fisher Scientific) and plasmids containing heavy and light chain genes were co-transfected into Expi293 (Thermo Fisher Scientific) to transiently express soluble Clone 3 hIgG1 antibody for purification on a

Protein A column. The purified sample preparation was assessed by SDS gel-electrophoresis under reducing conditions. After completion, the gel was stained with coomassie blue and revealed single heavy (˜50 kD) and light (25 kD) chains. Next purified Clone 3 was used to stain A549 lung cancer cells. In brief, cells were treated with 0.1 nM IFN for 48 hours before being harvested. Cells were counted and resuspended at 1×10⁷ cells/mL, 100 uL of cells were incubated at 4 C. for 1 hr with lug/ml of Clone 3 -hIgG1. Cells were washed 3× in PBS/10% FBS followed by a 30 min incubation with a 1:1000 dilution of anti-human Fc conjugated to APC before being washed three times and analyzed by flow cytometry (LSR). The binding specificity for Clone 3 antibody was assessed by label-free technology and shown to bind the specific target, HLA-E-ILSPTVVSI. In this example, the Clone 3 stains weakly to A549 lung cancer cells (KIFII antigen positive); however Clone 3 binding is significantly stronger to A549 cells that have TAP1 gene knocked out via gene editing using CRISPR/CAS9 technique (FIG. 16C). The TAP K/O cells express HLA-E as shown by staining with 3D12 antibody (see example 18). However, TAP-deficient cells load alternatively processed peptides into the HLA-E binding groove indicating that in TAP defective cancer cells, peptides derived from alternative processing pathways bind to HLA-E and are displayed on the cell surface for targeting.

### Example 18

### Characterization of TCR-Like Antibody to HLA-E-VMAPRTLFL Complex.

Human antibody, R4, isolated from the pre-made human library (see Example 17), was produced as an scFv and full-length IgG1, and purified and characterized for binding specificity, affinity and cell staining

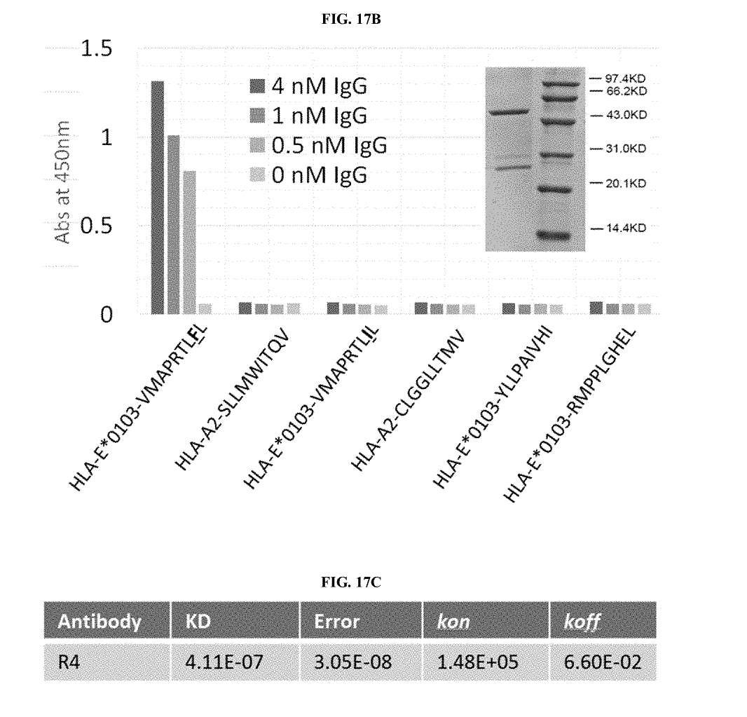

R4 human antibody expression, purification and binding specificity results are illustrated in FIG. 17A-FIG. 17C. The R4 scFv-6-his-tag (SEQ ID NO: 12) construct was cloned into pET25B plasmid, electro-transformed into Lemo21(DE3) Competent E. coli (New England BioLabs) for periplasmic expression and purification of soluble scFv on a NiNTA column (FIG. 17A). Heavy and light chains from R4 were cloned into pCDNA3.2 vectors (Thermo Fisher Scientific) and plasmids containing heavy and light chain genes were co-transfected into Expi293 (Thermo Fisher Scientific) to transiently express soluble R4 IgG1 antibody for purification on a Protein A column. Both purified sample preparations were assessed by SDS gel-electrophoresis under reducing conditions. After completion, gels were stained with coomassie blue and reveal a single ˜30 KD band observed for scFv (FIG. 17A) and heavy (˜50 kD) and light (25 kD) chains (FIG. 17B). Furthermore, the binding specificity for both R4 antibody forms was assessed by ELISA and shows antibody binding specific for the target, HLA-E-VMAPRTLFL. In FIG. 17C the affinity of R4 human antibody was determined by using Octet label-free technology (ForteBio) following the manufactures standard protocol and using streptavidin coated probes. The affinity for R4 human antibody was 4.1×10⁻⁷ M with a k-off rate of 6.6×10⁻² (min⁻¹). The R4 antibody affinity was further optimized by introducing random mutations into the CDR3H region resulting in the identification of clone #2 having binding affinity of KD=8.3×10⁻⁹M and a k-off rate of 2.82×10⁻⁴ min⁻¹.

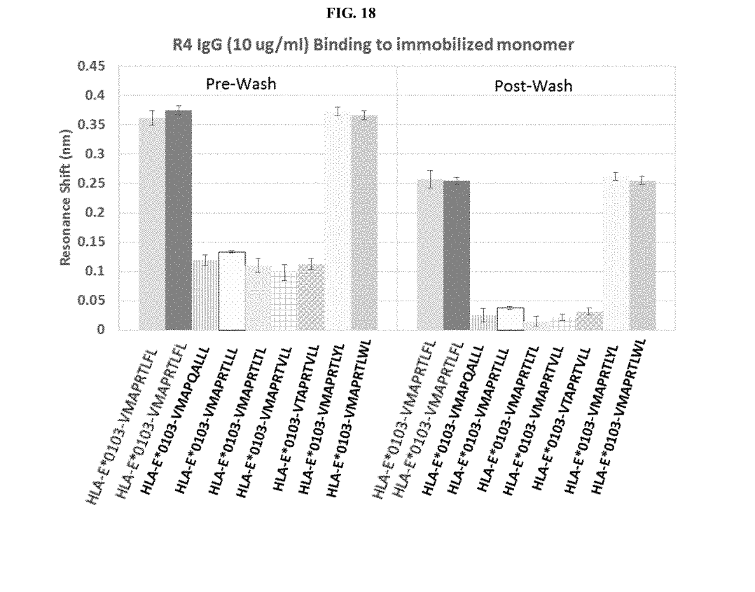

The next step was to characterize the fine binding specificity of R4 IgG1 antibody. Binding specificity was performed by immobilizing biotin-labeled HLA-E complexes loaded with similar peptide sequences to target peptide VMAPRTLFL (SEQ ID NO: 3). Similar peptides used had either a single amino acid substitution or more than one amino acid substitution. Moreover, the study was carried out to determine the R4 binding preference of amino acids in positions (p5) and (p8) in the peptide. Two control peptides, VMAPRTLYL (SEQ ID NO: 9) and VMAPRTLWL (SEQ ID NO: 10), were used to produce recombinant HLA-E complexes. These two peptides with highly conserved amino acid differences at position p8 do not exist in nature. They were selected for synthesis to assess the need for an aromatic ring containing peptide in position p8 of the peptide. The R4 antibody only showed binding to the specific peptide target, VMAPRTLFL (SEQ ID NO: 3) and the two closely related peptides VMAPRTYL (SEQ ID NO: 55) and VMAPRTLWL (SEQ ID NO: 10) while not binding to the other peptides. This indicates that R4 antibody binding to HLA-E-VMAPRTLFL peptide complex, in some instances, is dependent, on having an amino acid reside containing an aromatic ring structure in position p8 (FIG. 18)

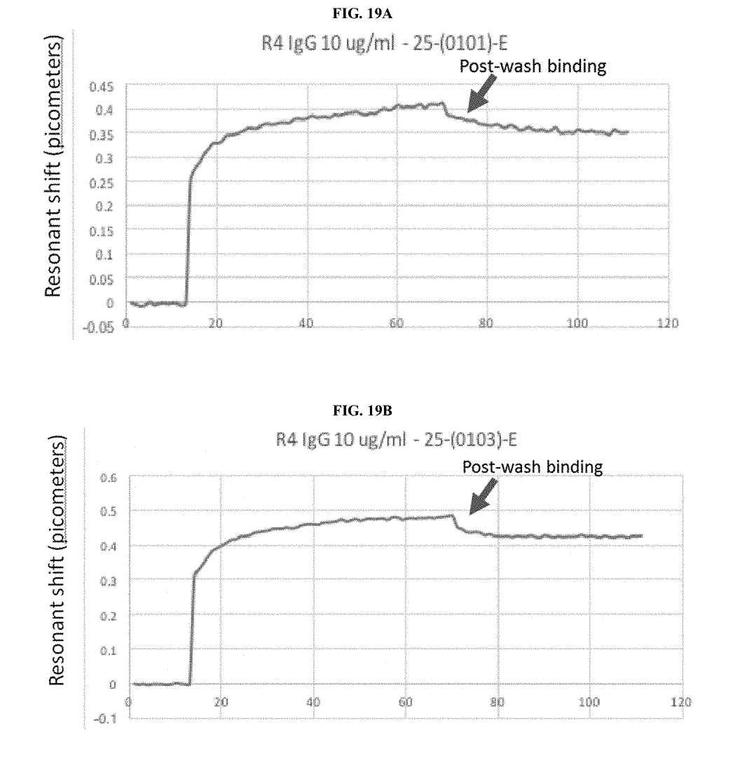

The results also exemplify that both HLA-E alleles (HLA-E*0101 and HLA-E*0103) are produced and loaded with the same peptide, VMAPRTLFL (SEQ ID NO: 3). Furthermore, FIG. 19A-FIG. 19B illustrates R4 antibody binding equivalency to HLA-E*0101 and HLA-E*0103 loaded with the VMAPRTLFL (SEQ ID NO: 3) peptide. In brief, biotin-labeled HLA-E*0101 and 0103-VMAPRTLFL complexes were immobilized on neutravidin coated bionetic plates. Then 10 ug/ml of R4 IgG1 antibody was added to well of bionetic plate and binding was observed in resonant shift units (pMeters) over 60 minutes. Wells were then washed with PBS and remaining bound R4 detected (post-wash binding). Antibodies that react to same peptide presented in both HLA-E*0101 and HLA-E*0103 alleles provide broad population coverage since nearly 100% of Homo sapiens express one or both of the HLA-E alleles.

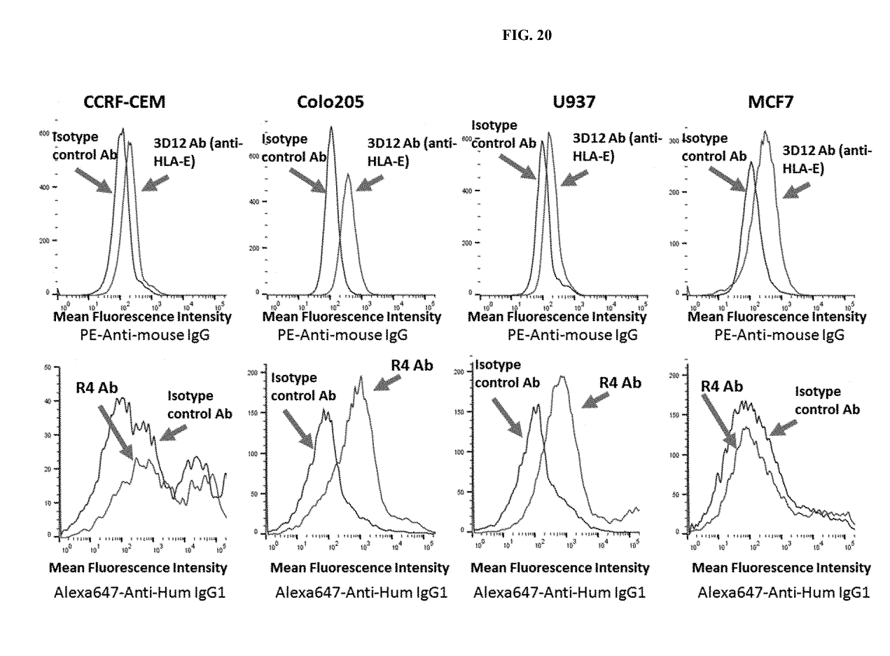

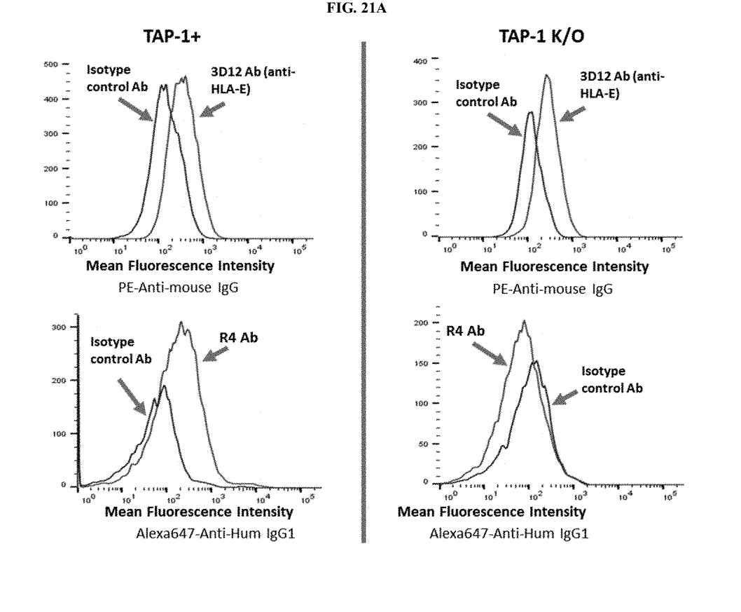

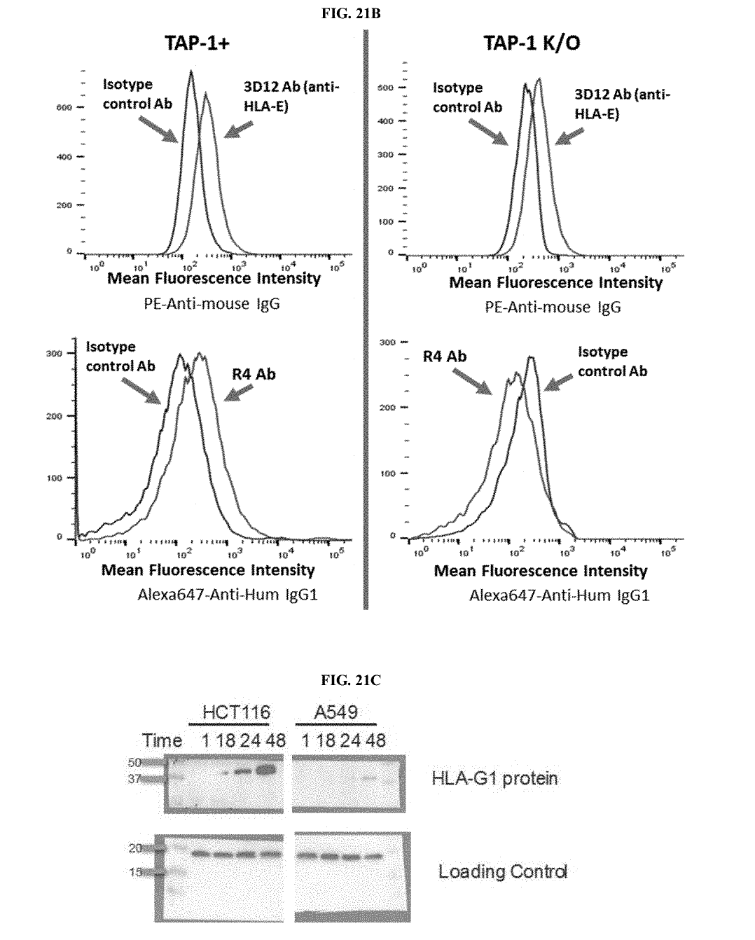

FIG. 20 illustrates R4 antibody being used to stain tumor cells. R4 IgG1 antibody binds to tumor cells that express HLA-E and the HLA-G protein, therefore the signal peptide from HLA-G is present and loaded into HLA-E. In contrast, tumor cells that do not express HLA-G are not stained with R4 antibody. Additionally, as illustrated in FIG. 21A-FIG. 21C, R4 antibody binds to HLA-E/G expressing HCT116 colorectal cancer cells and A549 lung cancer cells and does not bind to same cells that have TAP1 gene knocked out via gene editing using CRISPR/CAS9 technique. The TAP K/O cells express HLA-E as shown by staining with 3D12 antibody (top panels). However, defective TAP means the cells no longer transport the HLA-G signal peptide into the ER for loading into the HLA-E binding groove. This means that in TAP defective cancer cells, peptides derived from alternative processing pathways bind to HLA-E and displayed on the cell surface for targeting with the antibodies disclosed herein.

### Example 19

### Detection of Total HLA-E Protein Expression and HLA-E-Peptide Complex Specific Expression in Human Tumor Tissue.

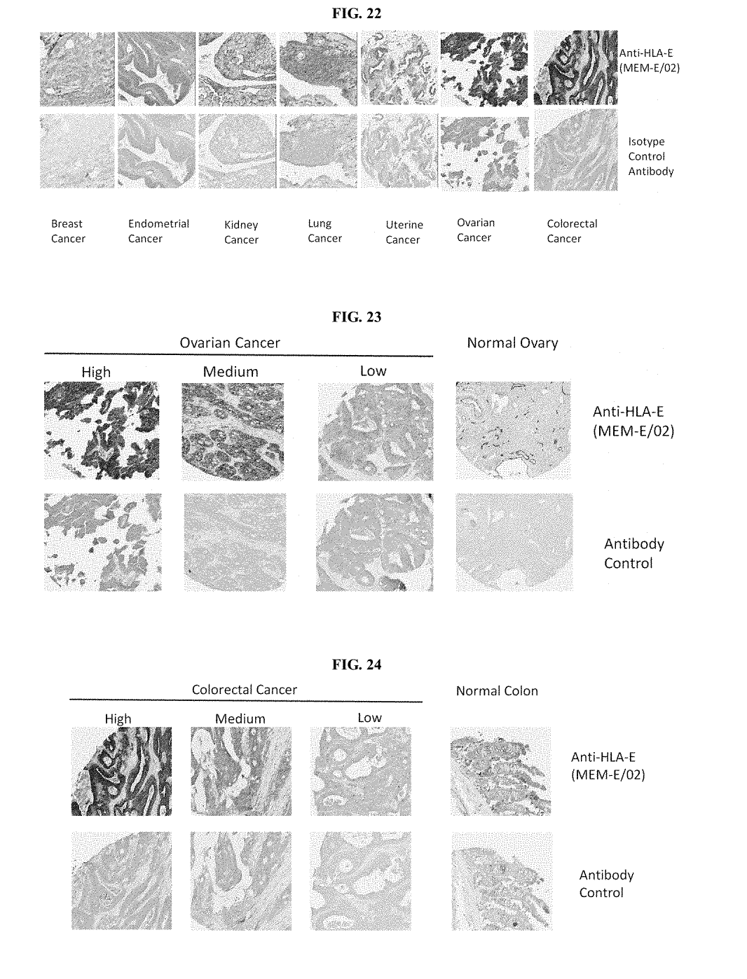

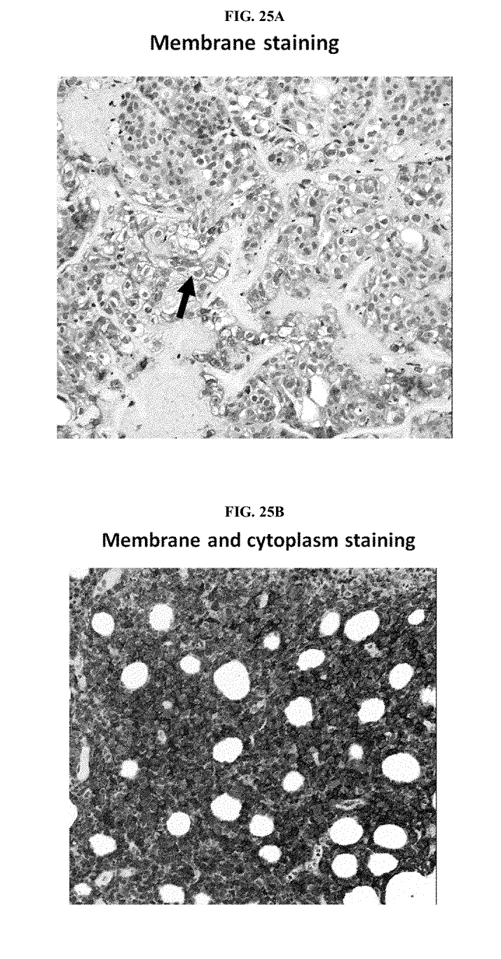

To determine total HLA-E protein expression (independent of peptide presented) immunocytochemistry staining was performed using monoclonal antibody MEM-E/02 (ThermoFisher Scientific) and formalin-fixed paraffin-embedded human tumor tissue microarrays (Origene Technologies and US BioMax). Detection of MEM-E/02 binding was determined using anti-mouse-HRP conjugated antibody (Abcam) and developed using 3,3′ Diaminobenzidine (DAB) substrate kit (Abcam). FIG. 22-FIG. 24 exemplify HLA-E expression in various cancers including lung, breast, ovarian and colorectal. Furthermore, in FIG. 25A-FIG. 25B, MEM-E/02 antibody stains HLA-E on membrane of breast cancer samples. Membrane expression of HLA-E-peptide targets is essential for developing TCR-like antibody-based drugs and targeting intracellular targets.

To evaluate TCR-like antibodies targeting HLA-E-peptide complexes, frozen human tumor tissue arrays are purchased from US BioMax and Origene. Frozen tissue sections made at 5-mm thickness are fixed using 5% methanol and stained with TCR-like antibody and control antibody at 1 μg/ml for 1 h in diluent containing 1.0% horse serum to prevent nonspecific staining of tissue. Detection of primary Ab binding is determined using goat anti-mouse Ig-HRP (ImmPRESS Anti-Mouse Ig-peroxidase Kit, Vector) that, in the presence of substrate chromagen (DAB), provides an indicator system (formation of brown precipitate) to visualize the location of Ag/Ab binding using light microscopy. Hematoxylin QS is used as a nuclear counterstain (Vector). H&E stains (Sigma-Aldrich, St. Louis, Mo.) are used to assess cell morphology and tumor cell presence in tissue. Tissue sections were analyzed using light microscopy (Nikon Eclipse TE 2000, inverted, deconvolution microscope with Simple PCI Suite software).

Scoring TCR-like antibody staining of human tissue to accurately reflect total cell staining and intensity, an in-house scoring protocol for TCR-like antibody has been implemented and followed. In this manner, a screening method consisting of proportion of stain (0-4) and intensity of stain (0-4) is established. A proportion of stain score of 0 represents no stain, a 1+ represents an average of 1-25 cells stained positive of 100 cells in the field (1-25%), a 2+ represents an average of 26-50 cells of 100 cells (26-50%), a 3+ score represents an average of 51-75 cells of 100 cells (51-75%), and a 4+ represents an average of 76-100 cells of 100 cells stained (76-100%). Intensity scores are based on a scale of 0-4, representing degrees of brown precipitate formed, in which 0 is negative, 1+ is weakly brown, 2+ is intermediately brown, 3+ is strongly brown, and 4+ is very dense brown. Finally, a total score (0-8) is determined by adding the scores for the proportion of stain and intensity of stain. Tissue sections are stained with TCR-like antibody and isotype controls at 1 μg/ml. The scores for proportion of stain and intensity of stain were reported as averages from five fields for each tissue sample.

### Example 20

### HLA-E-Peptide Complexes are Druggable Cancer Targets.

### Bispecific TCR-Like Antibodies:

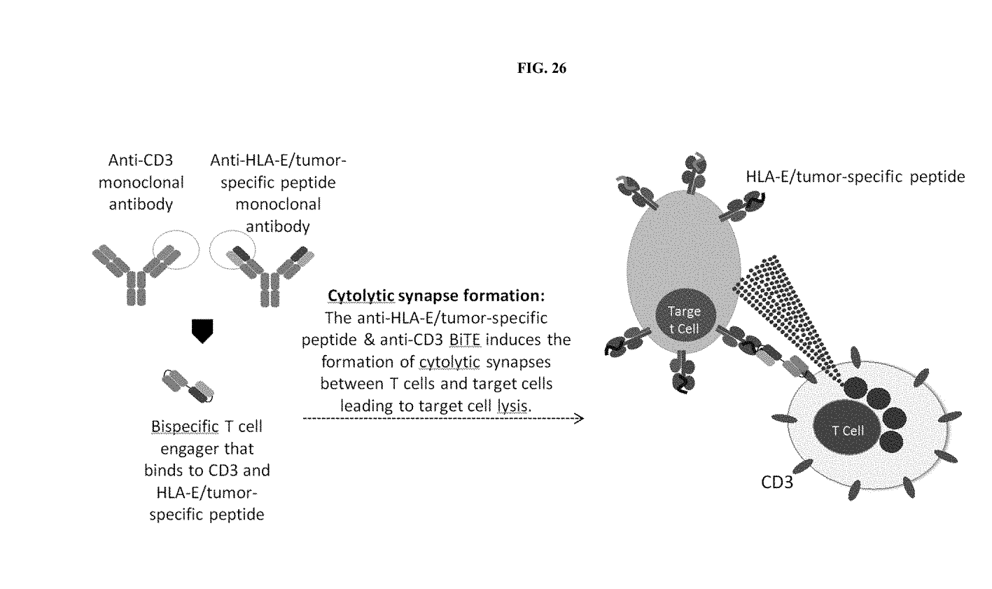

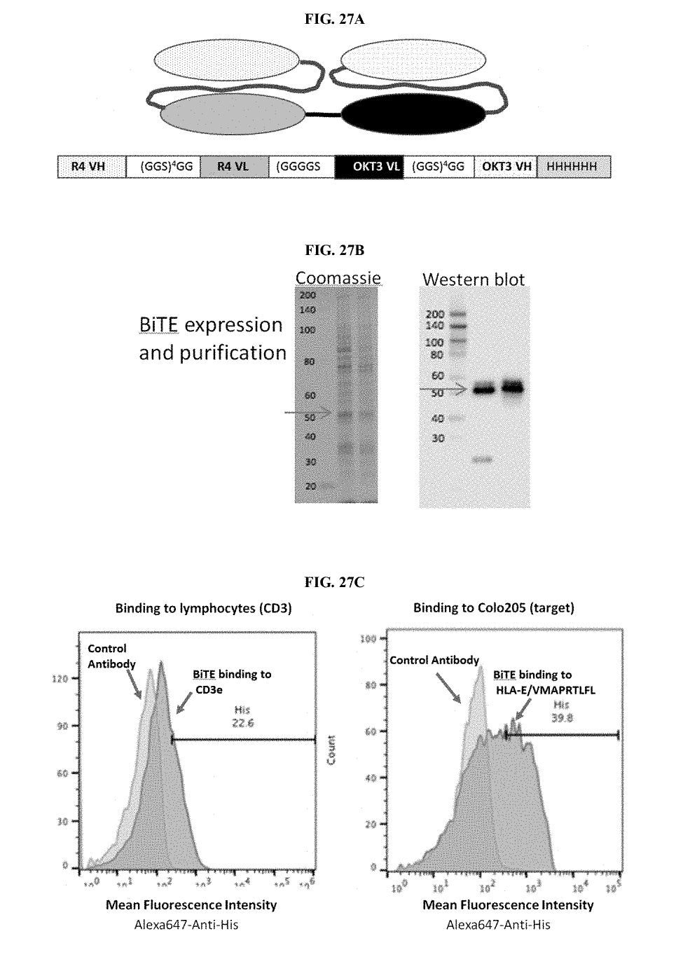

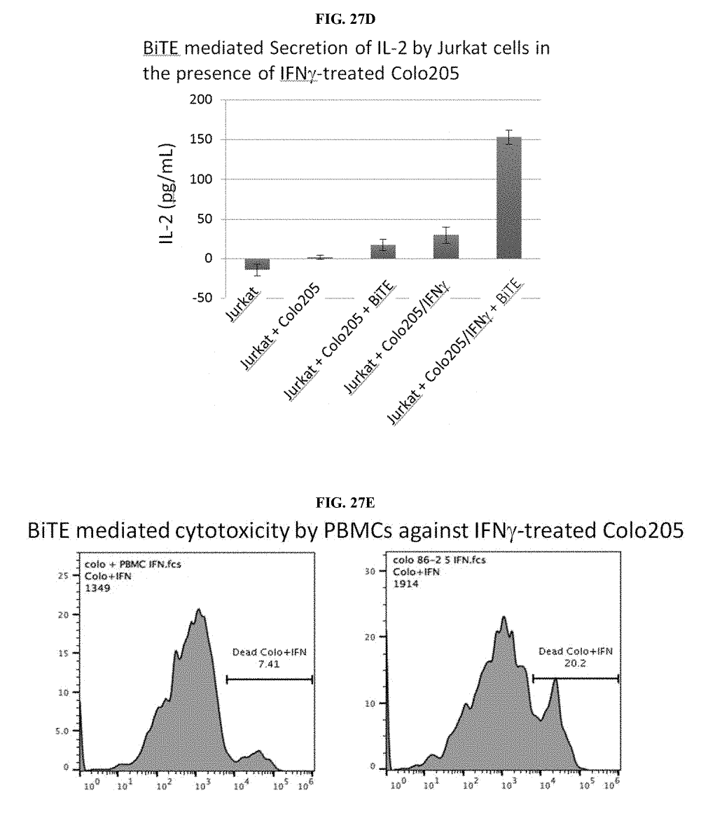

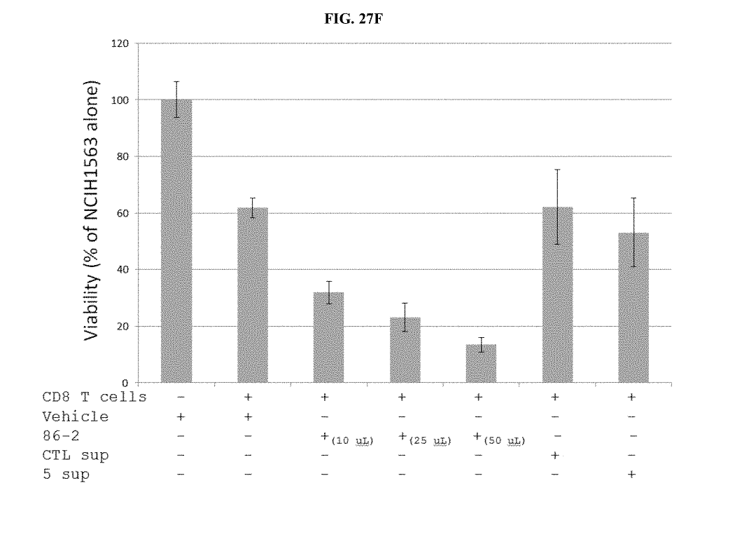

FIG. 26 illustrates an exemplary schematic for the generation of bispecific T-cell engager (BiTE) molecule. The clone, designated as BiTE 86-2 was constructed by recombinant DNA technology and purified from the supernatant from transfected 293 Expi cells. Purification of the BiTEs was performed using a cobalt resin chromatography column. BiTE 86-2 was validated by western blot using a horseradish peroxidase (HRP) conjugated anti-His antibody (Cell signaling technology), and by Coomassie staining (FIG. 27A-FIG. 27E). The specific binding of BiTEs to target cells was assessed by flow cytometry using an Alexa647-conjugated anti-His Tag antibody (Cell Signaling Technology). Binding to Jurkat cells, primary PBMCs, and Colo205 was assayed. Co-culture assays were performed in round bottom 96-well plates, containing 1×10⁴ target cells (Colo205 tumor cells). Purified BiTEs and 3×10⁴ Jurkats were added to the plates. After 14 hours, supernatant was collected for IL-2 release evaluation, which was performed by ELISA (IL-2 Human ELISA Kit, Thermo Fisher Scientific) following the manufacturer's instructions (FIG. 27D). For cytotoxic assays, PBMCs were stained with 0.05 μM Calcein AM in RPMI for 1 min at room temperature in a volume of 10 mL. Cells were then washed twice in complete medium and used in the flow cytometry-based cytotoxicity assays. Purified BiTEs and 15×10⁴ PBMCs were added to the plates. After fourteen hours, additional wells were used for the assessment of spontaneous apoptosis (target cells only and maximum target cell death (target cells only in 100 μL of complete medium plus 100 μL of 100% ethanol). 10 min before acquisition, 1 μL of 5 μM SYTOX red (Thermo Fisher Scientific) was added to each tube (FIG. 27A-FIG. 27E). For the co-culture flow cytometry assay, data were captured on an Attune NTX flow cytometer (Thermo Fisher Scientific) and analyzed using FlowJo software (Flowjo LLC).

In another study, BiTE 86-2 and related clone 5 (Sp34 (a-CD3e) VH-VL-R4 VL-VH) were used to kill NCIH-1563, lung cancer cells. For these assays, NCIH-1563 tumor cells were labeled with Calcein AM and incubated with 10×10⁴ human purified CD8+ cells (E:T=5). Purified BiTE 86-2 was added to wells at 10 ul (˜1.5 ug/ml), 25 ul (3.75 ug/ml, and 50 ul (7.5 ug/ml) and culture supernatant containing BiTE 5 was added to specific wells. All samples were tested in quadruplicate. The assay was incubated for 16 hr and tumor cell viability was determined by reading fluorescence at 485 nm excitation using a plate reader (FIG. 27F).

### Example 21

### Single-Domain Antibodies (VHH or Human Single-Domain Abs) Targeting HLA-E-Peptide Complexes And To Human CD3 Epsilon.

To generate single domain antibodies, llamas were immunized with monomer, tetramer or multimer formulations of HLA-E-peptide complexes. Subsequently, antibody libraries using phage and yeast display technology were constructed for selection of binders. Identified binders specific for the HLA-E-VMAPRTLFL peptide complex were expressed as VHH molecules in E. coli, and as VHH-Fc dimers in yeast and mammalian cells. Several unique (based on sequence data) VHH antibodies to HLA-E-VMAPRTLFL peptide target were discovered and the VHH genes cloned into pCDNA3.2 vector as a VHH-Fc construct and transfected Expi293 cells for production of dimeric molecules. The VHH-Fc antibody molecules were purified using a Protein-A affinity column. By linking the VHH to the hinge and Fc (lacking CH1) domains of the heavy chain and expressing as a dimer, the modified antibody is roughly half the size (75 kD) of a conventional mAb (150 kD). The lower molecular mass leads to better permeability in tissue without increasing renal clearance making these antibody molecules better at penetrating tumors. Additionally, their small size relative to conventional H and L chain antibodies make them highly conducive as multispecific and multivalent molecules.

VHH antibodies are expressed as VHH-Fc (bivalent) molecules and tested for binding specificity for HLA-E-peptide target. VHH molecules are also expressed as single domain antibodies containing a His-tag or as multispecific and multifunctional molecules. Bivalent dimers are made by the tandem fusion of two identical VHH antibodies. Combination of two VHH antibodies leads to construction of bivalent and bispecific molecules. Finally, because of the smaller size and therefore better tumor penetration ability, VHH and VHH-Fc molecules serve as desirable carriers for cytotoxic drugs (antibody drug conjugates).

Affinity and initial binding specificity for all VHH single domain antibodies are done using ELISAs and label-free assays. Finally, purified molecules are used to stain tumor tissues. In particular, anti-HLA-E-peptide antibody candidates are screened against patient tumor tissues for binding reactivity. Single domain T-cell-like antibodies that demonstrate a highly specific binding profile are used to develop multispecific molecules for treatment of cancer. These molecules are engineered as antibody-drug conjugates and as bispecific T cell engagers and assessed for anti-tumor activity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-39. (canceled)

40. A method of treating cancer in an individual in need thereof, comprising administering to the individual an antibody that selectively binds to a complex comprising a non-classical HLA-I and a neoantigen.

41. The method of claim 40, wherein the antibody does not have a binding affinity to (i) the non-classical HLA-I alone; or (ii) the neoantigen alone.

42-45. (canceled)

46. The method of claim 40, wherein the neoantigen comprises, consisting essentially of, or consisting of a sequence according to SEQ ID NO: 3 (VMAPRTLFL), SEQ ID NO: 13 (VMAPRTLIL), SEQ ID NO: 14 (VMPPRTLLL), SEQ ID NO: 31 (VMAPRTLVL), SEQ ID NO: 19 (YLLPRRGPRL), SEQ ID NO: 20 (AISPRTLNA), SEQ ID NO: 21 (SQAPLPCVL), SEQ ID NO: 15 (YLLEMLWRL), SEQ ID NO: 16 (YMLDLQPETT), SEQ ID NO: 22 (QMRPVSRVL), SEQ ID NO: 23 (ALALVRMLI), SEQ ID NO: 24 (SQQPYLQLQ), SEQ ID NO: 25 (AMAPIKTHL), SEQ ID NO: 26 (AMAPIKVRL), SEQ ID NO: 17 (YLLPAIVHI), SEQ ID NO: 27 (ILDQKINEV), SEQ ID NO: 28 (GVYDGEEHSV), SEQ ID NO: 29 (KVLEYVIKV), SEQ ID NO: 18 (SLLMWITQV), SEQ ID NO: 30 (YLEPGPVTV), SEQ ID NO: 32 (SLLEKSLGL), SEQ ID NO: 22 (QMRPVSRVL), SEQ ID NO: 33 (WIAAVTIAA), SEQ ID NO: 34 (TSDMPGTTL), SEQ ID NO: 35 (MLALLTQVA), SEQ ID NO: 36 (QMFEGPLAL), SEQ ID NO: 37 (VLWDRTFSL), SEQ ID NO: 38 (TLFFQQNAL), SEQ ID NO: 1 (GLADKVYFL), or SEQ ID NO: 2 (ILSPTVVSI).

47-53. (canceled)

54. The method of claim 40, wherein the non-classical HLA-I is HLA-E, HLA-F, HLA-G, or HLA-H.

55. The method of claim 40, wherein the non-classical HLA-I is HLA-E.

56. The method of claim 55, wherein the HLA-E is HLA-E*0101 or HLA-E*0103.

57. (canceled)

58. The method of claim 56, wherein the antibody selectively binds to the complex comprising:
(a) the HLA-E*0101 and the neoantigen;
(b) the HLA-E*0103 and the neoantigen; or
(c) the HLA-E*0101 and the neoantigen, and the HLA-E*0103 and the neoantigen.

59. The method of claim 55, wherein the complex comprises the HLA-E and VMAPRTLFL (SEQ ID NO: 3), HLA-E and VMAPRTLIL (SEQ ID NO: 13), HLA-E and VMPPRTLLL (SEQ ID NO: 14), HLA-E and VMAPRTLVL (SEQ ID NO: 31), HLA-E and YLLPRRGPRL (SEQ ID NO: 19), the HLA-E and AISPRTLNA (SEQ ID NO: 20), the HLA-E and SQAPLPCVL (SEQ ID NO: 21), the HLA-E and YLLEMLWRL (SEQ ID NO: 15), the HLA-E and YMLDLQPETT (SEQ ID NO: 16), the HLA-E and QMRPVSRVL (SEQ ID NO: 22), the HLA-E and ALALVRMLI (SEQ ID NO: 23), the HLA-E and SQQPYLQLQ (SEQ ID NO: 24), the HLA-E and AMAPIKTHL (SEQ ID NO: 25), the HLA-E and AMAPIKVRL (SEQ ID NO: 26), the HLA-E and YLLPAIVHI (SEQ ID NO: 17), the HLA-E and ILDQKINEV (SEQ ID NO: 27), the HLA-E and GVYDGEEHSV (SEQ ID NO: 28), the HLA-E and KVLEYVIKV (SEQ ID NO: 29), the HLA-E and SLLMWITQV (SEQ ID NO: 18), the HLA-E and YLEPGPVTV (SEQ ID NO: 30), the HLA-E and SLLEKSLGL (SEQ ID NO: 32), the HLA-E and QMRPVSRVL (SEQ ID NO: 22), the HLA-E and WIAAVTIAA (SEQ ID NO: 33), the HLA-E and TSDMPGTTL (SEQ ID NO: 34), the HLA-E and MLALLTQVA (SEQ ID NO: 35), the HLA-E and QMFEGPLAL (SEQ ID NO: 36), the HLA-E and VLWDRTFSL (SEQ ID NO: 37), the HLA-E and TLFFQQNAL (SEQ ID NO: 38), the HLA-E and GLADKVYFL (SEQ ID NO: 1), or the HLA-E and ILSPTVVSI (SEQ ID NO: 2).

60-66. (canceled)

67. The method of claim 40, wherein the antibody is a murine antibody, a chimeric antibody, a camelid antibody, a humanized antibody, or a human antibody.

68. The method of claim 40, wherein the antibody is a TCR-like antibody.

69. The method of claim 40, wherein the antibody is a single domain antibody.

70. The method of claim 69, wherein the single domain antibody is a camelid single domain antibody.

71. The method of claim 40, wherein the antibody is a multispecific antibody.

72. The method of claim 40, wherein the antibody is a multifunctional antibody.

73. The method of claim 40, wherein the antibody further comprises a conjugated therapeutic moiety.

74. The method of claim 40, wherein the selective binding of the antibody to the complex comprising the non-classical HLA-I and the neoantigen induces an immune response.

75. The method of claim 74, wherein the immune response comprises activation of T cells.

76-82. (canceled)

83. The method of claim 40, wherein the cancer is breast cancer, kidney cancer, lung cancer, ovarian cancer, colorectal cancer, or a B-cell malignancy.

84-173. (canceled)

174. The method of claim 40, wherein the antibody selectively binds to both the non-classical HLA-I and the neoantigen in the complex comprising the non-classical HLA-I and the neoantigen.


SEQUENCE LISTING (63 sequences)

SEQ ID NO: 1 - protein, 9 aa, Artificial Sequence
  Description of Artificial Sequence Synthetic peptide
GLADKVYFL

SEQ ID NO: 2 - protein, 9 aa, Artificial Sequence
  Description of Artificial Sequence Synthetic peptide
ILSPTVVSI

SEQ ID NO: 3 - protein, 9 aa, Artificial Sequence
  Description of Artificial Sequence Synthetic peptide
VMAPRTLFL

SEQ ID NO: 4 - protein, 9 aa, Artificial Sequence
  Description of Artificial Sequence Synthetic peptide
VMAPQALLL

SEQ ID NO: 5 - protein, 9 aa, Artificial Sequence
  Description of Artificial Sequence Synthetic peptide
VMAPRTLLL

SEQ ID NO: 6 - protein, 9 aa, Artificial Sequence
  Description of Artificial Sequence Synthetic peptide
VMAPRTLTL

SEQ ID NO: 7 - protein, 9 aa, Artificial Sequence
  Description of Artificial Sequence Synthetic peptide
VMAPRTVLL

SEQ ID NO: 8 - protein, 9 aa, Artificial Sequence
  Description of Artificial Sequence Synthetic peptide
VTAPRTVLL

SEQ ID NO: 9 - protein, 9 aa, Artificial Sequence
  Description of Artificial Sequence Synthetic peptide
VMAPRTLYL

SEQ ID NO: 10 - protein, 9 aa, Artificial Sequence
  Description of Artificial Sequence Synthetic peptide
VMAPRTLWL

SEQ ID NO: 11 - protein, 16 aa, Artificial Sequence
  Description of Artificial Sequence Synthetic peptide
GGGSGGGSGGGSGGGS

SEQ ID NO: 12 - protein, 6 aa, Artificial Sequence
  Description of Artificial Sequence Synthetic 6xHis tag
HHHHHH

SEQ ID NO: 13 - protein, 9 aa, Artificial Sequence
  Description of Artificial Sequence Synthetic peptide
VMAPRTLIL

SEQ ID NO: 14 - protein, 9 aa, Artificial Sequence
  Description of Artificial Sequence Synthetic peptide
VMPPRTLLL

SEQ ID NO: 15 - protein, 9 aa, Artificial Sequence
  Description of Artificial Sequence Synthetic peptide
YLLEMLWRL

SEQ ID NO: 16 - protein, 10 aa, Artificial Sequence
  Description of Artificial Sequence Synthetic peptide
YMLDLQPETT

SEQ ID NO: 17 - protein, 9 aa, Artificial Sequence
  Description of Artificial Sequence Synthetic peptide
YLLPAIVHI

SEQ ID NO: 18 - protein, 9 aa, Artificial Sequence
  Description of Artificial Sequence Synthetic peptide
SLLMWITQV

SEQ ID NO: 19 - protein, 10 aa, Artificial Sequence
  Description of Artificial Sequence Synthetic peptide
YLLPRRGPRL

SEQ ID NO: 20 - protein, 9 aa, Artificial Sequence
  Description of Artificial Sequence Synthetic peptide
AISPRTLNA

SEQ ID NO: 21 - protein, 9 aa, Artificial Sequence
  Description of Artificial Sequence Synthetic peptide
SQAPLPCVL

SEQ ID NO: 22 - protein, 9 aa, Artificial Sequence
  Description of Artificial Sequence Synthetic peptide
QMRPVSRVL

SEQ ID NO: 23 - protein, 9 aa, Artificial Sequence
  Description of Artificial Sequence Synthetic peptide
ALALVRMLI

SEQ ID NO: 24 - protein, 9 aa, Artificial Sequence
  Description of Artificial Sequence Synthetic peptide
SQQPYLQLQ

SEQ ID NO: 25 - protein, 9 aa, Artificial Sequence
  Description of Artificial Sequence Synthetic peptide
AMAPIKTHL

SEQ ID NO: 26 - protein, 9 aa, Artificial Sequence
  Description of Artificial Sequence Synthetic peptide
AMAPIKVRL

SEQ ID NO: 27 - protein, 9 aa, Artificial Sequence
  Description of Artificial Sequence Synthetic peptide
ILDQKINEV

SEQ ID NO: 28 - protein, 10 aa, Artificial Sequence
  Description of Artificial Sequence Synthetic peptide
GVYDGEEHSV

SEQ ID NO: 29 - protein, 9 aa, Artificial Sequence
  Description of Artificial Sequence Synthetic peptide
KVLEYVIKV

SEQ ID NO: 30 - protein, 9 aa, Artificial Sequence
  Description of Artificial Sequence Synthetic peptide
YLEPGPVTV

SEQ ID NO: 31 - protein, 9 aa, Artificial Sequence
  Description of Artificial Sequence Synthetic peptide
VMAPRTLVL

SEQ ID NO: 32 - protein, 9 aa, Artificial Sequence
  Description of Artificial Sequence Synthetic peptide
SLLEKSLGL

SEQ ID NO: 33 - protein, 9 aa, Artificial Sequence
  Description of Artificial Sequence Synthetic peptide
WIAAVTIAA

SEQ ID NO: 34 - protein, 9 aa, Artificial Sequence
  Description of Artificial Sequence Synthetic peptide
TSDMPGTTL

SEQ ID NO: 35 - protein, 9 aa, Artificial Sequence
  Description of Artificial Sequence Synthetic peptide
MLALLTQVA

SEQ ID NO: 36 - protein, 9 aa, Artificial Sequence
  Description of Artificial Sequence Synthetic peptide
QMFEGPLAL

SEQ ID NO: 37 - protein, 9 aa, Artificial Sequence
  Description of Artificial Sequence Synthetic peptide
VLWDRTFSL

SEQ ID NO: 38 - protein, 9 aa, Artificial Sequence
  Description of Artificial Sequence Synthetic peptide
TLFFQQNAL

SEQ ID NO: 39 - protein, 9 aa, Artificial Sequence
  Description of Artificial Sequence Synthetic peptide
VMAPCTLLL

SEQ ID NO: 40 - protein, 9 aa, Artificial Sequence
  Description of Artificial Sequence Synthetic peptide
RAHYNIVTF

SEQ ID NO: 41 - DNA, 21 bp, Artificial Sequence
  Description of Artificial Sequence Synthetic primer
gacattgtgatgacccagtct

SEQ ID NO: 42 - DNA, 21 bp, Artificial Sequence
  Description of Artificial Sequence Synthetic primer
ggatacagttggtgcagcatc

SEQ ID NO: 43 - DNA, 20 bp, Artificial Sequence
  Description of Artificial Sequence Synthetic primer
caggtgcagctgaagcagtc

SEQ ID NO: 44 - DNA, 39 bp, Artificial Sequence
  Description of Artificial Sequence Synthetic primer
ggccagtggatagtcagatgggggtgtcgtcgttttggc

SEQ ID NO: 45 - DNA, 33 bp, Artificial Sequence
  Description of Artificial Sequence Synthetic primer
agtcataagcttgacattgtgatgacccagtct

SEQ ID NO: 46 - DNA, 33 bp, Artificial Sequence
  Description of Artificial Sequence Synthetic primer
agtcatctcgagggccagtggatagtcagatgg

SEQ ID NO: 47 - protein, 9 aa, Artificial Sequence
  Description of Artificial Sequence Synthetic peptide
RMPPLGHEL

SEQ ID NO: 48 - protein, 9 aa, Artificial Sequence
  Description of Artificial Sequence Synthetic peptide
VMTTVLATL

SEQ ID NO: 49 - protein, 9 aa, Artificial Sequence
  Description of Artificial Sequence Synthetic peptide
RLPAKAPLL

SEQ ID NO: 50 - DNA, 57 bp, Artificial Sequence
  Description of Artificial Sequence Synthetic primer
catgccatgactcgcggcccagccggccatggcccaggtgcagctggtgcagtctgg

SEQ ID NO: 51 - DNA, 57 bp, Artificial Sequence
  Description of Artificial Sequence Synthetic primer
catgccatgactcgcggcccagccggccatggcccaggtcaccttgaaggagtctgg

SEQ ID NO: 52 - DNA, 57 bp, Artificial Sequence
  Description of Artificial Sequence Synthetic primer
catgccatgactgccggcccagccggccatggcccaggtgcagctgcaggagtcggg

SEQ ID NO: 53 - DNA, 21 bp, Artificial Sequence
  Description of Artificial Sequence Synthetic primer
cgccatcaaggtaccagttga

SEQ ID NO: 54 - DNA, 41 bp, Artificial Sequence
  Description of Artificial Sequence Synthetic primer
ccacgattctgcggccgctgaggagacrgtgacctgggtcc

SEQ ID NO: 55 - protein, 8 aa, Artificial Sequence
  Description of Artificial Sequence Synthetic peptide
VMAPRTYL

SEQ ID NO: 56 - protein, 28 aa, Artificial Sequence
  Description of Artificial Sequence Synthetic peptide
MAVMAPRTLVLLLSGALALTQTWAGSHS

SEQ ID NO: 57 - protein, 24 aa, Artificial Sequence
  Description of Artificial Sequence Synthetic peptide
MAVMAPRTLVLLLSGALALTQTWA

SEQ ID NO: 58 - protein, 14 aa, Artificial Sequence
  Description of Artificial Sequence Synthetic peptide
MAVMAPRTLVLLLS

SEQ ID NO: 59 - protein, 19 aa, Artificial Sequence
  Description of Artificial Sequence Synthetic peptide
EIINVGHSFHVNFEDNDNR

SEQ ID NO: 60 - protein, 15 aa, Artificial Sequence
  Description of Artificial Sequence Synthetic peptide
GGGGSGGGGSGGGGS

SEQ ID NO: 61 - protein, 14 aa, Artificial Sequence
  Description of Artificial Sequence Synthetic peptide
GGSGGSGGSGGSGG

SEQ ID NO: 62 - protein, 5 aa, Artificial Sequence
  Description of Artificial Sequence Synthetic peptide
GGGGS

SEQ ID NO: 63 - protein, 9 aa, Artificial Sequence
  Description of Artificial Sequence Synthetic peptide
KIFGSLAFL